(19) United States
(12) Patent Application Publication
(10) Pub. No.: US 2023/0404098 A1
(43) Pub. Date: Dec. 21, 2023

(54) DAIRY-LIKE COMPOSITIONS AND RELATED METHODS

(71) Applicant: New Culture Inc., San Leandro, CA (US)

(72) Inventors: Inja RADMAN, Oakland, CA (US); Neil ADAMES, San Leandro, CA (US); Patrick STODDARD, San Francisco, CA (US); Dilrajkaur PANFAIR, San Jose, CA (US)

(73) Assignee: New Culture Inc., San Leandro, CA (US)

(21) Appl. No.: 18/035,257

(22) PCT Filed: Nov. 4, 2021
(86) PCT No.: PCT/US2021/058004

§ 371 Date: May 3, 2023

(51) Int. Cl. A23C 20/00; C07K 14/47; A23J 3/10; A23J 3/28; A23J 3/22; A23J 3/34

(52) U.S. Cl. CPC A23C 20/00; C07K 14/4732; A23J 3/10; A23J 3/225; A23J 3/222; A23J 3/344; A23J 3/285

(57) ABSTRACT

Provided herein are dairy-like compositions and the methods of making the same using one or more recombinant proteins.

### CROSS-REFERENCE

This application claims the benefit of U.S. Provisional Application No. 63/109,851, filed on Nov. 4, 2020, which is incorporated herein by reference in its entirety.

### BACKGROUND OF THE INVENTION

The clean food space is comprised of both plant-based and cell-based foods. Cell-based food is a large umbrella term that includes culturing muscle and fat cells to replace slaughtered meat and culturing bioengineered organisms to express recombinant animal proteins to replace other animal products such as dairy and eggs. The need to find an alternate source of animal protein comes from the inefficiencies and unsustainability of current animal food production.

Global demand for dairy is continuing to increase all around the world. However, dairy farming impacts the environment negatively in several ways. Dairy cows and their manure produce greenhouse gases, particularly methane, which contribute to climate change. They also require unsustainable territories of land and gallons of water for their farming. Furthermore, poor handling of manure is known to pollute rivers and local fresh water, and farming practices themselves contribute to deforestation and degradation of wetlands and praries.

Cheese is the third most unsustainable animal product globally (when measuring greenhouse gas emissions per kg of product), and the consumption of dairy cheese has not been slowed down by plant-based alternatives introduced into the market in the last 10 years. On the contrary, mozzarella cheese consumption is growing year on year in the US and in developing markets. Current cheese alternatives do not match the functionality, nutrition and taste of dairy cheese due to their lack of casein proteins.

Similarly, the global market lacks functional alternatives for other dairy products such as yogurts. Plant-based alternatives have not been able to match the functionality, nutrition and taste of dairy products which leads consumers to continue using dairy products with a much larger environmental footprint.

### SUMMARY OF THE INVENTION

There is a need in the art for alternative cheese products that match the functionality, nutrition, and taste of dairy cheese. This disclosure meets this unmet need.

In some aspects, described herein may be consumable compositions made using hybrid micelles. In some aspects, described herein may be hybrid micelle compositions comprising at least two casein proteins. In some embodiments, the two casein proteins are from different mammalian species. The at least one of the casein proteins may be a recombinant protein. In some cases, the casein proteins may be associated in micellar form.

In some embodiments, the at least two casein proteins comprise a first alpha casein protein and a first kappa casein protein, wherein the first alpha casein protein and the first kappa casein protein may be from different mammalian species. In some embodiments, both of the first alpha casein protein and the first kappa casein protein may be recombinant proteins.

In some aspects, described herein are hybrid micelle compositions, comprising two or more recombinant casein proteins. In some embodiments, the at least two recombinant casein proteins may be from different mammalian species. In some embodiments, the composition comprises a first alpha casein protein and a first kappa casein protein. In some embodiments, the first alpha casein protein and the first kappa casein protein may be associated in micellar form.

In some embodiments, the first alpha casein protein and the first kappa casein protein may be from different mammalian species.

In some embodiments, the hybrid micelle comprises a second alpha casein protein. In some embodiments, the first alpha casein protein and the second alpha casein protein may be from different mammalian species. In some embodiments, the first alpha casein protein and the second alpha casein protein may be from the same mammalian species.

In some embodiments, the second alpha casein protein may be an alpha-S1-casein protein. In some embodiments, the second alpha casein protein may be an alpha-S2-casein protein. In some embodiments, the second alpha casein may be a truncated form of an alpha casein. In some embodiments, the second alpha casein comprises a N-terminal truncation of alpha-S1-casein protein. In some embodiments, the second alpha casein comprises lacks 1 to 59 N-terminal amino acids of alpha-S1-casein protein. In some embodiments, the second alpha-S1-casein protein comprises a bovine amino acid sequence starting at amino acid 23, 24, 25 or 26 of SEQ ID NO. 3. In some embodiments, the hybrid micelle further comprises a third alpha casein, wherein the third alpha casein comprises a N-terminal truncation of alpha-S1-casein protein. In some embodiments, the first alpha casein protein and the first kappa casein protein may be from different mammalian species.

In some embodiments, the hybrid micelle comprises a second kappa casein protein. In some embodiments, the first kappa casein protein and the second kappa casein protein may be from different mammalian species. In some embodiments, the first kappa casein protein and the second kappa casein protein may be from the same mammalian species.

In some embodiments, the first alpha casein protein may be an alpha-S1-casein protein. In some embodiments, the first alpha casein protein may be an alpha-S2-casein protein. In some embodiments, the first alpha casein protein may be a bovine alpha casein protein. In some embodiments, the first alpha casein protein comprises an amino acid sequence of SEQ ID NO. 1-11, 18-20, 30-32 or 39-41, or an amino acid sequence with at least 90% sequence identity to any one of SEQ ID NO. 1-11, 18-20, 30-32 or 39-41.

In some embodiments, the first kappa casein protein may be a kappa casein protein from any one of: ovine, caprine, equine, and camel kappa casein proteins. In some embodiments, the first kappa casein protein comprises an amino acid sequence of any one of SEQ ID NOs. 53-70, or an amino acid sequence with at least 90% sequence identity to any one of SEQ ID NOs. 53-70.

In some embodiments, the first alpha casein protein may be an ovine alpha casein protein. In some embodiments, the first alpha casein protein comprises an amino acid sequence of any one of SEQ ID NOs. 12-14 or 33-35 or an amino acid sequence with 90% sequence identity to any one of SEQ ID NOs. 12-14 or 33-35.

In some embodiments, the first kappa casein protein may be a kappa casein protein selected from the group consisting of bovine, caprine, equine, and camel. In some embodiments, the first kappa casein protein comprises an amino acid sequence according to any one of SEQ ID NOs. 48-52 or 56-70, or an amino acid sequence with at least 90% sequence identity to any one of SEQ ID NOs. 48-52 or 56-70.

In some embodiments, the first alpha casein protein may be a caprine alpha casein protein. In some embodiments, the first alpha casein protein comprises an amino acid sequence of any one of SEQ ID NOs. 15-17 or 36-38, or an amino acid sequence with at least 90% sequence identity to any one of SEQ ID NOs. 15-17 or 36-38.

In some embodiments, the first kappa casein protein may be a kappa casein protein selected from the group consisting of ovine, bovine, equine and camel. In some embodiments, the first kappa casein protein comprises an amino acid sequence of any one of SEQ ID NOs. 48-55 or 59-70 or an amino acid sequence with at least 90% sequence identity to any one of SEQ ID NOs. 48-55 or 59-70.

In some embodiments, the first alpha casein may be a truncated form of an alpha casein. In some embodiments, the first alpha casein comprises a N-terminal truncation of alpha-S1-casein protein. In some embodiments, the first alpha casein lacks 1 to 59 N-terminal amino acids of alpha-S1-casein protein. In some embodiments, the first alpha-S1-casein protein comprises a bovine amino acid sequence starting at amino acid 23, 24, 25 or 26 of SEQ ID NO. 3.

In some embodiments, the micellar form does not include a beta casein protein or a derivative thereof.

In some embodiments, the hybrid micelle comprises a beta casein protein. In some embodiments, the beta casein protein may be from the same species as the first alpha casein protein of the hybrid micelle composition. In some embodiments, the beta casein protein may be from the same species as the first kappa casein protein of the hybrid micelle composition. In some embodiments, the beta casein protein may be from a species that may be different from the first alpha casein protein and the first kappa casein protein of the hybrid micelle composition. In some embodiments, the beta casein protein may be selected from the group consisting of: a full-length beta casein protein, a gamma casein protein, and an alternate truncation of beta casein protein. In some embodiments, the beta casein protein may be a recombinant protein. In some embodiments, the first alpha casein protein may be not phosphorylated or may be substantially reduced in phosphorylation as compared to native alpha casein protein.

In some embodiments, the first alpha casein protein comprises a phosphorylation pattern that differs from native alpha casein protein. In some embodiments, the first kappa casein protein may be not glycosylated or may be substantially reduced in glycosylation as compared to native kappa casein protein. In some embodiments, the first kappa casein protein comprises a glycosylation pattern that differs from native kappa casein protein.

In some embodiments, the ratio of total alpha casein protein to total kappa casein protein in the composition may be about 1:1 to about 10:1 or about 3:1 to about 5:1.

In some embodiments, the first alpha casein protein, the first kappa casein protein or both the first alpha casein protein and the first kappa casein protein may be produced from a plant or a mammalian host cell.

In some embodiments, the first alpha casein protein, the first kappa casein protein or both the first alpha casein protein and the first kappa casein protein may be produced from a microbial host cell. In some embodiments, the microbial host cell may be selected from the group consisting of a bacteria, a yeast, and a fungus. In some embodiments, the microbial host cell may be a bacteria selected from the group consisting of Lactococci sp., Lactococcus lactis, Bacillus subtilis, Bacillus amyloliquefaciens, Bacillus licheniformis, Bacillus megaterium, Brevibacillus choshinensis, Mycobacterium smegmatis, Rhodococcus erythropolis and Corynebacterium glutamicum, Lactobacilli sp., Lactobacillus fermentum, Lactobacillus casei, Lactobacillus acidophilus, Lactobacillus plantarum, Synechocystis sp. 6803, and E. coli.

In some aspects, provided herein are protein powders comprising the hybrid micelle compositions described herein.

In some aspects, provided herein are colloids comprising the hybrid micelle compositions described herein.

In some aspects, provided herein are dairy-like product derived from the hybrid micelle compositions described herein.

In some embodiments, the hybrid micelle composition provides one or more dairy-like features selected from the group consisting of texture, melt, stretch, turbidity, and appearance.

In some embodiments, the dairy-like product may be selected from the group consisting of milk, cream, ice cream and yogurt. In some embodiments, the dairy-like product may be a cheese analogue. In some embodiments, the dairy-like product may be a coagulated colloid of the hybrid micelle composition. In some embodiments, the coagulated colloid may be cheese curd. In some embodiments, the coagulated colloid may be cheese. In some embodiments, the cheese may be a soft cheese, a hard cheese or an aged cheese. In some embodiments, the cheese may be selected from the group consisting of pasta-filata like cheese, paneer, cream cheese, and cottage cheese. In some embodiments, the cheese may be selected from the group consisting of mozzarella, cheddar, swiss, brie, camembert, feta, halloumi, gouda, edam, cheddar, manchego, swiss, Colby, muenster, blue cheese and parmesan. In some embodiments, the coagulated colloid may be yogurt. In some embodiments, the dairy-like product does not include any animal-sourced dairy protein. In some embodiments, the dairy-like product does not include any dairy-related protein other than caseins. In some embodiments, the dairy-like product may comprise at least one additional protein other than caseins. In some embodiments, the dairy-like product comprises at least one additional protein may be a dairy related protein other than caseins. In some embodiments, the dairy-related protein may be a whey protein.

In some aspects, provided herein are methods for producing a hybrid micelle composition. The method may comprise providing an alpha casein protein and a kappa casein protein. In some embodiments, the at least one of the alpha casein protein and the kappa casein protein may be a recombinant protein. In some embodiments, the alpha casein protein and the kappa casein protein may be from a different mammalian species. In some embodiments, the alpha casein protein comprises a deletion in the amino acid sequence as compared to a native alpha casein protein sequence. In some embodiments, the alpha casein protein and a kappa casein protein and at least one salt are combined under conditions wherein alpha casein protein and a kappa casein protein form a micellar form in a liquid colloid. In some embodiments, the salt may be selected from the group consisting of a calcium salt, a citrate salt, a phosphate salt, and any combination thereof.

In some embodiments, the micellar form further comprises a beta casein protein. In some embodiments, the micellar form lacks a beta casein protein. In some embodiments, the beta casein protein comprises a full-length beta casein protein, a gamma casein protein or an alternate truncation of beta casein protein.

In some embodiments, the method further comprises subjecting the liquid colloid to a first condition to form coagulates. In some embodiments, the first condition may be the addition of acid or acidification of the liquid colloid with a microorganism. In some embodiments, the method further comprises subjecting the coagulates to a hot water treatment and optionally stretching, to form a filata-type cheese.

In some embodiments, the method further comprises subjecting the coagulates to a renneting agent to form a rennetted curd. In some embodiments, the renneting agent may be a microbially-derived chymosin enzyme.

In some embodiments, the method further comprises aging and maturing the rennetted curd to form a cheese-like food product.

In some embodiments, the method further comprises subjecting the rennetted curd to a hot water treatment and optionally stretching, to form a filata-type cheese food product.

In some embodiments, the recombinantly produced alpha casein protein and/or kappa casein protein may be produced from a plant or a mammalian host cell.

In some embodiments, the recombinantly produced alpha casein protein and/or kappa casein protein may be produced from a microbial host cell. In some embodiments, the microbial host cell may be selected from the group consisting of a bacteria, a yeast, and a fungus. In some embodiments, the microbial host cell may be a bacteria selected from the group consisting of Lactococci sp., Lactococcus lactis, Bacillus subtilis, Bacillus amyloliquefaciens, Bacillus licheniformis, Bacillus megaterium, Brevibacillus choshinensis, Mycobacterium smegmatis, Rhodococcus erythropolis and Corynebacterium glutamicum, Lactobacilli sp., Lactobacillus fermentum, Lactobacillus casei, Lactobacillus acidophilus, Lactobacillus plantarum, Synechocystis sp. 6803, and E. coli.

In some embodiments, the method further comprises drying the liquid colloid to produce a micellar casein containing protein powder. In some embodiments, the drying comprises spray-drying, freeze-drying or drum drying.

In some embodiments, the method further comprises subjecting the liquid colloid to salt or acid precipitation to produce a caseinate-like protein powder.

In some aspects, provided herein are protein powders produced by the methods described herein. In some embodiments, the powder may be a spray-dried, freeze-dried or drum dried powder. In some aspects, provided herein are dairy-like products comprising the protein powders described herein. In some embodiments, the dairy-like product may be selected from the group consisting of milk, cream, ice-cream, yogurt, mozzarella cheese analogue, curd and cheese.

In some aspects, provided herein are hybrid micelle compositions. In some embodiments, the hybrid micelles comprise an alpha casein protein and a recombinant truncated kappa casein protein. In some embodiments, the alpha casein protein and the recombinant truncated kappa casein protein may be associated in micellar form. In some embodiments, the recombinant truncated kappa casein protein comprises a C-terminal truncation of a kappa casein protein amino acid sequence. In some embodiments, the kappa casein protein may be from a species selected from the group consisting of bovine, ovine and caprine. In some embodiments, the kappa casein protein comprises a bovine amino acid sequence truncated after amino acid 153 of SEQ ID NO. 54.

In some embodiments, the alpha casein protein may be a recombinant protein. In some embodiments, the alpha casein protein lacks one or more native post-translational modifications. In some embodiments, the alpha casein protein may be a native protein. In some embodiments, the hybrid micelle further comprises aggregates of the alpha casein protein and the recombinant truncated kappa casein protein. In some embodiments, the aggregates may be about or greater than 1000 nm in size. In some embodiments, the micelles may be in a size range of 300-610 nm, 300-530 nm, or 340-610 nm.

In some aspects, provided herein are dairy-like products comprising the hybrid micelle compositions described herein. In some embodiments, the dairy product may be a curd.

In some embodiments, the dairy product may be a cheese. In some embodiments, the cheese has a texture, mouthfeel, crumble, melt or any combination thereof desirable for a crumbly cheese, such as feta, asiago, blue, cheddar, cheshire, cotija, paneer.

In some aspects, provided herein is a pasta-filata cheese product. In some embodiments, the pasta-filata cheese product comprises between 70-85% of an alpha casein; between 15-30% of an ovine kappa casein. In some embodiments, the at least one of the alpha casein and the kappa casein is a recombinant protein. In some embodiments, the alpha casein and the kappa casein are associated in micellar form. In some embodiments, the pasta-filata cheese product comprises a fat in range 0-50%; and one or more salts from group of: calcium, phosphate and citrate. In some embodiments, the cheese product is able to melt and stretch. In some embodiments, the cheese product displays melt and stretch superior to cheese derived using bovine kappa casein, and wherein the cheese product has pasta-filata like structure and/or firmness.

In some embodiments, the pasta-filata cheese product comprises between 70-85% of an alpha casein and between 15-30% of a bovine or caprine kappa casein. In some embodiments, the at least one of the alpha casein and the kappa casein is a recombinant protein. In some embodiments, the alpha casein and the kappa casein are associated in micellar form. In some embodiments, the cheese product comprises a fat in range 0-50%; and one or more salts from group of: calcium, phosphate and citrate. In some embodiments, the cheese product has a pasta-filata-like melt, stretch, structure and/or firmness.

In some embodiments, the pasta-filata cheese product does not comprise any dairy-obtained components. In some embodiments, the at least one of the alpha casein and kappa casein is an altered form of the protein.

In some aspects, provided herein is a hard-matured cheese product. In some embodiments, the hard-matured cheese product comprises between 70-85% of an alpha casein and between 15-30% of an ovine kappa casein. In some embodiments, the alpha casein and the kappa casein are associated in a combination of micellar and aggregate forms. In some embodiments, the cheese product comprises a fat in range 0-50%; and one or more salts from group of: calcium, phosphate and citrate. In some embodiments, the cheese product has a swiss-like or cheddar-like consistency and is capable of melting.

In some embodiments, the hard-matured cheese product does not comprise any dairy-obtained components. In some embodiments, the at least one of the alpha casein and kappa casein is an altered form of the protein.

In some aspects, provided herein is a very hard dry cheese product. In some embodiments, the very hard cheese product comprises between 70-85% of an alpha casein and between 15-30% of an ovine kappa casein. In some embodiments, the alpha casein and the kappa casein are associated in a combination of micellar and aggregate forms; a fat in range 0-50%; and one or more salts from group of: calcium, phosphate and citrate. In some embodiments, the cheese product has a parmesan-like crumbly consistency and is capable of melting.

In some embodiments, the very hard dry cheese product does not comprise any dairy-obtained components. In some embodiments, the at least one of the alpha casein and kappa casein is an altered form of the protein.

In some aspects, provided herein is a goat-like soft and spreadable cheese product comprising: between 70-85% of an alpha casein lacking PTMs and between 15-30% of a caprine kappa casein. In some embodiments, the at least one of the alpha casein and the kappa casein is a recombinant protein. In some embodiments, the alpha casein and the kappa casein are associated in micellar form and the cheese comprises a fat in range 0-50%; and one or more salts from group of: calcium, phosphate and citrate. In some embodiments, the cheese product has a soft and spreadable consistency.

In some embodiments, the goat-like soft and spreadable cheese product does not comprise any dairy-obtained components. In some embodiments, the at least one of the alpha casein and kappa casein is an altered form of the protein.

In some aspects, provided herein is a soft cheese product. In some embodiments, the soft cheese product comprises between 70-75% of a truncated alpha casein and between % of a kappa casein. In some embodiments, the at least one of the alpha casein and the kappa casein is a recombinant protein. In some embodiments, the alpha casein and the kappa casein are associated in micellar form. In some embodiments, the cheese product comprises a fat in range 0-50%; and one or more salts from group of: calcium, phosphate and citrate. In some embodiments, the cheese product has a cottage-cheese like consistency and moisture.

In some embodiments, the soft cheese product does not comprise any dairy-obtained components. In some embodiments, the at least one of the alpha casein and kappa casein is an altered form of the protein.

In some aspects, provided herein is a yogurt-like product. In some embodiments, the yogurt-like product comprises between 70-75%% of a truncated alpha casein and between of a kappa casein. In some embodiments, the at least one of the alpha casein and the kappa casein is a recombinant protein. In some embodiments, the alpha casein and the kappa casein are associated in micellar form. In some embodiments, the cheese product comprises a a fat and a sugar, and one or more salts from group of: calcium, phosphate, citrate. In some embodiments, the yogurt-like product has a moist and loose consistency.

In some embodiments, the yogurt-like product does not comprise any dairy-obtained components. In some embodiments, the at least one of the alpha casein and kappa casein is an altered form of the protein.

In some aspects, provided herein is a crumbly cheese product. In some embodiments, the crumbly cheese product comprises between 70-75% of an alpha casein; between a 25-30% of a truncated bovine kappa casein. In some embodiments, the alpha casein and the kappa casein are associated in a combination of micellar and aggregate forms. In some embodiments, the cheese product comprises a fat in range 0-50%; and one or more salts from group of: calcium, phosphate and citrate. In some embodiments, the cheese product has a crumbly consistency and is capable of melting.

In some embodiments, the crumbly cheese product does not comprise any dairy-obtained components. In some embodiments, the at least one of the alpha casein and kappa casein is an altered form of the protein.

In some aspects, described herein are methods for regulating the yield of curd. The method comprises providing an alpha casein protein and a kappa casein protein. In some embodiments, the method comprises combining the alpha casein protein and a kappa casein protein and at least one salt under conditions wherein alpha casein protein and a kappa casein protein form a micellar form in a liquid colloid and coagulating the liquid colloid to form a curd. In some embodiments, the ratio of alpha casein protein to a kappa casein protein regulates the yield of curd. In some embodiments, the alpha casein protein and the kappa casein protein are from different species.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings.

FIG. 1 illustrates micelle and sub-micelle particle size distributions in nm for (left to right) hypophosphorylated bovine alpha casein+recombinant goat kappa casein, hypophosphorylated bovine alpha casein+bovine kappa casein (control sample), bovine alpha casein+recombinant goat kappa casein, bovine alpha casein+bovine kappa casein (control sample). Each sample is measured thrice. The dot in the plot represents a particle population of a particular size, and its color intensity corresponds to the proportion of that particle population among all particle populations detected.

FIG. 2 illustrates micelle and sub-micelle particle size distributions in log 10 (nm) for (left to right) unphosphorylated recombinant bovine alpha casein+recombinant goat kappa casein, unphosphorylated recombinant bovine alpha casein+bovine kappa casein (control sample). Each sample is measured thrice. The dot in the plot represents a particle population of a particular size, and its color intensity corresponds to the proportion of that particle population among all particle populations detected.

FIG. 3 illustrates micelle and sub-micelle particle size distributions in log 10 (nm) for (left to right) native bovine alpha casein+native bovine kappa casein, native bovine alpha casein+recombinant bovine kappa casein, native bovine alpha casein+recombinant goat kappa casein, native bovine alpha casein+recombinant sheep kappa casein, native bovine alpha casein+recombinant buffalo kappa casein. Each sample is measured thrice. The dot in the plot represents a particle population of a particular size, and its color intensity corresponds to the proportion of that particle population among all particle populations detected.

FIG. 4 illustrates micelle and sub-micelle particle size distributions in log 10 (nm) for (left to right) native bovine alpha casein+native bovine kappa casein, recombinant bovine alpha-S1-casein+native bovine kappa casein, recombinant bovine alpha-S1-casein+recombinant bovine kappa casein, recombinant bovine alpha-S1-casein+recombinant goat kappa casein, recombinant bovine alpha-S1-casein+recombinant sheep kappa casein. Each sample is measured thrice. The dot in the plot represents a particle population of a particular size, and its color intensity corresponds to the proportion of that particle population among all particle populations detected.

FIG. 5 illustrates micelle and sub-micelle particle size distributions in log 10 (nm) for (left to right) native bovine alpha casein+native bovine kappa casein, recombinant bovine alpha-S1-casein+native bovine kappa casein, recombinant 6×his-sheep alpha-S1-casein+native bovine kappa casein, recombinant bovine 6×his-alpha-S1-casein+native bovine kappa casein, recombinant goat 6×his-alpha-S1-casein+native bovine kappa casein. Each sample is measured thrice. The dot in the plot represents a particle population of a particular size, and its color intensity corresponds to the proportion of that particle population among all particle populations detected.

FIG. 6 illustrates micelle and sub-micelle particle size distributions in log 10 (nm) for (left to right) recombinant bovine alpha-S1-casein+recombinant goat kappa casein having 3:1, 4:1 and 5:1 alpha to kappa ratio respectively, and recombinant bovine alpha-S1-casein+recombinant sheep kappa casein having 3:1, 4:1 and 5:1 alpha to kappa ratio respectively. Each sample is measured thrice. The dot in the plot represents a particle population of a particular size, and its color intensity corresponds to the proportion of that particle population among all particle populations detected.

FIG. 7 illustrates cheese yields made from micelles having recombinant bovine alpha-S1-casein with recombinant goat kappa casein or recombinant sheep kappa casein with varying proportion of alpha to kappa casein (left to right) 3:1, 4:1 and 5:1 respectively.

FIG. 8 illustrates micelle and sub-micelle particle size distributions in log 10 (nm) for (left to right) native bovine alpha casein+native bovine kappa casein, recombinant bovine alpha-S1-casein+native bovine kappa casein, recombinant truncated bovine alpha-S1-casein+native bovine kappa casein, recombinant bovine alpha-S1-casein+recombinant sheep kappa casein, recombinant truncated bovine alpha-S1-casein+recombinant sheep kappa casein. Each sample is measured thrice. The dot in the plot represents a particle population of a particular size, and its color intensity corresponds to the proportion of that particle population among all particle populations detected.

FIG. 9 illustrates micelle and sub-micelle particle size distributions in log 10 (nm) for (left to right) native bovine alpha casein+native bovine kappa casein, native bovine alpha casein+recombinant bovine kappa casein, native bovine alpha casein+recombinant truncated bovine kappa casein, recombinant bovine alpha-S1-casein+native bovine kappa casein, recombinant bovine alpha-S1-casein+recombinant bovine kappa casein, and recombinant bovine alpha-S1-casein+recombinant truncated bovine kappa casein. Each sample is measured thrice. The dot in the plot represents a particle population of a particular size, and its color intensity corresponds to the proportion of that particle population among all particle populations detected.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Although the dairy industry is worth $330 billion, research needs to be performed for a clean dairy solution using recombinant dairy proteins. Replicating, mimicking or enhancing dairy-like properties of real dairy using plant-based ingredients has fallen short and majority of plant-based dairy-like products, especially yogurts and cheeses, show non-desirable product properties. They cannot achieve properties such as dairy-like melt, stretch and texture of cheese, or creaminess and mouthfeel of yogurts, milks and drinks. Presented herein are methods and compositions for recombinant dairy products with novel and controllable dairy-like properties.

A component that gives dairy cheese or yogurt its unique characteristics is the casein proteins that form micelles in milk. Micelles are protein colloids comprised of four casein proteins (alpha-s1-casein, alpha-s2-casein, beta casein, and kappa casein) that interact with insoluble calcium phosphate at the colloid centre. It is the micelles in milk that attract each other once chymosin is added to milk. This forms the curd, which is then used to make 99% of all cheeses. In case of yogurt, acidification of the micelle comprising liquid colloid may be performed using a starter culture of bacteria known for yogurt production. The current disclosure is based on the discovery that non-naturally occurring mixtures of caseins can be used to generate hybrid micelles. The hybrid micelles may be composed of casein proteins derived from different animal species, and in particular species from different sub-families for which crossing cannot happen in nature. Such hybrid micelles may provide properties that have similarities and/or differences from micelles occurring in nature and may be used for dairy-like products. Hybrid micelles also may be composed of caseins, where one or more of the caseins in the micelle is not a full-length casein, but instead is truncated.

Milk from different species differ from each other in various characteristics including flavour, nutrition value, protein content and micelle sizes. Protein content of milk from different species can also reflect the different growth rate of the neonate species, e.g., its requirements for essential amino acids. As such, the casein proteins found in each species show great inter-species diversity, especially in the presence of absence of alpha casein fractions. The coagulation and gel forming properties of the casein proteins also show diversity owing to the differences in the proteins. Even within the subfamily, where caseins may be similar, the properties of micelles, colloids derived from the micelles and consumables made using them are unpredictable and it has not been elucidated so far how this variability is regulated. For instance, sheep and goat milks differ substantially in their curds and cheeses even though their caseins are found to be highly similar with only 5-10 residue differences between the proteins.

Additionally, micelles produced by goat and sheep species have low levels of alpha casein, or in some cases, no alpha casein. Micelle properties also differ in species, for instance, in terms of micelle size, micelles of both goat and sheep are smaller (˜180 nm-190 nm for goat and sheep vs ˜260 nm for bovine), and have smaller sub-micellar structures present, than bovine micelles.

One skilled in the art, therefore, would not expect to derive micelles or especially, functional micelles with desirable properties and dairy-like products made using casein combinations from different species. The current disclosure is based on the surprising discovery made by the inventors that recombinantly produced caseins are able to form micelles when the micelles are comprised of casein proteins derived from different animal species and that such micelles provide functional properties for dairy-like products and ingredients. The current disclosure also describes dairy-like products such as cheese, milk, yogurt and other dairy products, as well as powders using the micelles formed by the methods described herein. Furthermore, the disclosure explains the discovery made by inventors that particular desired dairy product properties such as melt, stretch, crumbliness, creaminess or yield (moisture binding) can be directly controlled for by selection of the variants and amounts of caseins used in hybrid micelles-derived dairy-like products.

The current disclosure also describes hybrid micelles that incorporate truncated forms of casein proteins. While truncated forms of casein proteins such as alpha casein, beta casein and kappa casein are found in some curds and cheeses made using natural milk due to proteolysis, such caseins are no longer in a micelle form. One skilled in the art would not expect to generate stable micelles using truncated forms of casein proteins. Hybrid micelles described in the current disclosure can be formed using one or more truncated proteins. Compositions such as micelles described herein may comprise different truncated forms of casein proteins, found naturally or otherwise. Hybrid micelles formed with different truncated forms of casein proteins may be used to form consumable products such as cheeses in order to provide different qualities like flavour or consistency.

In some cases, hybrid micelles formed with truncated caseins may have different colloidal properties as compared to native micelles and can thus be used for specific dairy applications. For instance, a hybrid micelle comprising the bovine F24 alpha-S1-casein truncant (SEQ ID NO: 6) creates gels of higher moisture content, which are weaker and can be used to produce soft cheeses, spreadable cheeses, liquid dairy preparations and yogurt-like products.

The hybrid micelles described herein may be formed with one or more recombinant casein proteins. Recombinant casein protein may be expressed in a microbial or plant organism, for example, bacteria such as gram-positive bacteria Lactococcus lactis and Bacillus subtilis, as well as a gram-negative model organism E. coli, yeast such as Saccharomyces cerevisiae or Pichia pastoris, fungi such as Trichoderma reesei or Aspergillus niger, plant such as Glycine max (soy). These recombinant proteins may be combined with other components (e.g., minerals, fats, sugars, and vitamins) to make dairy-like products, for example cheese that behaves, smells, tastes, looks and feels like animal-obtained dairy cheese. Such dairy-like products may have no: i) lactose, ii) cholesterol, iii) saturated fats (depending on how it affects the taste and mouthfeel), and/or iv) whey proteins.

In some embodiments, the methods include producing recombinant protein in a bacterial host cell, such that such proteins are secreted from the cell into the surrounding media. In some embodiments, the methods include producing recombinant protein in a bacterial host cell, such that such proteins are intracellular. Recombinant protein can then be isolated, purified or partially purified and used in methods for making hybrid micelles, and products therefrom. Micelles can be dried and used as a dairy ingredient or emulsified with plant-based fats and other nutrients to form milk, cheese, yogurt or other dairy-like products.

In some embodiments, the methods include producing hybrid micelles using caseins from different species. The caseins may be from the species of the same genera and subfamilies or different genera, subfamilies, families or orders. Subfamilies and genera are based on animal classification and can include Bovinae (cattle, bison, buffalo), Caprinae (sheep and goat), Equine (horses, zebra) and Camelus (camels). Caseins may be altered as compared to native caseins, for instance, truncations of native caseins. Micelles may be produced with or without beta casein. Micelles generated using interspecies caseins may be of a different size than any of the parent species micelles or similar to one of the parent species micelles, may be rennetable under chymosin treatment with similar or different times as the parent species micelles, show different heat and pressure stability properties, different flavour profiles in dairy products and different allergenicity profiles to consumers allergic to dairy proteins.

In some embodiments, recombinant caseins can be isolated, purified or partially purified from genetically modified microorganisms or their cultivation broth.

The term “about” as used herein can mean within 1 or more than 1 standard deviation. Alternatively, “about” can mean a range of up to 10%, up to 5%, or up to 1% of a given value. For example, about can mean up to ±10%, ±9%, ±8%, ±7%, ±6%, ±5%, ±4%, ±3%, ±2%, or ±1% of a given value.

The term “dairy protein” as used herein means a protein that has an amino acid sequence derived from a protein found in milk (including variants thereof).

The term “animal-obtained dairy protein” as used herein means a protein obtained or derived from milk or a milk source (such as a caseinate made from milk), such as a protein obtained and/or isolated from a milk-producing organism, including but not limited to cow, sheep, goat, human, bison, buffalo, camel and horse. “Animal-obtained casein protein” means casein protein obtained and/or isolated from a milk-producing organism.

The term “recombinant dairy protein” as used herein means a protein that is expressed in a heterologous or recombinant organism that has an amino acid sequence derived from a protein found in milk (including variants thereof). “Recombinant casein protein” means a casein produced by a recombinant organism or in a heterologous host cell.

“Hybrid micelle” as used herein means a micelle formed from a mixture of two or more casein proteins, where at least one of the casein proteins is derived from a different animal species than the other casein protein(s) in the micelle, or where at least one of the alpha or kappa casein proteins is not a full-length casein protein. In some cases, at least one of the casein proteins in a hybrid micelle is a synthetic or recombinant protein. In some cases, all of the casein proteins in the hybrid micelle are recombinant proteins. In some cases, hybrid micelles comprise proteins other than casein proteins, for instance non-casein and non-dairy proteins.

“Liquid colloid” as used herein means a liquid comprising micelles, where the micelles are substantially in suspension within the liquid (e.g., where the micelles remain dispersed and do not settle out of the liquid solution). In some cases, the liquid colloid includes casein containing micelles and other forms of the caseins such as aggregates, submicelles and/or monomeric forms of the proteins.

“Derived from” or “from an animal species” as used in reference to a casein derived from animal species, derived from a different animal species, derived from a particular species (e.g., bovine, caprine etc.) means a casein protein that has an amino acid sequence that is identical to or having at least 90% sequence identity with a casein protein found in nature from the animal species. In some cases, a casein protein derived from an animal species has at least 91%, 92%, 93%, 94%, 95%, 96%, 97%, 98%, 99%, 99.5% or 100% sequence identity with a casein protein found in nature.

A percentage of “sequence identity” as used herein in the context of polynucleotide or polypeptide (amino acid) sequences refers to the percentage of residues in two sequences that are the same when the sequences are aligned for maximum correspondence. There are a number of different algorithms known in the art that can be used to measure polynucleotide or polypeptide sequence identity. For instance, sequences can be compared using FASTA (e.g., using its default parameters as provided in the Wisconsin Package Version 10.0, Genetics Computer Group (GCG), Madison, WI), Gap (e.g., using its default parameters as provided in the Wisconsin Package Version 10.0, GCG, Madison, WI), Bestfit, ClustalW (e.g., using default parameters of Version 1.83), or BLAST (e.g., using reciprocal BLAST, PSI-BLAST, BLASTP, BLASTN) (see, for example, Pearson. 1990. Methods Enzymol. 183:63; Altschul et al. 1990. J. Mol. Biol. 215:403).

### Compositions of Hybrid Micelles and Methods of Making Compositions

I. Hybrid Micelles Derived from Different Animal Species Casein Proteins

In mammalian milk, casein proteins (alpha-s1-casein, alpha-s2-casein, beta casein, and kappa casein, and a cleaved form of beta casein called gamma casein) and calcium phosphate and citrate form large colloidal particles called casein micelles. The main function of the casein micelle is to provide fluidity to casein molecules and solubilize phosphate and calcium. In nature, milk contains some form or combination of alpha, kappa and beta caseins.

As described herein, micelles are protein particles comprising one or more casein proteins. Micelles, including sub-micelles, may include particles with a diameter from 10 nm to 900 nm. Micelles may be formed in a liquid colloid, for instance in a liquid colloid comprising one or more casein proteins. A population of micelles formed using one or more casein molecules may comprise micelles of various sizes. Micelles may be in a solution or in a dehydrated form. Casein micelles may be formed with isolated casein proteins, such as recombinantly produced casein protein. Micelles formed from recombinant casein may include either alpha casein, such as alpha-s1-casein and/or alpha-s2-casein, beta casein and/or kappa casein. In some cases, micelles comprise alpha casein and kappa casein. In some cases, micelles comprise alpha casein and kappa casein, and do not contain any beta casein protein.

In some embodiments, the methods include producing micelles using caseins derived from different animal species and may be referred to as hybrid micelles. The caseins may be derived from the same subfamilies or different subfamilies. One or more caseins in a hybrid micelle composition may be produced recombinantly. A hybrid micelle described herein may be comparable to a naturally found micelle (or a non-hybrid micelle comprising caseins from the same species) in size, micellar (particle) charge, stability over time, and/or stability upon heat and pressure treatment. A hybrid micelle composition may have functional properties naturally formed micelles have such as coagulation upon acidification, coagulation upon renneting, curd properties (cohesiveness, yield) as a function of micellar function, cheese properties (moisture, yield, melt, stretch) as a function of micellar function and such properties may be comparable to a naturally found micelle (or a non-hybrid micelle comprising caseins from the same species). Alternatively, properties of hybrid micelles such as coagulation upon acidification, coagulation upon renneting, curd properties (cohesiveness, yield) as a function of micellar function, cheese properties (moisture, yield, melt, stretch) as a function of micellar function and such properties may be different from a naturally found micelle (or a non-hybrid micelle comprising caseins from the same species) but may still be able to form dairy-like products with desired properties.

In some cases, a hybrid micelle composition comprises an alpha casein, a beta casein and a kappa casein. Alternatively, a hybrid micelle may be formed without a beta casein. Alpha casein as described herein may comprise alpha-S1-casein or alpha-S2-casein, or both. A hybrid micelle may comprise at least one casein protein from a species different from the other casein proteins. For instance, in some cases, an alpha casein in a hybrid micelle composition from species A may be combined with a beta and kappa casein from species B to form a hybrid micelle. In some cases, alpha and beta caseins from species A may be combined with kappa casein from species B to form a hybrid micelle. In some cases, alpha and kappa caseins from species A may be combined with a beta casein from species B to form a hybrid micelle. In some cases, alpha casein from species A may be combined with a beta casein from species B and a kappa casein from species C to form a hybrid micelle. In some cases, alpha casein protein in a hybrid micelle may be a combination of alpha casein proteins from species A and B. In some cases, beta casein protein in a hybrid micelle may be a combination of beta casein proteins from species A and B. In some cases, kappa casein protein in a hybrid micelle may be a combination of kappa casein proteins from species A and B. In some cases, a hybrid micelle comprises alpha casein protein and kappa casein protein, without beta casein protein, and the alpha casein protein is from species A and the kappa casein protein is from species B. In some cases, a hybrid micelle comprises alpha casein protein and kappa casein protein, without beta casein protein, and the alpha casein protein and/or the kappa casein protein may be a combination of such casein from species A and B.

In some cases, a hybrid micelle comprises kappa casein from species A and kappa casein from species B, without alpha and beta casein proteins. In some cases, a hybrid micelle comprises alpha casein from species A and alpha casein from species B, without beta and kappa casein proteins. In some cases, a hybrid micelle comprises beta casein from species A and beta casein from species B, without alpha and kappa casein proteins.

In some cases, a micelle composition may be a hybrid micelle comprising a bovine alpha casein protein. The bovine alpha casein protein may be a recombinantly produced bovine alpha casein protein. The bovine alpha casein protein may be a truncated bovine alpha casein protein. The bovine alpha casein protein in a hybrid micelle may comprise a full-length bovine alpha casein protein and one or more truncated bovine alpha casein proteins. A hybrid micelle composition comprising a bovine alpha casein protein may comprise a recombinant kappa casein protein. A hybrid micelle composition comprising a bovine alpha casein protein may comprise an ovine kappa casein protein. A hybrid micelle composition comprising a bovine alpha casein protein may comprise a caprine kappa casein protein. A hybrid micelle composition comprising a bovine alpha casein protein may comprise an equine kappa casein protein. A hybrid micelle composition comprising a bovine alpha casein protein may comprise a camel kappa casein protein. A hybrid micelle composition comprising a bovine alpha casein protein may comprise a different mammalian kappa casein protein. A hybrid micelle composition comprising a bovine alpha casein protein may comprise a beta casein protein. The beta casein protein may be a bovine casein protein, wherein the kappa casein is from a different species or subfamily. Alternatively, the beta casein protein may be of the same species as the kappa casein protein, but from a species different than the alpha casein protein. Alternatively, a hybrid micelle composition comprising a bovine alpha casein protein may comprise a bovine kappa casein protein and comprises a beta casein protein from another species.

In some cases, a micelle composition may be a hybrid micelle comprising an ovine alpha casein protein. The ovine alpha casein protein may be a recombinantly produced ovine alpha casein protein. The ovine alpha casein protein may be a truncated ovine alpha casein protein. The ovine alpha casein protein in a hybrid micelle may comprise a full-length ovine alpha casein protein and one or more truncated ovine alpha casein proteins. A hybrid micelle composition comprising an ovine alpha casein protein may comprise a bovine kappa casein protein. A hybrid micelle composition comprising an ovine alpha casein protein may comprise a caprine kappa casein protein. A hybrid micelle composition comprising an ovine alpha casein protein may comprise an equine kappa casein protein. A hybrid micelle composition comprising an ovine alpha casein protein may comprise a camel kappa casein protein. A hybrid micelle composition comprising an ovine alpha casein protein may comprise a different mammalian kappa casein protein. A hybrid micelle composition comprising an ovine alpha casein protein may comprise a truncated kappa casein protein. A hybrid micelle composition comprising an ovine alpha casein protein may comprise a beta casein protein. The beta casein protein may be an ovine casein protein, wherein the kappa casein is from a different species or subfamily. Alternatively, the beta casein protein may be of the same species as the kappa casein protein, but from a species different than the alpha casein protein. Alternatively, a hybrid micelle composition comprising an ovine alpha casein protein may comprise an ovine kappa casein protein and comprises a beta casein protein from another species.

In some cases, a micelle composition may be a hybrid micelle comprising a caprine alpha casein protein. The caprine alpha casein protein may be a recombinantly produced caprine alpha casein protein. The caprine alpha casein protein may be a truncated caprine alpha casein protein. The caprine alpha casein protein in a hybrid micelle may comprise a full-length bovine alpha casein protein and one or more truncated caprine alpha casein proteins. A hybrid micelle composition comprising a caprine alpha casein protein may comprise a recombinant kappa casein protein. A hybrid micelle composition comprising a caprine alpha casein protein may comprise a bovine kappa casein protein. A hybrid micelle composition comprising a caprine alpha casein protein may comprise an ovine kappa casein protein. A hybrid micelle composition comprising a caprine alpha casein protein may comprise an equine kappa casein protein. A hybrid micelle composition comprising a caprine alpha casein protein may comprise a camel kappa casein protein. A hybrid micelle composition comprising a caprine alpha casein protein may comprise a different mammalian kappa casein protein. A hybrid micelle composition comprising a caprine alpha casein protein may comprise a truncated kappa casein protein. A hybrid micelle composition comprising a caprine alpha casein protein may comprise a beta casein protein. The beta casein protein may be a caprine casein protein, wherein the kappa casein is from a different species or subfamily. Alternatively, the beta casein protein may be of the same species as the kappa casein protein, but from a species different than the alpha casein protein. Alternatively, a hybrid micelle composition comprising a caprine alpha casein protein may comprise a caprine kappa casein protein and comprises a beta casein protein from another species.

In some cases, a hybrid micelle composition comprises casein proteins from two or more species or subfamilies. Subfamilies may include Bovinae (cattle, bison, buffalo), Caprinae (sheep and goat), Equine (horses, zebra) or Camelus (camels). In some cases, a hybrid micelle composition may include one or more casein proteins from a mammalian species. In some cases, a hybrid micelle composition may include one or more casein proteins from a ruminant species.

In some cases, a hybrid micelle composition comprises an alpha casein with at least 90% sequence identity to a sequence selected from SEQ ID NOs: 1-47. The hybrid micelle composition may comprise a beta casein with at least 90% sequence identity to a sequence selected from SEQ ID NOs: 71-85. The hybrid micelle composition may comprise a kappa casein with at least 90% sequence identity to a sequence selected from SEQ ID NOs: 48-70.

### II. Casein Protein Forms in Hybrid Micelles

In some embodiments, a hybrid micelle comprises casein protein, where one of the alpha or kappa casein proteins is an altered form of the casein protein, such as a truncated form. In some cases, the hybrid micelle containing such altered form of a casein protein comprises casein proteins from the same species and in some cases, they are from different species.

Alpha Casein: Alpha casein protein in a hybrid micelle may be an altered form of an alpha casein protein relative to a native alpha casein protein or an alpha casein protein in a hybrid micelle may comprise the same amino acid sequence as an alpha casein protein found in nature (also referred to herein as a non-altered alpha casein). Alpha casein protein in a hybrid micelle may be a mixture of an altered form of an alpha casein from one species and a non-altered alpha casein from another species. Alpha casein protein in a hybrid micelle may be a mixture of an altered form of an alpha casein from one species and a non-altered alpha casein from that same species. An altered form of an alpha casein protein may comprise one or more amino acid insertions, deletions, or substitutions relative to a wild-type or native alpha casein protein. An altered form of an alpha casein protein may be a truncated alpha casein protein relative to a wild-type or native alpha casein protein. The truncation may be a truncation found in nature or an engineered truncation. An altered form of an alpha casein protein may have a N-terminal truncation relative to a wild-type or native alpha casein protein. An altered form of an alpha casein protein may have a C-terminal truncation relative to a wild-type or native alpha casein protein.

In some cases, the alpha-S1-casein protein in a hybrid micelle is a truncated alpha-S1-casein protein relative to a wild-type or native alpha-S1-casein protein. In some cases, the alpha-S1-casein protein has a N-terminal truncation relative to a wild-type or native alpha-S1-casein protein. In some cases, the alpha-S1-casein protein has a C-terminal truncation relative to a wild-type or native alpha-S1-casein protein. In some cases, the alpha-S1-casein protein lacks 1 to 59 N-terminal amino acids relative to a wild-type or native alpha-S1-casein protein. In some cases, the alpha-S1-casein protein lacks 1 to 5, 1 to 10, 1 to 20, 1 to 30, 1 to 50, or 1 to 59 N-terminal amino acids relative to a wild-type or native alpha-S1-casein protein. In some cases, a bovine alpha-S1-casein protein lacks 1 to 59 N-terminal amino acids relative to a wild-type or native bovine alpha-S1-casein protein. In some cases, a bovine alpha-S1-casein protein with SEQ ID NO: 2 lacks 22, 23, 24, 25 or 59 N-terminal amino acids relative to a wild-type or native bovine alpha-S1-casein protein. In some cases, an alpha-S1 protein lacks between 1 to 59 N-terminal amino acids or lacks 22, 23, 24, 25 or 59 N-terminal amino acids of the alpha-S1-casein amino acid sequence that correspond to sequences in SEQ ID NO: 4-11 or 86.

Beta Casein: Beta casein protein in a hybrid micelle may be an altered form of a beta casein protein relative to a native beta casein protein or a beta casein protein in a hybrid micelle comprise the same amino acid sequence as a beta casein protein found in nature (also referred to herein as a non-altered beta casein). In some cases, a micelle may be formed using a mixture of beta casein proteins including an altered form of a beta casein protein and a non-altered beta casein protein. An altered form of a beta casein protein may comprise one or more amino acid insertions, deletions, or substitutions relative to a wild-type or native beta casein protein. An altered form of a beta casein protein may be a truncated beta casein protein relative to a wild-type or native beta casein protein. An altered form of a beta casein protein may have a N-terminal truncation relative to a wild-type or native beta casein protein. An altered form of a beta casein protein may have a C-terminal truncation relative to a wild-type or native beta casein protein.

Kappa Casein: Kappa casein protein in a hybrid micelle may be an altered form of a kappa casein protein relative to a native kappa casein protein or a kappa casein protein in a hybrid micelle comprise the same amino acid sequence as a kappa casein protein found in nature (also referred to herein as a non-altered kappa casein). In some cases, a micelle may be formed using a mixture of kappa casein proteins including an altered form of a kappa casein protein and a non-altered kappa casein protein. An altered form of a kappa casein protein may comprise one or more amino acid insertions, deletions, or substitutions relative to a wild-type or native kappa casein protein. An altered form of a kappa casein protein may be a truncated kappa casein protein relative to a wild-type or native kappa casein protein. An altered form of a kappa casein protein may have a N-terminal truncation relative to a wild-type or native kappa casein protein. An altered form of a kappa casein protein may have a C-terminal truncation relative to a wild-type or native kappa casein protein.

In some cases, a truncation in the kappa casein protein comprises a deletion in the amino acid sequence relative to the native kappa casein protein sequence. In some cases, the kappa casein protein lacks 1 to 27 C-terminal amino acids relative to the native kappa casein protein. In some cases, the kappa casein protein lacks 1 to 5, 1 to 10, 1 to 20, 1 to 25 or 1 to 27 C-terminal amino acids relative to the native kappa casein protein. In some cases, a bovine kappa casein protein sequence may be truncated after amino acid 142, 146 or 153 relative to the native bovine kappa casein protein sequence. In some cases, a bovine kappa casein protein with SEQ ID NO: 49 lacks 1 to 16 C-terminal amino acids. In some cases, a kappa casein protein sequence may lack between 1 to 27 C-terminal amino acids or may be truncated after an amino acid position that corresponds to amino acid 142, 146 or 153 relative to the native bovine kappa casein protein sequence.

### Recombinant Forms and Post-Translational Modifications (PTMs)

The alpha casein protein (comprising alpha-S1 and/or alpha-S2-caseins in non-altered or altered form) in a hybrid micelle may be produced recombinantly. In some cases, hybrid micelles may comprise only recombinantly produced alpha casein protein. In certain cases, hybrid micelles may comprise substantially only recombinantly produced alpha casein protein. For instance, alpha casein proteins may be 90%, 92%, 95%, 97%, or 99% recombinant alpha casein. Alternatively, hybrid micelles may comprise a mixture of recombinantly produced and animal-obtained alpha casein proteins.

Depending on the host organism used to express the alpha casein, the alpha casein proteins may have a glycosylation or phosphorylation pattern (e.g., post-translational modifications) different from animal-obtained alpha casein proteins. In some cases, the alpha casein protein comprises no post translational modifications (PTMs). In some cases, the alpha casein protein comprises substantially reduced PTMs as compared to the amount of PTMs found in an animal-obtained alpha casein protein, for example, at least 50%, at least 55%, at least 60%, at least 65%, at least 70%, at least 75%, at least 80%, at least 85%, at least 90%, at least 92%, at least 95%, at least 97%, or at least 99% less PTMs as compared to the amount of PTMs found in an animal-obtained alpha casein protein. Alternatively, the alpha casein protein may comprise PTMs comparable to animal-obtained alpha casein PTMs. In some cases, the alpha casein protein comprises substantially increased PTMs as compared to the amount of PTMs found in an animal-obtained alpha casein protein, for example, at least 5%, at least 10%, at least 20%, at least 30%, at least 40%, at least 50%, at least 60%, at least 70%, at least 80%, at least 90%, at least 95%, at least 97%, or at least 99% more PTMs as compared to the amount of PTMs found in an animal-obtained alpha casein protein.

The PTMs in the alpha casein protein may be modified chemically or enzymatically. In some cases, the alpha casein protein comprises substantially reduced or no PTMs without chemical or enzymatic treatment. Hybrid micelles may be generated using alpha casein protein with reduced or no PTMs, wherein the lack of PTMs is not due to chemical or enzymatic treatments of the protein, such as producing an alpha casein protein through recombinant production where the recombinant protein lacks PTMs.

The phosphorylation in the alpha casein protein may be modified chemically or enzymatically. In some cases, the alpha casein protein comprises substantially reduced or no phosphorylation without chemical or enzymatic treatment. For instance, alpha casein proteins may be 50%, 55%, 60%, 65%, 70%, 75%, 80%, 85%, 90%, 92%, 95%, 97%, 99% less phosphorylated as compared to animal-obtained alpha casein. In some aspects, the alpha casein protein is expressed recombinantly and the resulting alpha casein protein comprises substantially reduced or no phosphorylation. Hybrid micelles may be generated using alpha casein protein with reduced or no phosphorylation, wherein the lack of phosphorylation is not due to chemical or enzymatic treatments, such as where recombinant production provides alpha casein protein with reduced or no phosphorylation

The beta casein protein (or a beta casein truncant such as gamma casein) in hybrid micelles may be produced recombinantly. In some cases, hybrid micelles may comprise only recombinantly produced beta casein protein. In certain cases, hybrid micelles may comprise substantially only recombinantly produced beta casein protein. For instance, beta casein proteins may be at least 90%, at least 92%, at least 95%, at least 97%, at least 99% recombinant beta casein. Alternatively, hybrid micelles may comprise a mixture of recombinantly produced and animal-obtained beta casein proteins.

Depending on the host organism used to express the beta casein protein, the beta casein proteins may have a glycosylation or phosphorylation pattern (e.g., post-translational modifications) different from animal-obtained beta casein proteins. In some cases, the beta casein protein comprises no post translational modifications (PTMs). In some cases, the beta casein protein comprises substantially reduced PTMs as compared to the amount of PTMs found in an animal-obtained beta casein protein, for example, at least 50%, at least 55%, at least 60%, at least 65%, at least 70%, at least 75%, at least 80%, at least 85%, at least 90%, at least 92%, at least 95%, at least 97%, or at least 99% less PTMs as compared to the amount of PTMs found in an animal-obtained beta casein protein. Alternatively, the beta casein protein may comprise PTMs comparable to animal-obtained beta casein PTMs. In some cases, the beta casein protein comprises substantially increased PTMs as compared to the amount of PTMs found in an animal-obtained beta casein protein, for example, at least 5%, at least 10%, at least 20%, at least 30%, at least 40%, at least 50%, at least 60%, at least 70%, at least 80%, at least 90%, at least 95%, at least 97%, or at least 99% more PTMs as compared to the amount of PTMs found in an animal-obtained beta casein protein.

The PTMs in the beta casein protein may be modified chemically or enzymatically. In some cases, the beta casein protein comprises substantially reduced or no PTMs without chemical or enzymatic treatment. Hybrid micelles may be generated using beta casein protein with reduced or no PTMs, wherein the lack of PTMs is not due to chemical or enzymatic treatments of the protein, such as producing a beta casein protein through recombinant production where the recombinant protein lacks PTMs.

The phosphorylation in the beta casein protein may be modified chemically or enzymatically. In some cases, the beta casein protein comprises substantially reduced or no phosphorylation without chemical or enzymatic treatment. For instance, beta casein proteins may be at least 50%, at least 55%, at least 60%, at least 65%, at least 70%, at least 75%, at least 80%, at least 85%, at least 90%, at least 92%, at least 95%, at least 97%, or at least 99% less phosphorylated as compared to animal-obtained beta casein. Hybrid micelles may be generated using beta casein protein with reduced or no phosphorylation, wherein the lack of phosphorylation is not due to chemical or enzymatic treatments, such as where recombinant production provides beta casein protein with reduced or no phosphorylation.

The kappa casein protein (in altered or non-altered form) in hybrid micelles may be produced recombinantly. In some cases, hybrid micelles may comprise only recombinantly produced kappa casein protein. In certain cases, hybrid micelles may comprise substantially only recombinantly produced kappa casein protein. In some cases, kappa casein proteins may be at least 90%, at least 92%, at least 95%, at least 97%, or at least 99% recombinant kappa casein. Alternatively, hybrid micelles may comprise a mixture of recombinantly produced and animal-obtained kappa casein proteins.

Depending on the host organism used to express the kappa casein protein, the kappa casein proteins may have a posttranslational modification, such as glycosylation or phosphorylation pattern, different from animal-obtained kappa casein protein. In some cases, the kappa casein protein in the composition herein comprises no post translational modifications (PTMs). In some cases, the kappa casein protein comprises substantially reduced PTMs as compared to the amount of PTMs found in an animal-obtained kappa casein protein, for example, at least 50%, at least 55%, at least 60%, at least 65%, at least 70%, at least 75%, at least 80%, at least 85%, at least 90%, at least 92%, at least 95%, at least 97%, or at least 99% less PTMs as compared to the amount of PTMs found in an animal-obtained kappa casein protein. Alternatively, the kappa casein protein may comprise PTMs comparable to animal-obtained kappa casein PTMs. In some cases, the kappa casein protein comprises substantially increased PTMs as compared to the amount of PTMs found in an animal-obtained kappa casein protein, for example, at least 5%, at least 10%, at least 20%, at least 30%, at least 40%, at least 50%, at least 60%, at least 70%, at least 80%, at least 90%, at least 95%, at least 97%, or at least 99% more PTMs as compared to the amount of PTMs found in an animal-obtained kappa casein protein.

The PTMs in the kappa casein protein may be modified chemically or enzymatically. In some cases, the kappa casein protein comprises substantially reduced or no PTMs without chemical or enzymatic treatment. Hybrid micelles may be generated using kappa casein protein with reduced or no PTMs, wherein the lack of or reduction of PTMs is not due to chemical or enzymatic treatments, such as by producing recombinant kappa casein protein in a host where the kappa casein protein is not post-translationally modified or the level of PTMs is substantially reduced.

The glycosylation in the kappa casein protein may be modified chemically or enzymatically. In some cases, the kappa casein protein comprises substantially reduced or no glycosylation without chemical or enzymatic treatment. For instance, kappa casein proteins may be at least 50%, at least 55%, at least 60%, at least 65%, at least 70%, at least 75%, at least 80%, at least 85%, at least 90%, at least 92%, at least 95%, at least 97%, or at least 99% less glycosylated as compared to animal-obtained kappa casein protein. Hybrid micelles may be generated using kappa casein protein with reduced or no glycosylation, wherein the lack of glycosylation is not due to chemical or enzymatic treatments post recombinant production, such as by producing recombinant kappa protein in a host where the kappa casein protein is not glycosylated or the level of glycosylation is substantially reduced.

The phosphorylation in the kappa casein protein may be modified chemically or enzymatically. In some cases, the kappa casein protein comprises substantially reduced or no phosphorylation without chemical or enzymatic treatment. For instance, kappa casein proteins may be at least 50%, at least 55%, at least 60%, at least 65%, at least 70%, at least 75%, at least 80%, at least 85%, at least 90%, at least 92%, at least 95%, at least 97%, or at least 99% less phosphorylated as compared to animal-obtained kappa casein protein. Hybrid micelles may be generated using kappa casein protein with reduced or no phosphorylation, wherein the lack of phosphorylation is not due to chemical or enzymatic treatments, such as by producing recombinant kappa protein in a host where the kappa casein protein is not phosphorylated or the level of phosphorylation is substantially reduced.

### III. Hybrid Micelle Features

Hybrid micelle diameters herein may be from about 10 nm to 900 nm. Hybrid micelle diameters herein may be from about at least 10 nm. Hybrid micelle diameters herein may be from about at most 900 nm. Hybrid micelle diameters herein may be from about 10 nm to 50 nm, 10 nm to 100 nm, 10 nm to 200 nm, 10 nm to 300 nm, 10 nm to 400 nm, 10 nm to 500 nm, 10 nm to 600 nm, 10 nm to 700 nm, 10 nm to 800 nm, 10 nm to 900 nm, 50 nm to 100 nm, 50 nm to 200 nm, 50 nm to 300 nm, 50 nm to 400 nm, 50 nm to 500 nm, 50 nm to 600 nm, nm to 700 nm, 50 nm to 800 nm, 50 nm to 900 nm, 100 nm to 200 nm, 100 nm to 300 nm, 100 nm to 400 nm, 100 nm to 500 nm, 100 nm to 600 nm, 100 nm to 700 nm, 100 nm to 800 nm, 100 nm to 900 nm, 200 nm to 300 nm, 200 nm to 400 nm, 200 nm to 500 nm, 200 nm to 600 nm, 200 nm to 700 nm, 200 nm to 800 nm, 200 nm to 900 nm, 300 nm to 400 nm, 300 nm to 500 nm, 300 nm to 600 nm, 300 nm to 700 nm, 300 nm to 800 nm, 300 nm to 900 nm, 400 nm to 500 nm, 400 nm to 600 nm, 400 nm to 700 nm, 400 nm to 800 nm, 400 nm to 900 nm, 500 nm to 600 nm, 500 nm to 700 nm, 500 nm to 800 nm, 500 nm to 900 nm, 600 nm to 700 nm, 600 nm to 800 nm, 600 nm to 900 nm, 700 nm to 800 nm, 700 nm to 900 nm, or 800 nm to 900 nm. Hybrid micelle diameters herein may be from about 10 nm, 50 nm, 100 nm, 200 nm, 300 nm, 400 nm, 500 nm, 600 nm, 700 nm, 800 nm, or 900 nm. Hybrid micelle diameters herein may be at least 10 nm, 20 nm, 50 nm, 100 nm, 150 nm, 200 nm, 250 nm, 300 nm, 350 nm, 400 nm, 450 nm, 500 nm, 600 nm, 700 nm or 800 nm. Hybrid micelle diameters herein may be at most 20 nm, 50 nm, 100 nm, 150 nm, 200 nm, 250 nm, 300 nm, 350 nm, 400 nm, 450 nm, 500 nm, 600 nm, 700 nm, 800 nm or 900 nm.

The average diameter of micelles in a population of hybrid micelles may be from nm to 500 nm. The average diameter of micelles in a population of hybrid micelles may be at least 50 nm. The average diameter of micelles in a population of hybrid micelles may be at most 500 nm. The average diameter of micelles in a population of hybrid micelles may be from nm to 100 nm, 50 nm to 200 nm, 50 nm to 300 nm, 50 nm to 400 nm, 50 nm to 500 nm, 100 nm to 200 nm, 100 nm to 300 nm, 100 nm to 400 nm, 100 nm to 500 nm, 200 nm to 300 nm, 200 nm to 400 nm, 200 nm to 500 nm, 300 nm to 400 nm, 300 nm to 500 nm, or 400 nm to 500 nm. The average diameter of micelles in a population of hybrid micelles may be 50 nm, 100 nm, 200 nm, 300 nm, 400 nm, or 500 nm.

In some cases, the average diameter of micelles in a population of hybrid micelles may be larger. The average diameter of micelles in a population of hybrid micelles may be 500 nm to 900 nm. The average diameter of micelles in a population of hybrid micelles may be at least 500 nm. The average diameter of micelles in a population of hybrid micelles may be at most 900 nm. The average diameter of micelles in a population of hybrid micelles may be 500 nm to 600 nm, 500 nm to 700 nm, 500 nm to 800 nm, 500 nm to 900 nm, 600 nm to 700 nm, 600 nm to 800 nm, 600 nm to 900 nm, 700 nm to 800 nm, 700 nm to 900 nm, or 800 nm to 900 nm. The average diameter of micelles in a population of hybrid micelles may be about 500 nm, 600 nm, 700 nm, 800 nm, or 900 nm.

Micelle diameters may sometimes be dependent on the caseins used to produce the hybrid micelles. For instance, in some cases, a hybrid micelle with a bovine alpha casein and a goat kappa casein may have a different micelle diameter as compared to a hybrid micelle with bovine alpha casein and a sheep kappa casein. In some examples, a post translational modification pattern of a casein may alter the micelle diameters. For instance, a reduction in the phosphorylation of a casein (as compared to the casein in its native form) may lead to a larger micelle size as compared to a native micelle from a dairy source or a hybrid micelle with a native phosphorylation pattern.

Liquid colloid solutions as described herein may comprise one or more micelles in a solution. A liquid colloid may be formed when stable micelles comprising one or more casein particles are dispersed in a liquid medium. A dehydrated population of micelles may be reconstituted in a solvent to produce a liquid colloid. In some cases, the solvent may be water or alternatively, the solvent may be a salt solution.

In some cases, micelles include an alpha casein protein, such as alpha-S1 and/or alpha-S2 casein protein and kappa casein protein or an alpha casein protein with beta and kappa casein proteins. The ratio of alpha casein protein to kappa casein protein in the micelle may be about 2:1 to 10:1 or about 1:1 to 15:1. The ratio of beta casein protein to kappa-casein protein in the micelle may be about 2:1 to 10:1 or about 1:1 to 15:1.

In some embodiments, micelles described herein include micelles formed in a liquid solution. In some embodiments, casein containing micelles are present in a liquid colloid, where the micelles remain dispersed and do not settle out of the liquid solution. In some cases, the liquid colloid includes casein containing micelles and other forms of the caseins such as aggregates and/or monomeric forms of the proteins.

Casein content of hybrid micelles can be the total protein content of the hybrid micelle. Casein content of hybrid micelles can be 70-100% w/w of the total protein content of the hybrid micelle. Casein content of hybrid micelles can be 70%, 80%, 90% or 100% w/w of the total protein content of the hybrid micelle.

The casein content of hybrid micelles or hybrid micelle compositions may comprise from 1-10% (w/w) alpha casein, 1-10% (w/w) beta casein and 80-98% (w/w) kappa casein. The casein content of hybrid micelles or hybrid micelle compositions may comprise from 10-15% (w/w) alpha casein, 1-10% (w/w) beta casein and 75-89% (w/w) kappa casein. The casein content of hybrid micelles or hybrid micelle compositions may comprise from 15-25% (w/w) alpha casein, 1-10% (w/w) beta casein and 65-84% (w/w) kappa casein. The casein content of hybrid micelles or hybrid micelle compositions may comprise from 25-35% (w/w) alpha casein, 1-10% (w/w) beta casein and 55-74% (w/w) kappa casein. The casein content of hybrid micelles or hybrid micelle compositions may comprise from 35-45% (w/w) alpha casein, 1-10% (w/w) beta casein and 45-64% (w/w) kappa casein. The casein content of hybrid micelles or hybrid micelle compositions may comprise from 45-55% (w/w) alpha casein, 1-10% (w/w) beta casein and 45-64% (w/w) kappa casein. The casein content of hybrid micelles or hybrid micelle compositions may comprise from 55-65% (w/w) alpha casein, 1-10% (w/w) beta casein and 35-54% (w/w) kappa casein. The casein content of hybrid micelles or hybrid micelle compositions may comprise from 65-75% (w/w) alpha casein, 1-10% (w/w) beta casein and (w/w) kappa casein. The casein content of hybrid micelles or hybrid micelle compositions may comprise from 75-85% (w/w) alpha casein, 1-10% (w/w) beta casein and (w/w) kappa casein.

The casein content of hybrid micelles or hybrid micelle compositions may comprise from 1-10% (w/w) alpha casein, 10-30% (w/w) beta casein and 60-89% (w/w) kappa casein. The casein content of hybrid micelles or hybrid micelle compositions may comprise from 10-15% (w/w) alpha casein, 10-30% (w/w) beta casein and 55-80% (w/w) kappa casein. The casein content of hybrid micelles or hybrid micelle compositions may comprise from 15-25% (w/w) alpha casein, 10-30% (w/w) beta casein and 45-75% (w/w) kappa casein. The casein content of hybrid micelles or hybrid micelle compositions may comprise from 25-35% (w/w) alpha casein, 10-30% (w/w) beta casein and 35-65% (w/w) kappa casein. The casein content of hybrid micelles or hybrid micelle compositions may comprise from 35-45% (w/w) alpha casein, 10-30% (w/w) beta casein and 45-55% (w/w) kappa casein. The casein content of hybrid micelles or hybrid micelle compositions may comprise from 45-55% (w/w) alpha casein, 10-30% (w/w) beta casein and 15-45% (w/w) kappa casein. The casein content of hybrid micelles or hybrid micelle compositions may comprise from 55-65% (w/w) alpha casein, 10-30% (w/w) beta casein and 5-35% (w/w) kappa casein.

The casein content of hybrid micelles or hybrid micelle compositions may comprise from 1-10% (w/w) alpha casein, 30-50% (w/w) beta casein and 40-69% (w/w) kappa casein. The casein content of hybrid micelles or hybrid micelle compositions may comprise from 10-15% (w/w) alpha casein, 30-50% (w/w) beta casein and 35-60% (w/w) kappa casein. The casein content of hybrid micelles or hybrid micelle compositions may comprise from 15-25% (w/w) alpha casein, 30-50% (w/w) beta casein and 25-55% (w/w) kappa casein. The casein content of hybrid micelles or hybrid micelle compositions may comprise from 25-35% (w/w) alpha casein, 30-50% (w/w) beta casein and 15-45% (w/w) kappa casein. The casein content of hybrid micelles or hybrid micelle compositions may comprise from 35-45% (w/w) alpha casein, 30-50% (w/w) beta casein and 5-35% (w/w) kappa casein.

The casein content of hybrid micelles or hybrid micelle compositions may comprise from 1-10% (w/w) alpha casein, 50-70% (w/w) beta casein and 20-49% (w/w) kappa casein. The casein content of hybrid micelles or hybrid micelle compositions may comprise from 10-15% (w/w) alpha casein, 50-70% (w/w) beta casein and 15-40% (w/w) kappa casein. The casein content of hybrid micelles or hybrid micelle compositions may comprise from 15-25% (w/w) alpha casein, 50-70% (w/w) beta casein and 5-35% (w/w) kappa casein.

The casein content of hybrid micelles or hybrid micelle compositions may comprise from 1-9% (w/w) alpha casein, 70-90% (w/w) beta casein and 1-29% (w/w) kappa casein.

The casein content of hybrid micelles or hybrid micelle compositions may comprise from about 5% kappa and about 95% alpha casein proteins to about 50% kappa and about 50% alpha casein proteins wt/wt. The casein content of hybrid micelles or hybrid micelle compositions may comprise about 6% kappa and about 94% alpha, about 5% kappa and about 95% alpha about 7% kappa and about 93% alpha, about 10% kappa and about 90%, alpha, about 12% kappa and about 88% alpha, about 15% kappa and about 85% alpha, about 17% kappa and about 83% alpha, about 20% kappa and about 80% alpha, about 25% kappa and about 75% alpha, about 30% kappa and about 70% alpha casein proteins, about 35% kappa and about 65% alpha, about 40% kappa and about 60% alpha, about 45% kappa and about 55% alpha or about 50% kappa and about 50% alpha wt/wt.

The casein content of hybrid micelles or hybrid micelle compositions may comprise from about 5% kappa and about 95% beta casein proteins to about 50% kappa and about 50% beta casein proteins wt/wt. The casein content of hybrid micelles or hybrid micelle compositions may comprise about 6% kappa and about 94% beta, about 5% kappa and about 95% beta, about 7% kappa and about 93% beta, about 10% kappa and about 90% beta, about 12% kappa and about 88% beta, about 15% kappa and about 85% beta, about 17% kappa and about 83% beta, about 20% kappa and about 80% beta, about 25% kappa and about 75% beta, about 30% kappa and about 70% beta casein proteins, about 35% kappa and about 65% beta, about 40% kappa and about 60% beta, about 45% kappa and about 55% beta or about 50% kappa and about 50% beta wt/wt.

Alpha Casein (α casein): In some embodiments, hybrid micelles herein may comprise alpha casein proteins. The alpha casein in hybrid micelles may be alpha-S1-casein. The alpha casein in hybrid micelles may be alpha-S2-casein. The alpha casein in hybrid micelles may be a combination of alpha-S1 and alpha-S2 caseins. The alpha casein in hybrid micelles may comprise from 0% to 100% (w/w) of casein. In some instances, hybrid micelles or hybrid micelle compositions may be produced without any alpha casein. In some cases, the alpha casein comprises at least 10%, 20%, 30%, 40%, 50%, 60%, 70%, 80%, 90% or 95% of the casein in hybrid micelles. The alpha casein in hybrid micelles may comprise from 0% to 100% alpha-S1-casein, alpha-S2-casein or a combination thereof.

Alpha casein protein may be a mammalian alpha casein protein, in some cases, from a ruminant species. Alpha casein protein may be a bovine alpha casein protein. Alpha casein may be a caprine alpha casein protein. Alpha casein protein may be an ovine alpha casein protein. Alpha casein protein may be an equine alpha casein protein. Alpha casein protein may be a camel alpha casein protein.

In some cases, casein in hybrid micelles comprise of 50% alpha-S1-casein to 99% alpha-S1-casein w/w. In some cases, hybrid micelles comprise alpha casein protein and total alpha casein comprises 100% alpha-S1-casein w/w. In some cases, hybrid micelles comprise alpha casein protein and the total casein comprises at least 50% alpha-S1-casein. The alpha casein protein in hybrid micelles may comprise from 50% alpha-S1-casein to 70% alpha-S1-casein, 50% alpha-S1-casein to 90% alpha-S1-casein, 50% alpha-S1-casein to 100% alpha-S1-casein, 70% alpha-S1-casein to 90% alpha-S1-casein, 70% alpha-S1-casein to 100% alpha-S1-casein, or 90% alpha-S1-casein to 100% alpha-S1-casein. The alpha casein protein in hybrid micelles may comprise about 50% alpha-S1-casein, 70% alpha-S1-casein, 90% alpha-S1-casein, or 100% alpha-S1-casein.

In some embodiments, the alpha casein in the hybrid micelles is alpha-S2-casein. In some cases, alpha casein in hybrid micelles comprise of 50% alpha-S2-casein to 100% alpha-S2-casein. In some cases, hybrid micelles comprise alpha casein protein and the total casein comprises 100% alpha-S2-casein. In some cases, hybrid micelles comprise alpha casein protein comprising at least 50% alpha-S2-casein. The alpha casein protein in hybrid micelles may comprise from 50% alpha-S2-casein to 70% alpha-S2-casein, 50% alpha-S2-casein to 90% alpha-S2-casein, 50% alpha-S2-casein to 100% alpha-S2-casein, 70% alpha-S2-casein to 90% alpha-S2-casein, 70% alpha-S2-casein to 100% alpha-S2-casein, or 90% alpha-S2-casein to 100% alpha-S2-casein. The alpha casein protein in hybrid micelles may comprise 50% alpha-S2-casein, 70% alpha-S2-casein, 90% alpha-S2-casein, or 100% alpha-S2-casein.

In some embodiments, the alpha casein in hybrid micelles is a mixture of alpha-S1-casein and alpha-S2-casein. The alpha casein in such hybrid micelles may comprise, for example from 1% alpha-S2-casein to 99% alpha-S2-casein and from 99% alpha-S1-casein to 1% alpha-S1-casein, respectively. In some embodiments, the alpha casein in hybrid micelles is a mixture of alpha-S1-casein and alpha-S2-casein in ratio of 10:90, 20:80, 30:70, 40:60, 50:50, 70:30, 80:20, or 90:10. In some cases, the alpha casein protein in hybrid micelles does not include alpha-S2-casein. In some cases, the alpha casein protein in hybrid micelles does not include alpha-S1-casein.

In some embodiments, the alpha casein in the hybrid micelles is a mixture of a full-length alpha casein and a truncated form of alpha casein. Truncated forms of alpha casein may comprise one or more truncations described herein, for instance any one of SEQ ID NOs: 4-11 or 86. The alpha casein in such hybrid micelles may comprise a small amount of truncated alpha casein protein, for instance from about 0.5% truncated alpha casein to about 10% truncated alpha casein and from about 99.5% full-length alpha casein to 90% full-length alpha casein. In some cases, a mixture of a full-length alpha casein and a truncated form of alpha casein comprises about 0.5% to about 10% of a truncated form of alpha casein. In some cases, a mixture of a full-length alpha casein and a truncated form of alpha casein comprises at least about 0.5% of a truncated form of alpha casein. In some cases, a mixture of a full-length alpha casein and a truncated form of alpha casein comprises at most about 10% of a truncated form of alpha casein. In some cases, a mixture of a full-length alpha casein and a truncated form of alpha casein comprises about 0.5% to about 1%, about 0.5% to about 2%, about 0.5% to about 3%, about 0.5% to about 5%, about 0.5% to about 7%, about 0.5% to about 10%, about 1% to about 2%, about 1% to about 3%, about 1% to about 5%, about 1% to about 7%, about 1% to about 10%, about 2% to about 3%, about 2% to about 5%, about 2% to about 7%, about 2% to about 10%, about 3% to about 5%, about 3% to about 7%, about 3% to about 10%, about 5% to about 7%, about 5% to about 10%, or about 7% to about 10% of a truncated form of alpha casein. In some cases, a mixture of a full-length alpha casein and a truncated form of alpha casein comprises about 0.5%, about 1%, about 2%, about 3%, about 5%, about 7%, or about 10% of a truncated form of alpha casein.

In some cases, a mixture of a full-length alpha casein and a truncated form of alpha casein comprises a larger amount of truncated alpha casein, for instance from about 5% to about 30% of a truncated form of alpha casein. In some cases, a mixture of a full-length alpha casein and a truncated form of alpha casein comprises at least about 5% of a truncated form of alpha casein. In some cases, a mixture of a full-length alpha casein and a truncated form of alpha casein comprises at most about 30% of a truncated form of alpha casein. In some cases, a mixture of a full-length alpha casein and a truncated form of alpha casein comprises about 5% to about 10%, about 5% to about 20%, about 5% to about 30%, about 10% to about 20%, about 10% to about 30%, or about 20% to about 30% of a truncated form of alpha casein. In some cases, a mixture of a full-length alpha casein and a truncated form of alpha casein comprises about 5%, about 10%, about 20%, or about 30% of a truncated form of alpha casein. Alternatively, the amount of truncated alpha casein in a mixture of full-length and truncated alpha casein may be increased to produce different types of products.

The casein content of hybrid micelles or hybrid micelle compositions herein may comprise from 30% to 90%, or 50% to 95% alpha casein protein. In some cases, the casein content of hybrid micelles or hybrid micelle compositions may comprise at least 30% alpha casein protein. In some cases, the casein content of hybrid micelles or hybrid micelle compositions may comprise at least 50% alpha casein protein. In some cases, the casein content of hybrid micelles or hybrid micelle compositions may comprise at least 90% or at least 95% alpha casein protein. The casein content of hybrid micelles or hybrid micelle compositions may comprise from 30% to 35%, 30% to 40%, 30% to 50%, 30% to 55%, 30% to 70%, 30% to 75%, 30% to 80%, 30% to 85%, 30% to 90%, 35% to 40%, 35% to 50%, 35% to 55%, 35% to 70%, 35% to 75%, 35% to 80%, 35% to 85%, 35% to 90%, 40% to 50%, 40% to 55%, 40% to 70%, 40% to 75%, 40% to 80%, 40% to 85%, 40% to 90%, 50% to 55%, 50% to 70%, 50% to 75%, 50% to 80%, 50% to 85%, 50% to 90%, 55% to 70%, 55% to 75%, 55% to 80%, 55% to 85%, 55% to 90%, 70% to 75%, 70% to 80%, 70% to 85%, 70% to 90%, 75% to 80%, 75% to 85%, 75% to 90%, 80% to 85%, 80% to 90%, 85% to 90%, or 90 to 95% alpha casein protein. The casein content of hybrid micelles or hybrid micelle compositions may comprise 30%, 35%, 40%, 50%, 55%, 70%, 75%, 80%, 85%, 90% or 95% alpha casein protein. The casein content of hybrid micelles or hybrid micelle compositions may comprise at least 30%, at least 35%, at least 40%, at least 50%, at least 55%, at least 60%, at least 65%, at least 70%, at least 75%, at least 80%, at least 85%, or at least 90% alpha casein protein. The casein content of hybrid micelles or hybrid micelle compositions may comprise at most 40%, at most 50%, at most 55%, at most 60%, at most 65%, at most 70%, at most 75%, at most 80%, at most 85%, at most 90%, or at most 95% alpha casein protein.

Alpha casein protein may be a mammalian alpha casein protein, in some cases a ruminant species alpha casein protein. Alpha casein protein may be a bovine alpha casein protein, for instance, casein protein with at least 90%, at least 92%, at least 95%, at least 97%, at least 99% sequence identity to any one of SEQ ID NOs: 1-11, 18-20, 30-32 or 39-41. Alpha casein may be an ovine alpha casein protein, for instance, casein protein with at least sequence identity to any one of SEQ ID NOs: 12-14 or 33-35. Alpha casein protein may be a caprine alpha casein protein, for instance, casein protein with at least 90%, at least 92%, at least 95%, at least 97%, at least 99% sequence identity to any one of SEQ ID NOs: 15-17 or 36-38. Alpha casein protein may be an equine alpha casein protein, for instance, casein protein with at least 90% sequence identity to any one of SEQ ID NOs: 21-23 or 42-44. Alpha casein protein may be a camel alpha casein protein, for instance, casein protein with at least 90%, at least 92%, at least 95%, at least 97%, at least 99% sequence identity to any one of SEQ ID NOs: 24-26 or Alpha casein protein may be a human alpha casein protein, for instance, casein protein with at least 90%, at least 92%, at least 95%, at least 97%, at least 99% sequence identity to SEQ ID NOs: 27-29. Alpha casein protein may be a truncated alpha casein protein, for instance, casein protein with at least 90%, at least 92%, at least 95%, at least 97%, at least 99% sequence identity to SEQ ID NOs: 4-11 or 86.

Beta Casein (β casein): In some embodiments, hybrid micelles herein comprise beta casein protein. Hybrid micelles described herein may be generated to comprise less than 10% beta casein protein. The casein content of hybrid micelles herein may comprise less than 10%, less than 8%, less than 5%, less than 3%, less than 2%, less than 1% or less than 0.5% beta casein protein. In some embodiments, the hybrid micelles described herein do not include any beta casein protein.

The casein content of hybrid micelles or hybrid micelle compositions herein may comprise from 30% to 90%, or 50% to 95% beta casein protein. In some cases, the casein content of hybrid micelles or hybrid micelle compositions may comprise at least 30% beta casein protein. In some cases, the casein content of hybrid micelles or hybrid micelle compositions may comprise at least 50% beta casein protein. In some cases, the casein content of hybrid micelles or hybrid micelle compositions may comprise at least 90% or at least 95% beta casein protein. The casein content of hybrid micelles or hybrid micelle compositions may comprise from 30% to 35%, 30% to 40%, 30% to 50%, 30% to 55%, 30% to 70%, 30% to 75%, 30% to 80%, 30% to 85%, 30% to 90%, 35% to 40%, 35% to 50%, 35% to 55%, 35% to 70%, 35% to 75%, 35% to 80%, 35% to 85%, 35% to 90%, 40% to 50%, 40% to 55%, 40% to 70%, 40% to 75%, 40% to 80%, 40% to 85%, 40% to 90%, 50% to 55%, 50% to 70%, 50% to 75%, 50% to 80%, 50% to 85%, 50% to 90%, 55% to 70%, 55% to 75%, 55% to 80%, 55% to 85%, 55% to 90%, 70% to 75%, 70% to 80%, 70% to 85%, 70% to 90%, 75% to 80%, 75% to 85%, 75% to 90%, 80% to 85%, 80% to 90%, 85% to 90%, or 90 to 95% beta casein protein. The casein content of hybrid micelles or hybrid micelle compositions may comprise 30%, 35%, 40%, 50%, 55%, 70%, 75%, 80%, 85%, 90% or 95% beta casein protein. The casein content of hybrid micelles or hybrid micelle compositions may comprise at least 30%, at least 35%, at least 40%, at least 50%, at least 55%, at least 60%, at least 65%, at least 70%, at least 75%, at least 80%, at least 85%, or at least 90% beta casein protein. The casein content of hybrid micelles or hybrid micelle compositions may comprise at most 40%, at most 50%, at most 55%, at most 60%, at most 65%, at most 70%, at most 75%, at most 80%, at most 85%, at most 90%, or at most 95% beta casein protein.

Beta casein protein may be a full-length beta casein protein, a truncated beta casein such as gamma casein protein or another truncation of the beta casein protein. In some cases, the truncation of the beta casein protein may be one other than the natural breakpoint of beta (e.g., different from the breakpoint creating gamma casein).

Beta casein protein may be a mammalian beta casein protein, in some cases a ruminant species beta casein protein. Beta casein protein may be a bovine beta casein protein. Beta casein may be a caprine beta casein protein. Beta casein protein may be an ovine beta casein protein. Beta casein protein may be an equine beta casein protein. Beta casein protein may be a camel beta casein protein.

Beta casein protein may be a mammalian beta casein protein, in some cases a ruminant species beta casein protein. Beta casein protein may be a bovine beta casein protein, for instance, casein protein with at least 90%, at least 92%, at least 95%, at least 97%, at least 99% sequence identity to any one of SEQ ID NO: 71-73 or 78-79. Beta casein may be an ovine beta casein protein, for instance, casein protein with at least 90%, at least 92%, at least 95%, at least 97%, at least 99% sequence identity to any one of SEQ ID NOs: 74-75. Beta casein protein may be a caprine beta casein protein, for instance, casein protein with at least 90%, at least 92%, at least 95%, at least 97%, at least 99% sequence identity to any one of SEQ ID NOs: 76-77. Beta casein protein may be an equine beta casein protein, for instance, casein protein with at least 90%, at least 92%, at least 95%, at least 97%, at least 99% sequence identity to any one of SEQ ID NOs: 80-81. Beta casein protein may be a camel beta casein protein, for instance, casein protein with at least 90%, at least 92%, at least 95%, at least 97%, at least 99% sequence identity to any one of SEQ ID NOs: 82-83. Beta casein protein may be a human beta casein protein, for instance, casein protein with at least 90%, at least 92%, at least 95%, at least 97%, at least 99% sequence identity to any one of SEQ ID NOs: 84-85.

Kappa Casein (κ casein): In some embodiments, hybrid micelles herein may comprise kappa casein proteins. The casein content of hybrid micelles may comprise from 0% to 100% kappa casein protein. The casein content of hybrid micelles may comprise at least 1% kappa casein protein. The casein content of hybrid micelles may comprise 100% or at most 50% or at most 30% kappa casein protein. The casein content of hybrid micelles may comprise from 1% to 5%, 1% to 7%, 1% to 10%, 1% to 12%, 1% to 15%, 1% to 18%, 1% to 20%, 1% to 25%, 1% to 30%, 5% to 7%, 5% to 10%, 5% to 12%, 5% to 15%, 5% to 18%, 5% to 20%, 5% to 25%, 5% to 30%, 7% to 10%, 7% to 12%, 7% to 15%, 7% to 18%, 7% to 20%, 7% to 25%, 7% to 30%, 10% to 12%, 10% to 15%, 10% to 18%, 10% to 20%, 10% to 25%, 10% to 30%, 12% to 15%, 12% to 18%, 12% to 20%, 12% to 25%, 12% to 30%, 15% to 18%, 15% to 20%, 15% to 25%, 15% to 30%, 18% to 20%, 18% to 25%, 18% to 30%, 20% to 25%, 20% to 30%, 25% to 30%, 30% to 35%, 35% to 40%, 40 to 45%, or 45% to 50% kappa casein protein. The casein content of hybrid micelles may comprise 1%, 5%, 7%, 10%, 12%, 15%, 18%, 20%, 25%, 30%, 35%, 40%, 45% or 50%, 60%, 70%, 80%, 90%, or 99% kappa casein protein. The casein content of hybrid micelles may comprise at least 1%, at least 5%, at least 7%, at least 10%, at least 12%, at least 15%, at least 18%, at least 20%, at least 25%, at least 30%, at least 35%, at least 40% or at least 45% kappa casein protein. The casein content of hybrid micelles may comprise at most 5%, at most 7%, at most 10%, at most 12%, at most 15%, at most 18%, at most 20%, at most 25%, at most 30%, at most 35%, at most 40%, at most 45% or at most 50% kappa casein protein. In some instances, a hybrid micelle may be produced using only kappa casein. Alternatively, in some cases, a hybrid micelle may be produced without any kappa casein.

Kappa casein protein may be a mammalian kappa casein protein, in some cases a ruminant species kappa casein protein. Kappa casein protein may be a bovine kappa casein protein, for instance, casein protein with at least 90%, at least 92%, at least 95%, at least 97%, at least 99% sequence identity to any one of SEQ ID NOs: 48-52 or 59-61. Kappa casein may be an ovine kappa casein protein, for instance, casein protein with at least 90%, at least 92%, at least 95%, at least 97%, at least 99% sequence identity to any one of SEQ ID NO: 53-55. Kappa casein protein may be a caprine kappa casein protein, for instance, casein protein with at least 90%, at least 92%, at least 95%, at least 97%, at least 99% sequence identity to any one of SEQ ID NOs: 56-58. Kappa casein protein may be an equine kappa casein protein, for instance, casein protein with at least 90%, at least 92%, at least 95%, at least 97%, at least 99% sequence identity to any one of SEQ ID NOs: 62-64. Kappa casein protein may be a camel kappa casein protein, for instance, casein protein with at least 90%, at least 92%, at least 95%, at least 97%, at least 99% sequence identity to any one of SEQ ID NOs: 65-67. Kappa casein protein may be a human kappa casein protein, for instance, casein protein with at least 90%, at least 92%, at least 95%, at least 97%, at least 99% sequence identity to any one of SEQ ID NOs: 68-70. Kappa casein protein may be a truncated kappa casein protein, for instance, casein protein with at least 90%, at least 92%, at least 95%, at least 97%, at least 99% sequence identity to SEQ ID NOs: 51-52.

In some embodiments, the kappa casein in the hybrid micelles is a mixture of a full-length kappa casein and a truncated form of kappa casein. The kappa casein in such hybrid micelles may comprise, for instance from about 0.5% truncated kappa casein to about 30% truncated kappa casein and from about 99.5% full-length kappa casein to 70% full-length kappa casein. In some cases, a mixture of a full-length kappa casein and a truncated form of kappa casein comprises about 0.5% to about 30% of a truncated form of kappa casein. In some cases, a mixture of a full-length kappa casein and a truncated form of kappa casein comprises at least about 0.5% of a truncated form of kappa casein. In some cases, a mixture of a full-length kappa casein and a truncated form of kappa casein comprises at most about 30% of a truncated form of kappa casein. In some cases, a mixture of a full-length kappa casein and a truncated form of kappa casein comprises about 0.5% to about 1%, about 0.5% to about 5%, about 0.5% to about 10%, about 0.5% to about 20%, about 0.5% to about 30%, about 1% to about 5%, about 1% to about 10%, about 1% to about 20%, about 1% to about 30%, about 5% to about 10%, about 5% to about 20%, about 5% to about 30%, about 10% to about 20%, about 10% to about 30%, or about 20% to about 30% of a truncated form of kappa casein. In some cases, a mixture of a full-length kappa casein and a truncated form of kappa casein comprises about 0.5%, about 1%, about 5%, about 10%, about 20%, or about 30% of a truncated form of kappa casein.

The ratio of alpha casein protein to kappa casein protein in hybrid micelles may be from about 1:1 to about 15:1. The ratio of alpha casein protein to kappa casein protein in hybrid micelles may be 1:1, 2:1 to 4:1, 2:1 to 6:1, 2:1 to 8:1, 2:1 to 10:1, 2:1 to 12:1, 2:1 to 14:1, 2:1 to 15:1, 4:1 to 6:1, 4:1 to 8:1, 4:1 to 10:1, 4:1 to 12:1, 4:1 to 14:1, 4:1 to 15:1, 6:1 to 8:1, 6:1 to 6:1 to 12:1, 6:1 to 14:1, 6:1 to 15:1, 8:1 to 10:1, 8:1 to 12:1, 8:1 to 14:1, 8:1 to 15:1, 10:1 to 12:1, 10:1 to 14:1, 10:1 to 15:1, 12:1 to 14:1, 12:1 to 15:1, or 14:1 to 15:1. The ratio of alpha casein protein to kappa casein protein in hybrid micelles may be about 1:1, 2:1, 3:1, 4:1, 5:1, 6:1, 7:1, 8:1, 9:1, 10:1, 11:1, 12:1, 13:1, 14:1 or 15:1. In some aspects, the alpha casein in such hybrid micelles may be solely alpha-S1-casein. In some aspects, the alpha casein in such hybrid micelles may be solely alpha-S2-casein.

The ratio of beta casein protein to kappa casein protein in hybrid micelles may be from about 1:1 to about 15:1. The ratio of beta casein protein to kappa casein protein in hybrid micelles may be 1:1, 2:1 to 4:1, 2:1 to 6:1, 2:1 to 8:1, 2:1 to 10:1, 2:1 to 12:1, 2:1 to 14:1, 2:1 to 15:1, 4:1 to 6:1, 4:1 to 8:1, 4:1 to 10:1, 4:1 to 12:1, 4:1 to 14:1, 4:1 to 15:1, 6:1 to 8:1, 6:1 to 10:1, 6:1 to 12:1, 6:1 to 14:1, 6:1 to 15:1, 8:1 to 10:1, 8:1 to 12:1, 8:1 to 14:1, 8:1 to 15:1, 10:1 to 12:1, 10:1 to 14:1, 10:1 to 15:1, 12:1 to 14:1, 12:1 to 15:1, or 14:1 to 15:1. The ratio of beta casein protein to kappa casein protein in hybrid micelles may be about 1:1, 2:1, 3:1, 4:1, 5:1, 6:1, 7:1, 8:1, 9:1, 10:1, 11:1, 12:1, 13:1, 14:1 or 15:1.

In some embodiments, hybrid micelles comprise alpha and kappa casein proteins and do not include beta casein, and additionally the alpha casein, kappa casein or both alpha and kappa casein lack post-translational modification(s). For example, hybrid micelles may comprise alpha casein lacking or substantially reduced in phosphorylation (as compared to alpha casein from animal-derived milk) and kappa casein, or comprises alpha casein lacking or substantially reduced in phosphorylation (as compared to alpha casein from animal-derived milk) and kappa casein that lacks or is substantially reduced in glycosylation or phosphorylation or both glycosylation and phosphorylation (as compared to kappa casein from animal-derived milk). In some cases, hybrid micelles comprise alpha casein and comprise kappa casein where the kappa casein is lacking or substantially reduced in glycosylation or phosphorylation or both glycosylation and phosphorylation (as compared to kappa casein from animal-derived milk). In some cases, hybrid micelles comprise alpha casein, kappa casein or both produced recombinantly in a bacterial host cell and that lack or are substantially reduced in one or more PTMs.

In some embodiments, hybrid micelles herein (and products made therefrom) do not include any dairy-obtained proteins other than alpha and kappa casein proteins. In some cases, hybrid micelles herein (and products made therefrom) do not include any whey proteins. In some embodiments, hybrid micelles herein (and products made therefrom) do not include any animal-obtained dairy proteins.

### Salts

The hybrid micelles described herein may be present in a colloid form such as in a liquid colloid and may comprise alpha and kappa casein, and in some cases beta casein proteins as described elsewhere herein. In some embodiments, the colloid includes alpha casein and kappa casein, but does not include beta casein. Micelle formation, such as the hybrid micelles described elsewhere herein, in liquid colloid herein may comprise addition of various salts to a solution comprising a casein mixture or micelle composition. Salts that may be added to a casein mixture or hybrid micelle composition may include calcium, phosphorous, citrate, potassium, sodium and/or chloride salts. In some cases, salt is comprised within the hybrid micelles. In some cases, salt is comprised in the liquid colloid such that a proportion of salt is comprised in the hybrid micelles and another portion of salt is in solution (e.g., “outside” the micelles).

Liquid colloid containing hybrid casein micelles may comprise a calcium salt. The calcium salt may be selected from calcium chloride, calcium carbonate, calcium citrate, calcium glubionate, calcium lactate, calcium gluconate, calcium acetate, equivalents thereof and/or combinations thereof. The concentration of a calcium salt in liquid colloid may be from about 10 mM to about 55 mM. The concentration of a calcium salt in liquid colloid may be at least 10 mM. The concentration of a calcium salt in liquid colloid may be at most 50 mM. In some embodiments, the concentration of a calcium salt in liquid colloid may be 28 mM or no more than 28 mM or may be 55 mM or no more than 55 mM. The concentration of a calcium salt in liquid colloid may be about 10 mM to 15 mM, 10 mM to 20 mM, 10 mM to 25 mM, 10 mM to 30 mM, 10 mM to 35 mM, 10 mM to 40 mM, 10 mM to 45 mM, 10 mM to 50 mM, 10 mM to 55 mM, 15 mM to 20 mM, 15 mM to 25 mM, 15 mM to 30 mM, 15 mM to 35 mM, 15 mM to 40 mM, 15 mM to 45 mM, 15 mM to 50 mM, 15 mM to 55 mM, 20 mM to 25 mM, 20 mM to 30 mM, 20 mM to 35 mM, 20 mM to 40 mM, 20 mM to 45 mM, 20 mM to 50 mM, 20 mM to 55 mM, 25 mM to 30 mM, 25 mM to 35 mM, 25 mM to 40 mM, 25 mM to 45 mM, 25 mM to 50 mM, 25 mM to 55 mM, 30 mM to 35 mM, 30 mM to 40 mM, 30 mM to 45 mM, 30 mM to 50 mM, 30 mM to 55 mM, 35 mM to 40 mM, 35 mM to 45 mM, 35 mM to 50 mM, 35 mM to 55 mM, 40 mM to 45 mM, 40 mM to 50 mM, 40 mM to 55 mM, 45 mM to 50 mM, 45 mM to 55 mM, or 50 mM to 55 mM. The concentration of a calcium salt in liquid colloid may be 10 mM, 15 mM, 20 mM, 25 mM, 30 mM, 35 mM, 40 mM, 45 mM, 50 mM, or 55 mM. The concentration of a calcium salt in liquid colloid may be at least 10 mM, at least 15 mM, at least 20 mM, at least 25 mM, at least 30 mM, at least 35 mM, at least 40 mM, at least 45 mM or at least 50 mM. The concentration of a calcium salt in liquid colloid may be at most 15 mM, at most 20 mM, at most 25 mM, at most 30 mM, at most 35 mM, at most 40 mM, at most 45 mM, at most 50 mM or at most 55 mM.

Liquid colloid containing casein micelles may comprise a phosphate salt. The phosphate salt may be selected from orthophosphates such as monosodium (dihydrogen) phosphate, disodium phosphate, trisodium phosphate, monopotassium (dihydrogen) phosphate, dipotassium phosphate, tripotassium phosphate; pyrophosphates such as disodium or dipotassium pyrophosphate, trisodium or tripotassium pyrophosphate, tetrasodium or tetrapotassium pyrophosphate; polyphosphates such as pent sodium or potassium tripolyphosphate, sodium or potassium tetrapolyphosphate, sodium or potassium hexametaphosphate. The concentration of a phosphate salt in liquid colloid may be from about 8 mM to about 45 mM. The concentration of a phosphate salt in liquid colloid may be at least 8 mM. The concentration of a phosphate salt in liquid colloid may be at most 25 mM or at most mM or at most 40 mM or at most 45 mM. The concentration of a phosphate salt in liquid colloid may be about 8 mM to 10 mM, 8 mM to 15 mM, 8 mM to 20 mM, 8 mM to 25 mM, 8 mM to 30 mM, 8 mM to 35 mM, 8 mM to 40 mM, 8 mM to 45 mM, 10 mM to 15 mM, 10 mM to 20 mM, 10 mM to 25 mM, 10 mM to 30 mM, 10 mM to 35 mM, 10 mM to 40 mM, 10 mM to 45 mM, 15 mM to 20 mM, 15 mM to 25 mM, 15 mM to 30 mM, 15 mM to 35 mM, 15 mM to 40 mM, 15 mM to 45 mM, 20 mM to 25 mM, 20 mM to 30 mM, 20 mM to 35 mM, 20 mM to 40 mM, 20 mM to 45 mM, 25 mM to 30 mM, 25 mM to 35 mM, 25 mM to 40 mM, 25 mM to 45 mM, 30 mM to 35 mM, 30 mM to 40 mM, 30 mM to 45 mM, 35 mM to 40 mM, 35 mM to 45 mM, or 40 mM to 45 mM. The concentration of a phosphate salt in liquid colloid may be about 8 mM, 10 mM, 15 mM, 20 mM, 25 mM, 30 mM, 35 mM, 40 mM, or 45 mM. The concentration of a phosphate salt in liquid colloid may be at least 8 mM, at least 10 mM, at least 15 mM, at least 20 mM, at least 25 mM, at least 30 mM, at least 35 mM or at least 40 mM. The concentration of a phosphate salt in liquid colloid may be at most 10 mM, at most 15 mM, at most 20 mM, at most 25 mM, at most 30 mM, at most 35 mM, at most 40 mM or at most 45 mM.

Liquid colloid containing casein micelles may comprise a citrate salt. The citrate salt may be selected from calcium citrate, potassium citrate, sodium citrate, trisodium citrate, tripotassium citrate or equivalents thereof. The concentration of a citrate salt in liquid colloid may be from about 2 mM to about 20 mM. The concentration of a citrate salt in liquid colloid may be at least 2 mM. The concentration of a citrate salt in liquid colloid may be at most 15 mM or at most 20 mM. The concentration of a citrate salt in liquid colloid may be about 2 mM to 4 mM, 2 mM to 6 mM, 2 mM to 8 mM, 2 mM to 10 mM, 2 mM to 12 mM, 2 mM to 14 mM, 2 mM to 16 mM, 2 mM to 18 mM, 2 mM to 20 mM, 4 mM to 6 mM, 4 mM to 8 mM, 4 mM to 10 mM, 4 mM to 12 mM, 4 mM to 14 mM, 4 mM to 16 mM, 4 mM to 18 mM, 4 mM to 20 mM, 6 mM to 8 mM, 6 mM to 10 mM, 6 mM to 12 mM, 6 mM to 14 mM, 6 mM to 16 mM, 6 mM to 18 mM, 6 mM to 20 mM, 8 mM to 10 mM, 8 mM to 12 mM, 8 mM to 14 mM, 8 mM to 16 mM, 8 mM to 18 mM, 8 mM to 20 mM, 10 mM to 12 mM, 10 mM to 14 mM, 10 mM to 16 mM, 10 mM to 18 mM, 10 mM to 20 mM, 12 mM to 14 mM, 12 mM to 16 mM, 12 mM to 18 mM, 12 mM to 20 mM, 14 mM to 16 mM, 14 mM to 18 mM, 14 mM to 20 mM, 16 mM to 18 mM, 16 mM to 20 mM, or 18 mM to 20 mM. The concentration of a citrate salt in liquid colloid may be 2 mM, 4 mM, 6 mM, 8 mM, 10 mM, 12 mM, 14 mM, 16 mM, 18 mM, or 20 mM. The concentration of a citrate salt in liquid colloid may be at least 2 mM, at least 4 mM, at least 6 mM, at least 8 mM, at least 10 mM, at least 12 mM, at least 14 mM, at least 16 mM or at least 18 mM. The concentration of a citrate salt in liquid colloid may be at most 4 mM, at most 6 mM, at most 8 mM, at most 10 mM, at most 12 mM, at most 14 mM, at most 16 mM, at most 18 mM, or at most 20 mM.

Liquid colloid containing casein micelles may comprise a combination of salts. In some embodiments, the liquid colloid comprises calcium, phosphate and citrate salts. In some cases, a ratio of calcium, phosphate and citrate salt in liquid colloid may be from 3:2:1 to about 6:4:1. A ratio of calcium, phosphate and citrate salt in liquid colloid may be about 3:1:1, 3:2:1, 3:3:1, 4:2:1, 4:3:1, 4:4:1, 5:2:1, 5:2:2, 5:3:1, 5:4:1, 5:5:1, 5:3:2, 5:4:2, 6:1:1, 6:2:1, 6:3:1 or 6:4:1.

Micelle formation in liquid colloid may require solubilization of casein proteins in a solvent such as water. Salts may be added after the solubilization of casein proteins in a solvent. Alternatively, salts and casein proteins may be added to the solution simultaneously. Salts may be added more than once during micelle formation. For instance, calcium salts, phosphate salts and citrate salts may be added at regular intervals or in a continuous titration process and mixed in a solution comprising casein proteins until a micellar liquid colloid of desired quality is generated. In one example, salts may be added at regular interval until the colloid reaches a desired concentration. Different salts may be added at different times during the micelle formation process. For instance, calcium salts may be added before the addition of phosphate and citrate salts, or citrate salts may be added before the addition of calcium and phosphate salts, or phosphate salts might be added before the addition of calcium and citrate salts.

Additional components may be added to liquid colloid such that the liquid colloid is then milk-like and used for curd and/or cheese or yogurt formation. In some embodiments, fat is added to liquid colloid. In some cases, fats may be essentially free of animal-derived fats. Fats used herein may include plant-based fats such as canola oil, sunflower oil, coconut oil or combinations thereof. The concentration of fats may be about 0% to about 5% in the liquid colloid. The concentration of fats may be at least 0.5% or about 1%. The concentration of fats may be at most 5%. The concentration of fats may be about 0%, 0.1%, 0.5%, 1%, 2%, 3%, 4% or 5%. The concentration of fats may be from 0 to 0.5%, 0.5% to 1%, 1% to 3%, 1% to 4%, or 1% to 5%. The concentration of fats may be at most 2%, at most 3%, at most 4%, or at most 5%.

Liquid colloid as described herein may further comprise sugars. Sugars used herein may include plant-based oligosaccharides and/or monosaccharides/disaccharides. Examples of sugars include sucrose, glucose, fructose, galactose, lactose, maltose, mannose, allulose, tagatose, xylose, and arabinose.

Liquid colloid with additional components may be generated by mixing different components at a temperature from 20° C. to 90° C. For instance, liquid colloid with one or more recombinant proteins (such as a combination of alpha and kappa casein) may be mixed with fats and/or sugars at a temperature of about 20° C., 35° C., 30° C., 32° C., 35° C., 37° C., 40° C., 42° C., 50° C., 55° C., 60° C., 70° C., 75° C., 80° C., 85° C., 90° C.

### IV. Consumable Compositions

Micelles, such as the hybrid micellar compositions or liquid colloids described herein may be used to produce a variety of consumable compositions. Hybrid micelles with different types of caseins may lead to the production of different consumable compositions with specific properties. For instance, a soft and spreadable cheese like a goat-like cheese may be made using hybrid micelles comprising a bovine alpha casein protein combined with a goat kappa casein protein. In another instance, a crumbly cheese such as a feta cheese may be made with a different combination of casein proteins in a hybrid micelle. In some cases, different concentrations of casein proteins may provide specific properties to a consumable composition. In one example, the yield or melt or stretch of a cheese may be dependent on the ratio of alpha to kappa casein proteins.

Micelles, such as the hybrid micellar compositions or liquid colloids described herein may be dried to produce a micellar casein containing protein powder. Methods for drying micelles, micellar compositions and/or liquid colloids may include, but are not limited to, spray drying, roller drying, fluid bed drying, freeze drying, drying with ethanol, and evaporating.

Casein containing protein powder may be generated by subjecting the hybrid micelles within a liquid colloid to salt precipitation. Casein containing protein powder may be generated by subjecting the hybrid micelles within a liquid colloid to acid precipitation. Herein described casein containing protein powder may be used as an ingredient in a consumable food product. For instance, the casein containing protein powder may be used as an ingredient in the production of milk, milkshakes, beverages, snacks, creamers, condensed milk, cream, ice-cream, yogurt, mozzarella cheese analogue, curd, cheese and/or any other dairy-obtained products.

Hybrid micelles such as micelles of alpha and kappa casein or as micelles described elsewhere herein, may be present in a liquid colloid, where a substantial portion of the micelles remain in suspension in the liquid. In some embodiments, the hybrid micelles are present in a solid form, such as by drying, freezing or spray-drying the liquid colloid.

In some embodiments, the liquid colloid is treated to form a coagulated colloid. In some cases, the treatment is a reduction of pH of the liquid colloid such as by adding acid or acidifying with a microorganism, to generate coagulated colloid.

Fats may be added to liquid colloid for the generation of a coagulated colloid or curds such that in a final cheese product the concentration of fat is between about 0% to about 50%, typically more than 0%. For example, the concentration of fat in the cheese product made from liquid colloid is about 5%, 10%, 15%, 20%, 25%, 30%, 35%, 40%, 45% or 50%. The concentration of fat in the cheese product made from liquid colloid may be 1% to 50%. The concentration of fat in the cheese product made from liquid colloid may be at least 1%. The concentration of fat in the cheese product made from liquid colloid may be at most 50%. The concentration of fat in the cheese product made from liquid colloid may be about 1% to 5%, 1% to 10%, 1% to 15%, 1% to 20%, 1% to 25%, 1% to 30%, 1% to 35%, 1% to 40%, 1% to 45%, 1% to 50%, 5% to 10%, 5% to 15%, 5% to 20%, 5% to 25%, 5% to 30%, 5% to 35%, 5% to 40%, 5% to 45%, 5% to 50%, 10% to 15%, 10% to 20%, 10% to 25%, 10% to 30%, 10% to 35%, 10% to 40%, 10% to 45%, 10% to 50%, 15% to 20%, 15% to 25%, 15% to 30%, 15% to 35%, 15% to 40%, 15% to 45%, 15% to 50%, 20% to 25%, 20% to 30%, 20% to 35%, 20% to 40%, 20% to 45%, 20% to 50%, 25% to 30%, 25% to 35%, 25% to 40%, 25% to 45%, 25% to 50%, 30% to 35%, 30% to 40%, 30% to 45%, 30% to 50%, 35% to 40%, 35% to 45%, 35% to 50%, 40% to 45%, 40% to 50%, or 45% to 50%. The concentration of fat in the cheese product made from liquid colloid may be 1%, 5%, 10%, 15%, 20%, 25%, 30%, 35%, 40%, 45%, or 50%. The concentration of fat in the cheese product made from liquid colloid may be at least 1%, at least 5%, at least 10%, at least 15%, at least 20%, at least 25%, at least 30%, at least 35%, at least 40%, at least 45%, or at least 50%. The concentration of fat in the cheese/yogurt product made from liquid colloid may be at most 1%, at most 5%, at most 10%, at most 15%, at most 20%, at most 25%, at most 30%, at most 35%, at most 40% or at most 45%.

Fats may be emulsified into liquid colloid (e.g., comprising hybrid micelles formed with alpha, beta and kappa casein and salt) using sonication or high-pressure homogenization process. An emulsifier such as soy lecithin or xanthan gum may be used to secure a stable emulsion.

Coagulated colloid may be generated at a final pH of about 4 to about 6. Coagulated colloid may be generated at a pH of about 4 to about 6. Coagulated colloid may be generated at a final pH of at least 4. Coagulated colloid may be generated at a final pH of at most 6. Coagulated colloid may be generated at a final pH of 4 to 4.5, 4 to 5, 4 to 5.1, 4 to 5.2, 4 to to 6, 4.5 to 5, 4.5 to 5.1, 4.5 to 5.2, 4.5 to 5.5, 4.5 to 6, 5 to 5.1, 5 to 5.2, 5 to 5.5, 5 to 6, to 5.2, 5.1 to 5.5, 5.1 to 6, 5.2 to 5.5, 5.2 to 6, or 5.5 to 6. Coagulated colloid may be generated at a final pH of about 4, about 4.5, about 5, about 5.1, about 5.2, about 5.5, or about 6. Coagulated colloid may be generated at a final pH of at least 4, at least 4.5, at least 5, at least at least 5.2 or at least 5.5. Coagulated colloid may be generated at a final pH of at most 4.5, at most 5, at most 5.1, at most 5.2, at most 5.5, or at most 6. Treatments for reducing pH of liquid colloid and achieving a final pH or final pH range described herein may include the addition of an acid such as citric acid, lactic acid, or vinegar (acetic acid). Treatments for reducing pH of liquid colloid and achieving a final pH or final pH range described herein may include the addition of an acidifying microorganism such as lactic acid bacteria. Exemplary acidifying microorganisms include Lactococci, Streptococci, Lactobacilli and mixtures thereof. In some cases, both acid and an acidifying microorganism are added to the liquid colloid to create a coagulated colloid. In some cases, aging and ripening microorganisms (such as bacteria or fungi) are also added in this step.

In some cases, following acidification, a renneting agent may be added to form a renneted curd (coagulated curd matrix), which may then be used to make cheese. Hybrid micelles in a liquid colloid, such as milk and also the liquid colloid described herein, are stable and repel each other in colloidal suspension. In presence of renneting agents or milk-clotting enzymes, and when acidified, hybrid micelles are destabilized and attract each other, and thus coagulate. In presence of renneting agents or milk-clotting enzymes, cross-linked coagulated curd matrix is formed. Renneting agents used for curd formation may include chymosin, pepsin A, mucorpepsin, endothiapepsin or equivalents thereof. Renneting agents may be derived from plants, dairy products or recombinantly.

In some embodiments, renneted curd is further treated to create a cheese or cheese-like product. In some cases, such as a mozzarella product, the renneted curd may be heated and stretched. In other embodiments, the renneted curd is aged or matured, such as for brie, camembert, feta, halloumi, gouda, edam, cheddar, manchego, swiss, colby, muenster, blue cheese or parmesan type cheese or cheese-like product.

In some embodiments, coagulated colloid or renneted curd may be treated with hot water for the formation of cheese, such as for mozzarella-type cheese. Hot water treatment may be performed at a temperature of about 50° C. to about 90° C. Hot water treatment may be performed at a temperature of at least 55° C. Hot water treatment may be performed at a temperature of at most 75° C. Hot water treatment may be performed at a temperature of about to 55° C., 55° C. to 60° C., 55° C. to 65° C., 55° C. to 70° C., 55° C. to 75° C., 60° C. to 65° C., 60° C. to 70° C., 60° C. to 75° C., 65° C. to 70° C., 65° C. to 75° C., 70° C. to 75° C., 75° C. to 80° C., 80° C. to or 85° C. to 90° C. Hot water treatment may be performed at a temperature of about 50° C., about 55° C., about 60° C., about 65° C., about 70° C., about 75° C., about 80° C., about 85° C. or about Hot water treatment may be performed at a temperature of at least 50° C., at least 55° C., at least 60° C., at least 65° C., at least 70° C., at least 75° C., at least 80° C., or at least 85° C. Hot water treatment may be performed at a temperature of at most 55° C., at most 60° C., at most at most 70° C., at most 75° C., at most 80° C., at most 85° C. or at most 90° C. In some cases, after hot water treatment, the product is stretched into a cheese. In some cases, the cheese is a mozzarella-like cheese.

Cheese compositions formed using the methods described herein may not comprise any components obtained or isolated from animals. Cheese compositions formed using the methods described herein may not comprise any animal-obtained dairy-based components, such as animal-obtained dairy proteins. Cheese compositions formed using the methods described herein may not comprise any whey proteins. Cheese compositions formed using the methods described herein may include combinations of caseins that are not found in nature, for example where one or more of the alpha, beta, kappa caseins are from different species, and/or where one or more truncated caseins are incorporated into micelles prior to cheese formation. Cheese compositions formed using the methods described herein may not comprise any beta casein protein. Cheese compositions described herein may be pasta-filata like cheese such as mozzarella cheese. Soft cheeses such as paneer, cream cheese or cottage cheese may also be formed using the methods described herein. Other types of cheese such as aged and ripened cheeses may also be formed using the methods described herein, such as brie, camembert, feta, halloumi, gouda, edam, cheddar, manchego, swiss, colby, muenster, blue cheese and parmesan.

The texture of a cheese made by methods described herein may be comparable to the texture of a similar type of cheese made using animal-obtained dairy proteins, such as cheese made from animal milk. Texture of a cheese made using a hybrid micelle composition may be comparable to the texture of a similar type of cheese made using animal-obtained dairy proteins from any of the species whose caseins were used in the formation of the micelle. Texture of a cheese may be tested using a trained panel of human subjects or machines such as a texture analyzer.

The taste of a cheese made by methods described herein may be comparable to a similar type of cheese made using animal-obtained dairy proteins. Taste of a cheese made using a hybrid micelle composition may be comparable to the taste of a similar type of cheese made using animal-obtained dairy proteins from any of the species whose caseins were used in the formation of the micelle. Taste of a cheese may be tested using a trained panel of human subjects.

Cheese compositions described herein may have a browning ability which is comparable to a similar type of cheese made using animal-obtained dairy proteins. Cheese compositions described herein may have a melting ability which is comparable to a similar type of cheese made using animal-obtained dairy proteins.

In some embodiments, the liquid colloid may be used for yogurt formation. In some cases, for yogurt production, the liquid colloid may be heat treated. The heat treatment may include treating the liquid colloid at a temperature of about 75° C., 80° C., 85° C., 87° C., 90° C., 92° C., 95° C., or 100° C. The heat treatment may be followed with a cooling step of the liquid colloid.

In some cases, for instance, in yogurt or cheese production, a bacterial culture may be used as a starter culture. Starter bacterial cultures used for yogurt or cheese production may be any bacterial cultures known in the art. For instance, bacteria known for yogurt or cheese generation such as Lactobacillus delbrueckii subsp. bulgaricus, Streptococcus thermophilus, other lactobacilli and bifidobacteria sp. bacteria may be cultured and added to the liquid colloid comprising the one or more recombinant proteins. In some cases, oligosaccharides (monosaccharides/disaccharides/oligosaccharides) may be added along with a starter culture. The bacterial starter culture may be used for the acidification of the liquid colloid. Acidification of a liquid colloid may be continued until a desired consistency of the colloid is achieved. For instance, bacterial acidification may be continued until a desired consistency is reached for the liquid colloid. Bacterial acidification of the liquid colloid may lead to the formation of a coagulated liquid colloid which has a yogurt-like consistency.

Bacterial acidification of the liquid colloid in yogurt production may be performed at a temperature of 30° C. to 55° C. In some cases, bacterial acidification of the liquid colloid may be performed at temperature of at least 30° C. Bacterial acidification of the liquid colloid may be performed at temperature of at most 55° C. Bacterial acidification of the liquid colloid may be performed at temperature of about 30° C. to 35° C., 30° C. to 40° C., 30° C. to 45° C., 30° C. to 30° C. to 55° C., 35° C. to 40° C., 35° C. to 45° C., 35° C. to 50° C., 35° C. to 55° C., 40° C. to 45° C., to 50° C., 40° C. to 55° C., 45° C. to 50° C., 45° C. to 55° C., or 50° C. to 55° C. Bacterial acidification of the liquid colloid may be performed at temperature of about 30° C., 35° C., 40° C., 50° C., or 55° C. Bacterial acidification of the liquid colloid may be performed at temperature of at least 30° C., at least 35° C., at least 40° C., at least 45° C. or at least 50° C. Bacterial acidification of the liquid colloid may be performed at temperature of at most 35° C., at most at most 45° C., at most 50° C., or at most 55° C. In some cases, bacterial acidification may be performed at a temperature of 30° C. to 55° C. for at least 1 hour. In some cases, bacterial acidification may be performed at a temperature of 30° C. to 55° C. for at least 2 hours, at least 3 hours, at least 4 hours, at least 5 hours at least 6 hours, at least 8 hours, at least 10 hours or at least 12 hours. In some cases, bacterial acidification may be performed at a temperature of to 55° C. for at most 1 hour. In some cases, bacterial acidification may be performed at a temperature of 30° C. to 55° C. for at most 2 hours, at most 3 hours, at most 4 hours, at most 5 hours, at most 6 hours, at most 8 hours, at most 10 hours or at most 12 hours.

Alternatively, bacterial acidification may be performed at a lower temperature of to 30° C. Bacterial acidification of the liquid colloid may be performed at temperature of at least 15° C. Bacterial acidification of the liquid colloid may be performed at temperature of at most 30° C. Bacterial acidification of the liquid colloid may be performed at temperature of about 15° C. to 17° C., 15° C. to 20° C., 15° C. to 22° C., 15° C. to 25° C., 15° C. to 27° C., 15° C. to 30° C., 17° C. to 20° C., 17° C. to 22° C., 17° C. to 25° C., 17° C. to 27° C., 17° C. to 30° C., 20° C. to 22° C., 20° C. to 25° C., 20° C. to 27° C., 20° C. to 30° C., 22° C. to 25° C., 22° C. to 27° C., 22° C. to 30° C., 25° C. to 27° C., 25° C. to 30° C., or 27° C. to 30° C. Bacterial acidification of the liquid colloid may be performed at temperature of about 15° C., 17° C., 20° C., 22° C., 25° C., 27° C., or 30° C. Bacterial acidification of the liquid colloid may be performed at temperature of at least 15° C., at least 17° C., at least 20° C., at least 22° C., at least 25° C. or at least 27° C. Bacterial acidification of the liquid colloid may be performed at temperature of at most 17° C., at most 20° C., at most 22° C., at most 25° C., at most 27° C., or at most 30° C. In some cases, bacterial acidification may be performed at a temperature of 15° C. to 30° C. for at least 10 hours. In some cases, bacterial acidification may be performed at a temperature of 15° C. to 30° C. for at least 10 hours, at least 12 hours, at least 14 hours, at least 16 hours at least 18 hours, at least 20 hours, at least 22 hours or at least 24 hours. In some cases, bacterial acidification may be performed at a temperature of 15° C. to 30° C. for at most 24 hours. In some cases, bacterial acidification may be performed at a temperature of 15° C. to 30° C. for at most 12 hours, at most 14 hours, at most 16 hours, at most 18 hours, at most 20 hours, at most 22 hours or at most 24 hours.

Similar to cheese formation, a coagulated liquid colloid for yogurt formation may comprise other components such as sugars, fats, stabilizers and flavouring agents.

The concentration of fat in the yogurt product made from liquid colloid may be 0% to 12%. The yogurt product made from liquid colloid may comprise less than 1% fat, or in some cases no fats. The concentration of fat in the yogurt product made from liquid colloid may be at most 12%. The concentration of fat in the yogurt product made from liquid colloid may be 1% to 2%, 1% to 5%, 1% to 7%, 1% to 10%, 1% to 12%, 2% to 5%, 2% to 7%, 2% to 10%, 2% to 12%, 5% to 7%, 5% to 10%, 5% to 12%, 7% to 10%, 7% to 12%, or 10% to 12%. The concentration of fat in the yogurt product made from liquid colloid may be about 1%, 2%, 5%, 7%, 10%, or 12%. The concentration of fat in the yogurt product made from liquid colloid may be at least 1%, at least 2%, at least 5%, at least 7% or at least 10%. The concentration of fat in the yogurt product made from liquid colloid may be at most 2%, at most 5%, at most 7%, at most 10%, or at most 12%. Fats may be emulsified into liquid colloid (e.g., comprising micelles formed with alpha and kappa casein and salt) using sonication or high-pressure homogenization process. An emulsifier such as soy lecithin or xanthan gum may be used to secure a stable emulsion

The texture of a yogurt made by methods described herein may be comparable to the texture of a similar type of yogurt made using animal-obtained dairy proteins, such as yogurt made from animal milk. Texture of a yogurt made using a hybrid micelle composition may be comparable to the texture of a similar type of yogurt made using animal-obtained dairy proteins from any of the species whose caseins were used in the formation of the micelle. Texture of a yogurt may be tested using a trained panel of human subjects or machines such as a texture analyzer.

The taste of a yogurt made by methods described herein may be comparable to a similar type of yogurt made using animal-obtained dairy proteins. Taste of a yogurt made using a hybrid micelle composition may be comparable to the taste of a similar type of yogurt made using animal-obtained dairy proteins from any of the species whose caseins were used in the formation of the micelle. Taste of a yogurt may be tested using a trained panel of human subjects.

In some embodiments, dairy-like products may be produced using hybrid micelles or micelle like compositions described herein. Dairy-like products which can be made using the micelles and liquid colloids described herein may include milk, cream, milkshakes, creamers, ice cream, condensed milk, yogurt or cheese. Cheese analogues or cheese-like products which do not come from real curd or were not made via coagulation of a liquid colloid may also be made using the hybrid micelles or micelle like compositions described herein. In some cases, the dairy-product made using micelles or hybrid micelles may be a coagulated colloid composition. The coagulated colloid composition may be cheese curd.

### Applications of Hybrid Micelles or Liquid Colloids Described Herein

Provided herein are some non-limiting, exemplary applications, such as consumable compositions, of the hybrid micelles and liquid colloids described herein.

A. Pasta-Filata Cheese

In some embodiments, hybrid micelles and/or liquid colloids described herein may be used to produce a pasta-filata type cheese product (also referred to as a pasta-filata type cheese analog). The pasta-filata cheese product produced using hybrid micelles may have the properties found in pasta-filata cheese made using dairy-obtained milk or dairy-obtained proteins.

In some cases, a stretch of a pasta-filata cheese product may be comparable to or better than the stretch of a similar type of cheese made using animal-obtained dairy proteins, such as cheese made from animal milk. The stretch of a cheese can be measured by pulling the heated cheese until the breakage point and measuring the length of pulled strands or other conventional methods used in the art. In some cases, a melt of a pasta-filata cheese product may be comparable to or better than the melt of a similar type of cheese made using animal-obtained dairy proteins, such as cheese made from animal milk. Melt of a cheese can be measured using tests such as a Schreiber test or a similar test. In some cases, a firmness of a pasta-filata cheese product may be comparable to or better than the firmness of a similar type of cheese made using animal-obtained dairy proteins, such as cheese made from animal milk.

In some embodiments, a pasta-filata cheese product may be produced using a hybrid micelle comprising a mixture of an alpha casein and a kappa casein. In some cases, the alpha casein in a pasta-filata cheese may be a bovine alpha casein. The alpha casein in a pasta-filata cheese may be a native or recombinantly produced alpha casein. The alpha casein in a pasta-filata cheese may be a native or recombinantly produced alpha-S1-casein. In some cases, the kappa casein in a pasta-filata cheese may be a bovine kappa casein. In some cases, the kappa casein in a pasta-filata cheese may be a cow or buffalo kappa casein. In some cases, the kappa casein in a pasta-filata cheese may be a sheep kappa casein. In some cases, the kappa casein in a pasta-filata cheese may be a goat kappa casein. The kappa casein in a pasta-filata cheese may be a native or recombinantly produced kappa casein. Other forms of alpha/kappa casein described herein can also be used to produce a pasta-filata cheese.

In some embodiments, a ratio of alpha casein to kappa casein in a hybrid micelle to be used to make a pasta-filata cheese may be about 2.5:1, 3:1, 3.5:1, 4:1, 4.5:1, 5:1 or 5.5:1. In some cases, a ratio of alpha casein to kappa casein in a hybrid micelle to be used to make a pasta-filata cheese may be from 3:1 to 5:1 or 4:1 to 5:1.

In one example, a hybrid micelle to be used to make a pasta-filata cheese may comprise a bovine alpha casein and a sheep kappa casein, such as a bovine alpha-S1-casein and a sheep kappa casein. In such an example, a ratio of alpha casein to kappa casein may be from 3:1 to 5:1 or 4:1 to 5:1.

In another example, a hybrid micelle to be used to make a pasta-filata cheese may comprise a cow alpha casein and a buffalo kappa casein. The alpha and/or the kappa casein in such an example may have native PTMs or they may be altered forms of the alpha or kappa casein proteins. In such an example, a ratio of alpha casein to kappa casein may be from 3:1 to 4:1.

In yet another example, a hybrid micelle to be used to make a pasta-filata cheese may comprise a bovine alpha casein and a goat kappa casein. The alpha and/or the kappa casein in such an example may have native PTMs or they may be altered forms of the alpha or kappa casein proteins. In such an example, a ratio of alpha casein to kappa casein may be from 3:1 to 4:1.

In yet another example, a hybrid micelle to be used to make a pasta-filata cheese may comprise a bovine alpha casein and a sheep kappa casein. The alpha and/or the kappa casein in such an example may have native PTMs or they may be altered forms of the alpha or kappa casein proteins. In such an example, a ratio of alpha casein to kappa casein may be from 3:1 to 4:1.

In one example, a liquid colloid comprising hybrid micelles may be used to produce a pasta-filata cheese. A pH of the acidified liquid colloid in such an example may be from 5-In such an example, conditions to form a curd and a pasta-filata cheese may be similar to conditions described in Examples 7 and 9 herein.

B. Goat-Like Soft and Spreadable Cheese

In some embodiments, hybrid micelles and/or liquid colloids described herein may be used to produce a soft and spreadable type cheese product (also referred to as a soft and spreadable cheese analog). A soft and spreadable cheese product may be similar to a goat cheese. The soft and spreadable cheese product produced using hybrid micelles may have the properties found in soft and spreadable cheese made using dairy-obtained milk or dairy-obtained proteins.

In some cases, a spreadability of a soft and spreadable cheese product may be comparable to or better than the spreadability of a similar type of cheese made using animal-obtained dairy proteins, such as cheese made from animal milk. In some cases, a melt of a soft and spreadable cheese product may be comparable to or better than the melt of a similar type of cheese made using animal-obtained dairy proteins, such as cheese made from animal milk. In some cases, a softness of a soft and spreadable cheese product may be comparable to or better than the softness of a similar type of cheese made using animal-obtained dairy proteins, such as cheese made from animal milk. Softness or hardness can be measured using texture analyzer. In some cases, a moistness of a soft and spreadable cheese product may be comparable to or better than the moistness of a similar type of cheese made using animal-obtained dairy proteins, such as cheese made from animal milk. Moistness can be measured by moisture content of a cheese. In some cases, a firmness of a soft and spreadable cheese product may be comparable to or better than the firmness of a similar type of cheese made using animal-obtained dairy proteins, such as cheese made from animal milk. In some cases, a soft and spreadable cheese product is not stretchable or not substantially stretchable. In some cases, the curd made from the hybrid micelles and used to produce a soft and spreadable cheese forms loose curds.

In some embodiments, a soft and spreadable cheese product may be produced using a hybrid micelle comprising a mixture of an alpha casein and a kappa casein. In some cases, the alpha casein in a soft and spreadable cheese product may be a bovine alpha casein. The alpha casein in a soft and spreadable cheese product may be a native or recombinantly produced alpha casein. The alpha casein in a soft and spreadable cheese may be an alpha-S1-casein. In some cases, the kappa casein in a soft and spreadable cheese product may be a goat kappa casein. The kappa casein in a soft and spreadable cheese product may be a native or recombinantly produced kappa casein. Other forms of alpha/kappa casein described herein can also be used to produce a soft and spreadable cheese product. The alpha and/or the kappa casein in such an example may have native PTMs or they may be altered forms of the alpha or kappa casein proteins. In some cases, the alpha or kappa caseins in a soft and spreadable cheese may lack any PTMs.

In some embodiments, a ratio of alpha casein to kappa casein in a hybrid micelle to be used to make a soft and spreadable cheese product may be about 2.5:1, 3:1, 3.5:1, 4:1, 4.5:1, or 5.5:1. In some cases, a ratio of alpha casein to kappa casein in a hybrid micelle to be used to make a soft and spreadable cheese product may be from 3:1 to 5:1 or 4:1 to 5:1.

In one example, a liquid colloid comprising hybrid micelles may be used to produce a soft and spreadable cheese product. A pH of the acidified liquid colloid in such an example may be from 5-5.5. In such an example, conditions to form a curd and a soft and spreadable cheese product may be similar to conditions described in Examples 7 and 9 herein.

C. Crumbly Cheese

In some embodiments, hybrid micelles and/or liquid colloids described herein may be used to produce a crumbly type cheese product (also referred to as a crumbly cheese analog). A crumbly cheese product may be similar to a feta cheese. The crumbly cheese product produced using hybrid micelles may have the properties found in crumbly cheese made using dairy-obtained milk or dairy-obtained proteins.

In some cases, a melt of a crumbly cheese product may be comparable to or better than the melt of a similar type of cheese made using animal-obtained dairy proteins, such as cheese made from animal milk. In some cases, a crumbly cheese is not stretchable or not substantially stretchable.

In some embodiments, a crumbly cheese product may be produced using a hybrid micelle comprising a mixture of an alpha casein and a kappa casein. In some cases, the alpha casein in a crumbly cheese product may be a bovine alpha casein. In some cases, the alpha casein in a crumbly cheese product may be an alpha-S1-casein. The alpha casein in a crumbly cheese product may be a native or recombinantly produced alpha casein. In some cases, the kappa casein in a crumbly cheese product may be a bovine kappa casein. In one example, the kappa casein is a truncated bovine kappa casein lacking 16 amino acid residues from the C-terminus. The kappa casein in a crumbly cheese product may be a native or recombinantly produced kappa casein. Other forms of alpha/kappa casein described herein can also be used to produce a crumbly cheese.

In some embodiments, a ratio of alpha casein to kappa casein in a hybrid micelle to be used to make a crumbly cheese product may be about 2.5:1, 3:1, 3.5:1 or 4:1. In some embodiments, a ratio of alpha casein to kappa casein in a hybrid micelle to be used to make a crumbly cheese product may be from 2.5:1 to 3:1, 3:1 to 3.5:1 or 3.5:1 to 4:1.

In one example, a liquid colloid comprising hybrid micelles may be used to produce a crumbly cheese product. A pH of the acidified liquid colloid in such an example may be from 5.7-6.0. In such an example, conditions to form a curd and a crumbly cheese product may be similar to conditions described in Example 11 herein.

D. Soft Cheese

In some embodiments, hybrid micelles and/or liquid colloids described herein may be used to produce a soft type cheese product (also referred to as a soft cheese analog). A soft cheese product may be similar to a cottage cheese or other fresh cheeses. The soft cheese product produced using hybrid micelles may have the properties found in soft cheese made using dairy-obtained milk or dairy-obtained proteins.

In some cases, a spreadability of a soft cheese product may be comparable to or better than the spreadability of a similar type of cheese made using animal-obtained dairy proteins, such as cheese made from animal milk. In some cases, a soft cheese product is not stretchable and does not melt. In some cases, a soft cheese product is not substantially stretchable and does not substantially melt under the same conditions whereby a cheese made from animal milk would melt. In some cases, the curd used to produce a soft cheese forms runny curds with an increased water holding capacity.

In some embodiments, a soft cheese product may be produced using a hybrid micelle comprising a mixture of an alpha casein and a kappa casein. In some cases, the alpha casein in a soft cheese product may be a bovine alpha casein. In some cases, the alpha casein in a soft cheese product may be an alpha-S1-casein. In one example, the alpha casein in a soft cheese product may be a F24 truncated bovine alpha casein (SEQ ID NO: 6). The alpha casein in a soft cheese product may be a native or recombinantly produced alpha casein. In some cases, the kappa casein in a soft cheese product may be a bovine kappa casein. The kappa casein in a soft cheese product may be a native or recombinantly produced kappa casein. Other forms of alpha/kappa casein described herein can also be used to produce a soft cheese product.

In some embodiments, a ratio of alpha casein to kappa casein in a hybrid micelle to be used to make a soft cheese product may be about 2.5:1, 3:1 or 3.5:1. In some embodiments, a ratio of alpha casein to kappa casein in a hybrid micelle to be used to make a soft cheese product may be from 2.5:1 to 3:1 or 3:1 to 3.5:1.

In one example, a liquid colloid comprising hybrid micelles may be used to produce a soft cheese product. A pH of the acidified liquid colloid in such an example may be from 5-In such an example, conditions to form a curd and a soft cheese product may be similar to conditions described in Example 10 herein.

E. Yogurt

In some embodiments, hybrid micelles and/or liquid colloids described herein may be used to produce a yogurt product (also referred to as a yogurt analog). A yogurt may be similar to a dairy-obtained yogurt. The yogurt product produced using hybrid micelles may have the properties found in yogurt made using dairy-obtained milk or dairy-obtained proteins.

In some cases, a consistency of a yogurt product may be comparable to or better than the consistency of a similar type of yogurt made using animal-obtained dairy proteins, such as yogurt made from animal milk. In some cases, the curd used to produce a yogurt product forms runny curds with an increased water holding capacity.

In some embodiments, a yogurt product may be produced using a hybrid micelle comprising a mixture of an alpha casein and a kappa casein. In some cases, the alpha casein in a yogurt product may be a bovine alpha casein. In some cases, the alpha casein in a yogurt product may be an alpha-S1-casein. In one example, the alpha casein in a yogurt product may be a F24 truncated bovine alpha casein (SEQ ID NO: 6). The alpha casein in a yogurt product may be a native or recombinantly produced alpha casein. In some cases, the kappa casein in a yogurt product may be a bovine kappa casein. The kappa casein in a yogurt product may be a native or recombinantly produced kappa casein. Other forms of alpha/kappa casein described herein can also be used to produce a yogurt. composition

In some embodiments, a ratio of alpha casein to kappa casein in a hybrid micelle to be used to make a yogurt product may be about 2.5:1, 3:1 or 3.5:1. In some embodiments, a ratio of alpha casein to kappa casein in a hybrid micelle to be used to make a yogurt product may be from 2.5:1 to 3:1 or 3:1 to 3.5:1.

In one example, a liquid colloid comprising hybrid micelles may be used to produce a yogurt product. A pH of the liquid colloid in such an example may be from 4.2-5.5. In such an example, conditions to form a yogurt product may be similar to conditions described in Example 10 herein. For instance, a culture of yogurt producing microorganisms may be added to the micellar colloid formed in Example 10 to produce a composition similar to a yogurt.

F. Hard Cheeses

In some embodiments, hybrid micelles and/or liquid colloids described herein may be used to produce a hard cheese product (also referred to as a hard cheese analog) or a very-hard cheese product. A hard cheese product may be similar to a swiss cheese or a cheddar cheese. A very hard cheese may be similar to a parmesan cheese. The hard/very-hard cheese product produced using hybrid micelles (or aggregates) may have the properties found in hard/very hard cheese made using dairy-obtained milk or dairy-obtained proteins.

In some cases, a hard/very hard cheese product is not stretchable but is able to melt. In some cases, a hard/very hard cheese product melts under the same conditions whereby a cheese made from animal milk would melt.

In some embodiments, a hard/very hard cheese product may be produced using a hybrid micelle comprising a mixture of an alpha casein and a kappa casein. In some cases, the alpha casein in a hard/very hard cheese product may be a bovine alpha casein. In some cases, the alpha casein in a hard/very hard cheese product may be an alpha-S1-casein. The alpha casein in a hard/very hard cheese product may be a native or recombinantly produced alpha casein. In some cases, the kappa casein in a hard/very hard cheese product may be a sheep kappa casein. The kappa casein in a hard/very hard cheese product may be a native or recombinantly produced kappa casein. Other forms of alpha/kappa casein described herein can also be used to produce a hard/very hard cheese product.

### Recombinant Expression

One or more proteins used in the formation of dairy-like compositions may be produced recombinantly. In some cases, alpha (e.g., alpha-S1 and/or alpha-S2), beta, and kappa casein are each produced recombinantly. In some cases, one of alpha, beta, and kappa casein are produced recombinantly. In some cases, more than one of alpha, beta, and kappa casein are produced recombinantly. In some cases, a truncation of a casein, such as a N- or C-terminal truncation of alpha, beta, or kappa casein is produced recombinantly.

Alpha-S1 and/or alpha-S2 casein can have an amino acid sequence from any species. For example, recombinant alpha casein may have an amino acid sequence of cow, sheep, goat, buffalo, horse, deer or camel alpha casein. Alpha casein nucleotide sequence may be codon-optimized for increased efficiency of production. Exemplary alpha casein protein sequences are provided in Table 1 below. Recombinant alpha casein can be a non-naturally occurring variant of an alpha casein. Such variant can comprise one or more amino acid insertions, deletions, or substitutions relative to a native alpha casein sequence.

Such a variant can have at least 85%, at least 90%, at least 95%, at least 96%, at least 97%, at least 98%, or at least 99% sequence identity to SEQ ID NOs: 1-47. In some cases, a variant may be a truncated form of the alpha-S1-casein protein such as one with at least 85%, at least 90%, at least 95%, at least 96%, at least 97%, at least 98%, or at least 99% sequence identity to SEQ ID NO.: 3.

Beta casein can have an amino acid sequence from any species. For example, recombinant beta casein may have an amino acid sequence of cow, human, sheep, goat, buffalo, bison, horse, deer or camel beta casein. Beta casein nucleotide sequence may be codon-optimized for increased efficiency of production. Exemplary beta casein amino acid sequences are provided in Table 1 below. Recombinant beta casein can be a non-naturally occurring variant of a beta casein. Such variant can comprise one or more amino acid insertions, deletions, or substitutions relative to a native beta sequence. Such a variant can have at least 85%, at least 90%, at least 95%, at least 96%, at least 97%, at least 98%, or at least 99% sequence identity to SEQ ID NO: 71-85.

Kappa casein can have an amino acid sequence from any species. For example, recombinant kappa casein may have an amino acid sequence of cow, human, sheep, goat, buffalo, bison, horse, deer or camel kappa casein. Kappa casein nucleotide sequence may be codon-optimized for increased efficiency of production. Exemplary kappa casein amino acid sequences are provided in Table 1 below. Recombinant kappa casein can be a non-naturally occurring variant of a kappa casein. Such variant can comprise one or more amino acid insertions, deletions, or substitutions relative to a native kappa sequence.

Such a variant can have at least 85%, at least 90%, at least 95%, at least 96%, at least 97%, at least 98%, or at least 99% sequence identity to SEQ ID NOs: 48-70. In some cases, a variant may be a truncated form of the kappa casein protein such as one with at least 85%, at least 90%, at least 95%, at least 96%, at least 97%, at least 98%, or at least 99% sequence identity to SEQ ID NO.: 51.

A recombinant casein protein, such as alpha, beta or kappa casein, is recombinantly expressed in a host cell. As used herein, a “host” or “host cell” denotes any protein production host selected or genetically modified to produce a desired product. Exemplary hosts include bacteria, yeast, fungi, plant insect and mammalian cells. In some cases, a bacterial host cell such as Lactococcus lactis, Bacillus subtilis or Escherichia coli may be used to produce alpha and/or kappa casein proteins. Other host cells include bacterial host such as, but not limited to, Lactococci sp., Lactococcus lactis, Bacillus subtilis, Bacillus amyloliquefaciens, Bacillus licheniformis and Bacillus megaterium, Brevibacillus choshinensis, Mycobacterium smegmatis, Rhodococcus erythropolis and Corynebacterium glutamicum, Lactobacilli sp., Lactobacillus fermentum, Lactobacillus casei, Lactobacillus acidophilus, Lactobacillus plantarum and Synechocystis sp. 6803.

In some embodiments, alpha casein protein, kappa casein protein or both alpha and kappa casein proteins are produced recombinantly in a host cell. Alpha and kappa caseins may be produced in the same host cell. Alternatively, alpha and kappa casein may be produced in different host cells. Expression of a target protein can be provided by an expression vector, a plasmid, a nucleic acid integrated into the host genome or other means. For example, a vector for expression can include: (a) a promoter element, (b) a signal peptide, (c) a heterologous casein sequence, and (d) a terminator element.

Expression vectors that can be used for expression of casein include those containing an expression cassette with elements (a), (b), (c) and (d). In some embodiments, the signal peptide (c) need not be included in the vector. In some cases, a signal peptide may be part of the native signal sequence of the casein protein, for instance, the protein may comprise a native signal sequence as bolded in SEQ ID NOs: 1, 12, 15, 18, 21, 24, 27, 30, 33, 36, 39, 42, 48, 53, 56, 59, 62, 65, 68 or 71. In some cases, the vector may comprise a mature protein sequence, as exemplified in SEQ ID NOs: 2-11, 13, 14, 16, 17, 19, 20, 22, 23, 25, 26, 28, 29, 31, 32, 34, 35, 37, 38, 40, 41, 43, 44, 46, 47, 49, 50, 51, 52, 54, 55, 57, 58, 60, 61, 63, 64, 66, 67, 69, 70 or 72-85 with a heterologous signal sequence. In some cases, the vector may comprise a mature protein sequence, as exemplified in SEQ ID NOs: 2-11, 13, 14, 16, 17, 19, 22, 23, 25, 26, 28, 29, 31, 32, 34, 35, 37, 38, 40, 41, 43, 44, 46, 47, 49, 50, 51, 52, 54, 55, 57, 58, 60, 61, 63, 64, 66, 67, 69, 70 or 72-85, and a methionine may be encoded in the vector immediately upstream and in-frame with the coding sequence for the mature protein sequence. In general, the expression cassette is designed to mediate the transcription of the transgene when integrated into the genome of a cognate host microorganism or when present on a plasmid or other replicating vector maintained in a host cell.

To aide in the amplification of the vector prior to transformation into the host microorganism, a replication origin (e) may be contained in the vector. To aide in the selection of microorganism stably transformed with the expression vector, the vector may also include a selection marker (f). The expression vector may also contain a restriction enzyme site (g) that allows for linearization of the expression vector prior to transformation into the host microorganism to facilitate the expression vectors stable integration into the host genome. In some embodiments the expression vector may contain any subset of the elements (b), (e), (f), and (g), including none of elements (b), (e), (f), and (g). Other expression elements and vector element known to one of skill in the art can be used in combination or substituted for the elements described herein.

Gram positive bacteria (such as Lactococcus lactis and Bacillus subtilis) may be used to secrete target proteins into the media, and gram-negative bacteria (such as Escherichia coli) may be used to secrete target proteins into periplasm or into the media. In some embodiments, the bacterially-expressed proteins expressed may not have any post-translational modifications (PTMs), which means they are not glycosylated and/or may not be phosphorylated.

Target casein proteins may be expressed and produced in L. lactis both in a nisin-inducible expression system (regulated by PnisA promoter), lactate-inducible expression system (regulated by P170 promoter) or other similar inducible systems, as well as a constitutively expressed system (regulated by P secA promoter), wherein both are in a food-grade selection strain, such as NZ3900 using vector pNZ8149 (lacF gene supplementation/rescue principle). The secretion of functional proteins may be enabled by the signal peptide of Usp45 (SP(usp45)), the major Sec-dependent protein secreted by L. lactis. For example, alpha-S1-casein and kappa casein may be co-expressed or individually expressed in L. lactis using a synthetic operon, where the gene order is kappa casein-alpha-S1-casein.

Bacillus subtilis Design

B. subtilis, unlike L. lactis, has multiple intracellular and extracellular proteases, which may interfere with protein expression. In some embodiments, B. subtilis strains are modified to reduce the type and amount of intracellular and/or extracellular proteases, for example strains which have deletions for 7 (K07) and 8 (WB800N) proteases, respectively, may be used.

In order to drive the recombinant protein secretion, the signal peptide of amyQ, alpha-amylase of Clostridium thermocellum may be used. Additionally, native casein signal peptide sequences may be expressed heterologously in B. subtilis. Each casein protein has its own signal peptide sequence and may be used in the system. The signal proteins may be cross-combined with the casein proteins. The pHT01 vector may be used as a transformation and expression shuttle for inducible protein expression in B. subtilis. The vector is based on the strong σ^{A}-dependent promoter preceding the groES-groELoperon of B. subtilis, which has been converted into an efficiently controllable (IPTG-inducible) promoter by addition of the lac operator. pHT01 is an E. coli/B. subtilis shuttle vector that provides ampicillin resistance to E. coli and chloramphenicol resistance to B. subtilis.

Untagged and tagged versions of caseins may be expressed, whereby a small peptide tag such as His or StrepII tag, sequence or fusion protein such as GST, MBP or SUMO is placed N- or C-terminally to casein without the secretion signal peptide. Given secondary structures of kappa, alpha-S1, and alpha-S2-casein, tagging may be less disruptive at N-terminal of kappa casein, whereby alpha-S1 casein can likely be tagged at both termini. However, other tags may be used.

TABLE 1
Sequences

| SEQ | | |
|---|---|---|
| ID | | |
| No. | Name | Sequence |
| 1 | Bovine Alpha-S1 Casein | MKLLILTCLVAVALARPKHPIKHQGLPQEVLNENLLRFFVAP |
| | | FPEVFGKEKVNELSKDIGSESTEDQAMEDIKQMEAESISSSEEI |
| | | VPNSVEQKHIQKEDVPSERYLGYLEQLLRLKKYKVPQLEIVPN |
| | | SAEERLHSMKEGIHAQQKEPMIGVNQELAYFYPELFRQFYQL |
| | | DAYPSGAWYYVPLGTQYTDAPSFSDIPNPIGSENSEKTTMPLW |
| 2 | Bovine Alpha-S1 Casein | RPKHPIKHQGLPQEVLNENLLRFFVAPFPEVFGKEKVNELSKDI |
| | Mature Protein | GSESTEDQAMEDIKQMEAESISSSEEIVPNSVEQKHIQKEDVPS |
| | | ERYLGYLEQLLRLKKYKVPQLEIVPNSAEERLHSMKEGIHAQQ |
| | | KEPMIGVNQELAYFYPELFRQFYQLDAYPSGAWYYVPLGTQY |
| | | TDAPSFSDIPNPIGSENSEKTTMPLW |
| 3 | Bovine Alpha-S1 Casein | MRPKHPIKHQGLPQEVLNENLLRFFVAPFPEVFGKEKVNELSK |
| | Mature Protein with | DIGSESTEDQAMEDIKQMEAESISSSEEIVPNSVEQKHIQKEDV |
| | Methionine | PSERYLGYLEQLLRLKKYKVPQLEIVPNSAEERLHSMKEGIHA |
| | | QQKEPMIGVNQELAYFYPELFRQFYQLDAYPSGAWYYVPLGT |
| | | QYTDAPSFSDIPNPIGSENSEKTTMPLW |
| 4 | Bovine alpha-S1-casein | FFVAPFPEVFGKEKVNELSKDIGSESTEDQAMEDIKQMEAESIS |
| | (B. Taurus) aa 23-199 | SSEEIVPNSVEQKHIQKEDVPSERYLGYLEQLLRLKKYKVPQL |
| | | EIVPNSAEERLHSMKEGIHAQQKEPMIGVNQELAYFYPELFRQ |
| | | FYQLDAYPSGAWYYVPLGTQYTDAPSFSDIPNPIGSENSGKTT |
| | | MPLW |
| 5 | Bovine alpha-S1-casein | MFFVAPFPEVFGKEKVNELSKDIGSESTEDQAMEDIKQMEAES |
| | (B. Taurus) aa 23-199 with | ISSSEEIVPNSVEQKHIQKEDVPSERYLGYLEQLLRLKKYKVPQ |
| | Methionine | LEIVPNSAEERLHSMKEGIHAQQKEPMIGVNQELAYFYPELFR |
| | | QFYQLDAYPSGAWYYVPLGTQYTDAPSFSDIPNPIGSENSGKT |
| | | TMPLW |
| 6 | Bovine alpha-S1-casein | FVAPFPEVFGKEKVNELSKDIGSESTEDQAMEDIKQMEAESISS |
| | (B. Taurus) aa 24-199 | SEEIVPNSVEQKHIQKEDVPSERYLGYLEQLLRLKKYKVPQLEI |
| | | VPNSAEERLHSMKEGIHAQQKEPMIGVNQELAYFYPELFRQFY |
| | | QLDAYPSGAWYYVPLGTQYTDAPSFSDIPNPIGSENSGKTTMP |
| | | LW |
| 7 | Bovine alpha-S1-casein | MFVAPFPEVFGKEKVNELSKDIGSESTEDQAMEDIKQMEAESI |
| | (B. Taurus) aa 24-199 with | SSSEEIVPNSVEQKHIQKEDVPSERYLGYLEQLLRLKKYKVPQ |
| | Methionine | LEIVPNSAEERLHSMKEGIHAQQKEPMIGVNQELAYFYPELFR |
| | | QFYQLDAYPSGAWYYVPLGTQYTDAPSFSDIPNPIGSENSGKT |
| | | TMPLW |
| 8 | Bovine alpha-S1-casein | VAPFPEVFGKEKVNELSKDIGSESTEDQAMEDIKQMEAESISSS |
| | (B. Taurus) aa 25-199 | EEIVPNSVEQKHIQKEDVPSERYLGYLEQLLRLKKYKVPQLEI |
| | | VPNSAEERLHSMKEGIHAQQKEPMIGVNQELAYFYPELFRQFY |
| | | QLDAYPSGAWYYVPLGTQYTDAPSFSDIPNPIGSENSGKTTMP |
| | | LW |
| 9 | Bovine alpha-S1-casein | MVAPFPEVFGKEKVNELSKDIGSESTEDQAMEDIKQMEAESIS |
| | (B. Taurus) aa 25-199 with | SSEEIVPNSVEQKHIQKEDVPSERYLGYLEQLLRLKKYKVPQL |
| | Methionine | EIVPNSAEERLHSMKEGIHAQQKEPMIGVNQELAYFYPELFRQ |
| | | FYQLDAYPSGAWYYVPLGTQYTDAPSFSDIPNPIGSENSGKTT |
| | | MPLW |
| 10 | Bovine alpha-S1-casein | APFPEVFGKEKVNELSKDIGSESTEDQAMEDIKQMEAESISSSE |
| | (B. Taurus) aa 26-199 | EIVPNSVEQKHIQKEDVPSERYLGYLEQLLRLKKYKVPQLEIVP |
| | | NSAEERLHSMKEGIHAQQKEPMIGVNQELAYFYPELFRQFYQ |
| | | LDAYPSGAWYYVPLGTQYTDAPSFSDIPNPIGSENSGKTTMPL |
| | | W |
| 11 | Bovine alpha-S1-casein | MAPFPEVFGKEKVNELSKDIGSESTEDQAMEDIKQMEAESISSS |
| | (B. Taurus) aa 26-199 with | EEIVPNSVEQKHIQKEDVPSERYLGYLEQLLRLKKYKVPQLEI |
| | Methionine | VPNSAEERLHSMKEGIHAQQKEPMIGVNQELAYFYPELFRQFY |
| | | QLDAYPSGAWYYVPLGTQYTDAPSFSDIPNPIGSENSGKTTMP |
| | | LW |
| 12 | Ovine Alpha-S1-casein | MKLLILTCLVAVALARPKHPIKHQGLSSEVLNENLLRFVVAP |
| | | FPEVFRKENINELSKDIGSESIEDQAMEDAKQMKAGSSSSSEEI |
| | | VPNSAEQKYIQKEDVPSERYLGYLEQLLRLKKYNVPQLEIVPK |
| | | SAEEQLHSMKEGNPAHQKQPMIAVNQELAYFYPQLFRQFYQL |
| | | DAYPSGAWYYLPLGTQYTDAPSFSDIPNPIGSENSGKITMPLW |
| 13 | Ovine Alpha-S1-casein | RPKHPIKHQGLSSEVLNENLLRFVVAPFPEVFRKENINELSKDI |
| | Mature protein | GSESIEDQAMEDAKQMKAGSSSSSEEIVPNSAEQKYIQKEDVP |
| | | SERYLGYLEQLLRLKKYNVPQLEIVPKSAEEQLHSMKEGNPA |
| | | HQKQPMIAVNQELAYFYPQLFRQFYQLDAYPSGAWYYLPLGT |
| | | QYTDAPSFSDIPNPIGSENSGKITMPLW |
| 14 | Ovine Alpha-S1-casein | MRPKHPIKHQGLSSEVLNENLLRFVVAPFPEVFRKENINELSK |
| | Mature protein with | DIGSESIEDQAMEDAKQMKAGSSSSSEEIVPNSAEQKYIQKED |
| | Methionine | VPSERYLGYLEQLLRLKKYNVPQLEIVPKSAEEQLHSMKEGNP |
| | | AHOKQPMIAVNQELAYFYPQLFRQFYQLDAYPSGAWYYLPL |
| | | GTQYTDAPSFSDIPNPIGSENSGKITMPLW |
| 15 | Caprine Alpha-S1-casein | MKLLILTCLVAVALARPKHPINHRGLSPEVPNENLLRFVVAP |
| | | FPEVFRKENINELSKDIGSESTEDQAMEDAKQMKAGSSSSSEEI |
| | | VPNSAEQKYIQKEDVPSERYLGYLEQLLRLKKYNVPQLEIVPK |
| | | SAEEQLHSMKEGNPAHQKQPMIAVNQELAYFYPQLFRQFYQL |
| | | DAYPSGAWYYLPLGTQYTDAPSFSDIPNPIGSENSGKTTMPLW |
| 16 | Caprine Alpha-S1-casein | RPKHPINHRGLSPEVPNENLLRFVVAPFPEVFRKENINELSKDI |
| | Mature Protein | GSESTEDQAMEDAKQMKAGSSSSSEEIVPNSAEQKYIQKEDVP |
| | | SERYLGYLEQLLRLKKYNVPQLEIVPKSAEEQLHSMKEGNPA |
| | | HQKQPMIAVNQELAYFYPQLFRQFYQLDAYPSGAWYYLPLGT |
| | | QYTDAPSFSDIPNPIGSENSGKTTMPLW |
| 17 | Caprine Alpha-S1-casein | MRPKHPINHRGLSPEVPNENLLRFVVAPFPEVFRKENINELSKD |
| | Mature Protein with | IGSESTEDQAMEDAKQMKAGSSSSSEEIVPNSAEQKYIQKEDV |
| | Methionine | PSERYLGYLEQLLRLKKYNVPQLEIVPKSAEEQLHSMKEGNPA |
| | | HQKQPMIAVNQELAYFYPQLFRQFYQLDAYPSGAWYYLPLGT |
| | | QYTDAPSFSDIPNPIGSENSGKTTMPLW |
| 18 | Buffalo Alpha-S1-casein | MKLLILTCLVAVALARPKQPIKHQGLPQGVLNENLLRFFVAP |
| | | FPEVFGKEKVNELSTDIGSESTEDQAMEDIKQMEAESISSSEEIV |
| | | PISVEQKHIQKEDVPSERYLGYLEQLLRLKKYNVPQLEIVPNLA |
| | | EEQLHSMKEGIHAQQKEPMIGVNQELAYFYPQLFRQFYQLDA |
| | | YPSGAWYYVPLGTQYPDAPSFSDIPNPIGSENSEKTTMPLW |
| 19 | Buffalo Alpha-S1-casein | RPKQPIKHQGLPQGVLNENLLRFFVAPFPEVFGKEKVNELSTDI |
| | Mature Protein | GSESTEDQAMEDIKQMEAESISSSEEIVPISVEQKHIQKEDVPSE |
| | | RYLGYLEQLLRLKKYNVPQLEIVPNLAEEQLHSMKEGIHAQQ |
| | | KEPMIGVNQELAYFYPQLFRQFYQLDAYPSGAWYYVPLGTQY |
| | | PDAPSFSDIPNPIGSENSEKTTMPLW |
| 20 | Buffalo Alpha-S1-casein | MRPKQPIKHQGLPQGVLNENLLRFFVAPFPEVFGKEKVNELST |
| | Mature Protein with | DIGSESTEDQAMEDIKQMEAESISSSEEIVPISVEQKHIQKEDVP |
| | Methionine | SERYLGYLEQLLRLKKYNVPQLEIVPNLAEEQLHSMKEGIHAQ |
| | | QKEPMIGVNQELAYFYPQLFRQFYQLDAYPSGAWYYVPLGTQ |
| | | YPDAPSFSDIPNPIGSENSEKTTMPLW |
| 21 | Equine Alpha-S1-casein | MKLLILTCLVAVALARPKLPHRQPEIIQNEQDSREKVLKERKF |
| | | PSFALEYINELNRQRELLKEKQKDEHKEYLIEDPEQQESSSTSS |
| | | SEEVVPINTEQKRIPREDMLYQHTLEQLRRLSKYNQLQLQAIH |
| | | AQEQLIRMKENSQRKPMRVVNQEQAYFYLEPFQPSYQLDVYP |
| | | YAAWFHPAQIMQHVAYSPFHDTAKLIASENSEKTDIIPEW |
| 22 | Equine Alpha-S1-casein | RPKLPHRQPEIIQNEQDSREKVLKERKFPSFALEYINELNRQRE |
| | Mature Protein | LLKEKQKDEHKEYLIEDPEQQESSSTSSSEEVVPINTEQKRIPRE |
| | | DMLYQHTLEQLRRLSKYNQLQLQAIHAQEQLIRMKENSQRKP |
| | | MRVVNQEQAYFYLEPFQPSYQLDVYPYAAWFHPAQIMQHVA |
| | | YSPFHDTAKLIASENSEKTDIIPEW |
| 23 | Equine Alpha-S1-casein | MRPKLPHRQPEIIQNEQDSREKVLKERKFPSFALEYINELNRQR |
| | Mature Protein with | ELLKEKQKDEHKEYLIEDPEQQESSSTSSSEEVVPINTEQKRIPR |
| | Methionine | EDMLYQHTLEQLRRLSKYNQLQLQAIHAQEQLIRMKENSQRK |
| | | PMRVVNQEQAYFYLEPFQPSYQLDVYPYAAWFHPAQIMQHV |
| | | AYSPFHDTAKLIASENSEKTDIIPEW |
| 24 | Camel Alpha-S1-casein | MKLLILTCLVAVALARPKYPLRYPEVFQNEPDSIEEVLNKRKI |
| | | LELAVVSPIQFRQENIDELKDTRNEPTEDHIMEDTERKESGSSS |
| | | SEEVVSSTTEQKDILKEDMPSQRYLEELHRLNKYKLLQLEAIR |
| | | DQKLIPRVKLSSHPYLEQLYRINEDNHPQLGEPVKVVTQEQAY |
| | | FHLEPFPQFFQLGASPYVAWYYPPQVMQYIAHPSSYDTPEGIA |
| | | SEDGGKTDVMPQWW |
| 25 | Camel Alpha-S1-casein | RPKYPLRYPEVFQNEPDSIEEVLNKRKILELAVVSPIQFRQENID |
| | Mature Protein | ELKDTRNEPTEDHIMEDTERKESGSSSSEEVVSSTTEQKDILKE |
| | | DMPSQRYLEELHRLNKYKLLQLEAIRDQKLIPRVKLSSHPYLE |
| | | QLYRINEDNHPQLGEPVKVVTQEQAYFHLEPFPQFFQLGASPY |
| | | VAWYYPPQVMQYIAHPSSYDTPEGIASEDGGKTDVMPQWW |
| 26 | Camel Alpha-S1-casein | MRPKYPLRYPEVFQNEPDSIEEVLNKRKILELAVVSPIQFRQEN |
| | Mature Protein with | IDELKDTRNEPTEDHIMEDTERKESGSSSSEEVVSSTTEQKDIL |
| | Methionine | KEDMPSQRYLEELHRLNKYKLLQLEAIRDQKLIPRVKLSSHPY |
| | | LEQLYRINEDNHPQLGEPVKVVTQEQAYFHLEPFPQFFQLGAS |
| | | PYVAWYYPPQVMQYIAHPSSYDTPEGIASEDGGKTDVMPQW |
| | | W |
| 27 | Human Alpha-S1-casein | MRLLILTCLVAVALARPKLPLRYPERLQNPSESSEPIPLESREE |
| | | YMNGMNRQRNILREKQTDEIKDTRNESTQNCVVAEPEKMESS |
| | | ISSSSEEMSLSKCAEQFCRLNEYNQLQLQAAHAQEQIRRMNEN |
| | | SHVQVPFQQLNQLAAYPYAVWYYPQIMQYVPFPPFSDISNPT |
| | | AHENYEKNNVMLQW |
| 28 | Human Alpha-S1-casein | RPKLPLRYPERLQNPSESSEPIPLESREEYMNGMNRQRNILREK |
| | Mature Protein | QTDEIKDTRNESTQNCVVAEPEKMESSISSSSEEMSLSKCAEQF |
| | | CRLNEYNQLQLQAAHAQEQIRRMNENSHVQVPFQQLNQLAA |
| | | YPYAVWYYPQIMQYVPFPPFSDISNPTAHENYEKNNVMLQW |
| 29 | Human Alpha-S1-casein | MRPKLPLRYPERLQNPSESSEPIPLESREEYMNGMNRQRNILRE |
| | Mature Protein with | KQTDEIKDTRNESTQNCVVAEPEKMESSISSSSEEMSLSKCAEQ |
| | Methionine | FCRLNEYNQLQLQAAHAQEQIRRMNENSHVQVPFQQLNQLA |
| | | AYPYAVWYYPQIMQYVPFPPFSDISNPTAHENYEKNNVMLQ |
| | | W |
| 30 | Bovine Alpha-S2 Casein | MKFFIFTCLLAVALAKNTMEHVSSSEESIISQETYKQEKNMAI |
| | | NPSKENLCSTFCKEVVRNANEEEYSIGSSSEESAEVATEEVKIT |
| | | VDDKHYQKALNEINQFYQKFPQYLQYLYQGPIVLNPWDQVK |
| | | RNAVPITPTLNREQLSTSEENSKKTVDMESTEVFTKKTKLTEEE |
| | | KNRLNFLKKISQRYQKFALPQYLKTVYQHQKAMKPWIQPKTK |
| | | VIPYVRYL |
| 31 | Bovine Alpha-S2 Casein | KNTMEHVSSSEESIISQETYKQEKNMAINPSKENLCSTFCKEVV |
| | Mature protein | RNANEEEYSIGSSSEESAEVATEEVKITVDDKHYQKALNEINQ |
| | | FYQKFPQYLQYLYQGPIVLNPWDQVKRNAVPITPTLNREQLST |
| | | SEENSKKTVDMESTEVFTKKTKLTEEEKNRLNFLKKISQRYQK |
| | | FALPQYLKTVYQHQKAMKPWIQPKTKVIPYVRYL |
| 32 | Bovine Alpha-S2 Casein | MKNTMEHVSSSEESIISQETYKQEKNMAINPSKENLCSTFCKE |
| | Mature protein with | VVRNANEEEYSIGSSSEESAEVATEEVKITVDDKHYQKALNEI |
| | Methionine | NQFYQKFPQYLQYLYQGPIVLNPWDQVKRNAVPITPTLNREQ |
| | | LSTSEENSKKTVDMESTEVFTKKTKLTEEEKNRLNFLKKISQR |
| | | YQKFALPQYLKTVYQHQKAMKPWIQPKTKVIPYVRYL |
| 33 | Ovine Alpha-S2-casein | MKFFIFTCLLAVALAKHKMEHVSSSEEPINISQEIYKQEKNM |
| | | AIHPRKEKLCTTSCEEVVRNADEEEYSIRSSSEESAEVAPEEVKI |
| | | TVDDKHYQKALNEINQFYQKFPQYLQYLYQGPIVLNPWDQV |
| | | KRNAGPFTPTVNREQLSTSEENSKKTIDMESTEVFTKKTKLTE |
| | | EEKNRLNFLKKISQYYQKFAWPQYLKTVDQHQKAMKPWTQP |
| | | KTNAIPYVRYL |
| 34 | Ovine Alpha-S2-casein | KHKMEHVSSSEEPINISQEIYKQEKNMAIHPRKEKLCTTSCEEV |
| | Mature protein | VRNADEEEYSIRSSSEESAEVAPEEVKITVDDKHYQKALNEIN |
| | | QFYQKFPQYLQYLYQGPIVLNPWDQVKRNAGPFTPTVNREQL |
| | | STSEENSKKTIDMESTEVFTKKTKLTEEEKNRLNFLKKISQYYQ |
| | | KFAWPQYLKTVDQHQKAMKPWTQPKTNAIPYVRYL |
| 35 | Ovine Alpha-S2-casein | MKHKMEHVSSSEEPINISQEIYKQEKNMAIHPRKEKLCTTSCEE |
| | Mature protein with | VVRNADEEEYSIRSSSEESAEVAPEEVKITVDDKHYQKALNEI |
| | Methionine | NQFYQKFPQYLQYLYQGPIVLNPWDQVKRNAGPFTPTVNREQ |
| | | LSTSEENSKKTIDMESTEVFTKKTKLTEEEKNRLNFLKKISQYY |
| | | QKFAWPQYLKTVDQHQKAMKPWTQPKTNAIPYVRYL |
| 36 | Caprine Alpha-S2-casein | MKFFIFTCLLAVALAKHKMEHVSSSEEPINIFQEIYKQEKNM |
| | | AIHPRKEKLCTTSCEEVVRNANEEEYSIRSSSEESAEVAPEEIKI |
| | | TVDDKHYQKALNEINQFYQKFPQYLQYPYQGPIVLNPWDQV |
| | | KRNAGPFTPTVNREQLSTSEENSKKTIDMESTEVFTKKTKLTE |
| | | EEKNRLNFLKKISQYYQKFAWPQYLKTVDQHQKAMKPWTQP |
| | | KTNAIPYVRYL |
| 37 | Caprine Alpha-S2-casein | KHKMEHVSSSEEPINIFQEIYKQEKNMAIHPRKEKLCTTSCEEV |
| | Mature Protein | VRNANEEEYSIRSSSEESAEVAPEEIKITVDDKHYQKALNEINQ |
| | | FYQKFPQYLQYPYQGPIVLNPWDQVKRNAGPFTPTVNREQLS |
| | | TSEENSKKTIDMESTEVFTKKTKLTEEEKNRLNFLKKISQYYQ |
| | | KFAWPQYLKTVDQHQKAMKPWTQPKTNAIPYVRYL |
| 38 | Caprine Alpha-S2-casein | MKHKMEHVSSSEEPINIFQEIYKQEKNMAIHPRKEKLCTTSCEE |
| | Mature Protein with | VVRNANEEEYSIRSSSEESAEVAPEEIKITVDDKHYQKALNEIN |
| | Methionine | QFYQKFPQYLQYPYQGPIVLNPWDQVKRNAGPFTPTVNREQL |
| | | STSEENSKKTIDMESTEVFTKKTKLTEEEKNRLNFLKKISQYYQ |
| | | KFAWPQYLKTVDQHQKAMKPWTQPKTNAIPYVRYL |
| 39 | Buffalo Alpha-S2-casein | MKFFIFTCLLAVALAKHTMEHVSSSEESIISQETYKQEKNMAI |
| | | HPSKENLCSTFCKEVIRNANEEEYSIGSSSEESAEVATEEVKITV |
| | | DDKHYQKALNEINQFYQKFPQYLQYLYQGPIVLNPWDQVKR |
| | | NAVPITPTLNREQLSTSEENSKKTVDMESTEVFTKKTKLTEED |
| | | KNRLNFLKKISQHYQKFAWPQYLKTVYQYQKAMKPWTQPKT |
| | | NVIPYVRYL |
| 40 | Buffalo Alpha-S2-casein | KHTMEHVSSSEESIISQETYKQEKNMAIHPSKENLCSTFCKEVI |
| | Mature Protein | RNANEEEYSIGSSSEESAEVATEEVKITVDDKHYQKALNEINQ |
| | | FYQKFPQYLQYLYQGPIVLNPWDQVKRNAVPITPTLNREQLST |
| | | SEENSKKTVDMESTEVFTKKTKLTEEDKNRLNFLKKISQHYQ |
| | | KFAWPQYLKTVYQYQKAMKPWTQPKTNVIPYVRYL |
| 41 | Buffalo Alpha-S2-casein | MKHTMEHVSSSEESIISQETYKQEKNMAIHPSKENLCSTFCKE |
| | Mature Protein with | VIRNANEEEYSIGSSSEESAEVATEEVKITVDDKHYQKALNEIN |
| | Methionine | QFYQKFPQYLQYLYQGPIVLNPWDQVKRNAVPITPTLNREQLS |
| | | TSEENSKKTVDMESTEVFTKKTKLTEEDKNRLNFLKKISQHYQ |
| | | KFAWPQYLKTVYQYQKAMKPWTQPKTNVIPYVRYL |
| 42 | Equine Alpha-S2-casein | MKFFIFTCLLAVALAKHNMEHRSSSEDSVNISQEKFKQEKYV |
| | | VIPTSKESICSTSCEEATRNINEMESAKFPTEREEKEVEEKHHLK |
| | | QLNKINQFYEKLNFLQYLQALRQPRIVLTPWDQTKTGDSPFIPI |
| | | VNTEQLFTSEEIPKKTVDMESTEVVTEKTELTEEEKNYLKLLY |
| | | YEKFTLPQYFKIVRQHQTTMDPRSHRKTNSYQIIPVLRYF |
| 43 | Equine Alpha-S2-casein | KHNMEHRSSSEDSVNISQEKFKQEKYVVIPTSKESICSTSCEEA |
| | Mature Protein | TRNINEMESAKFPTEREEKEVEEKHHLKQLNKINQFYEKLNFL |
| | | QYLQALRQPRIVLTPWDQTKTGDSPFIPIVNTEQLFTSEEIPKKT |
| | | VDMESTEVVTEKTELTEEEKNYLKLLYYEKFTLPQYFKIVRQH |
| | | QTTMDPRSHRKTNSYQIIPVLRYF |
| 44 | Equine Alpha-S2-casein | MKHNMEHRSSSEDSVNISQEKFKQEKYVVIPTSKESICSTSCEE |
| | Mature Protein with | ATRNINEMESAKFPTEREEKEVEEKHHLKQLNKINQFYEKLNF |
| | Methionine | LQYLQALRQPRIVLTPWDQTKTGDSPFIPIVNTEQLFTSEEIPKK |
| | | TVDMESTEVVTEKTELTEEEKNYLKLLYYEKFTLPQYFKIVRQ |
| | | HQTTMDPRSHRKTNSYQIIPVLRYF |
| 45 | Camel Alpha-S2-casein | MKFFIFTCLLAVVLAKHEMDQGSSSEESINVSQQKFKQVKKV |
| | | AIHPSKEDICSTFCEEAVRNIKEVESAEVPTENKISQFYQKWKF |
| | | LQYLQALHQGQIVMNPWDQGKTRAYPFIPTVNTEQLSISEEST |
| | | EVPTEESTEVFTKKTELTEEEKDHQKFLNKIYQYYQTFLWPEY |
| | | LKTVYQYQKTMTPWNHIKRYF |
| 46 | Camel Alpha-S2-casein | KHEMDQGSSSEESINVSQQKFKQVKKVAIHPSKEDICSTFCEE |
| | Mature Protein | AVRNIKEVESAEVPTENKISQFYQKWKFLQYLQALHQGQIVM |
| | | NPWDQGKTRAYPFIPTVNTEQLSISEESTEVPTEESTEVFTKKT |
| | | ELTEEEKDHQKFLNKIYQYYQTFLWPEYLKTVYQYQKTMTP |
| | | WNHIKRYF |
| 47 | Camel Alpha-S2-casein | MKHEMDQGSSSEESINVSQQKFKQVKKVAIHPSKEDICSTFCE |
| | Mature Protein with | EAVRNIKEVESAEVPTENKISQFYQKWKFLQYLQALHQGQIV |
| | Methionine | MNPWDQGKTRAYPFIPTVNTEQLSISEESTEVPTEESTEVFTKK |
| | | TELTEEEKDHQKFLNKIYQYYQTFLWPEYLKTVYQYQKTMTP |
| | | WNHIKRYF |
| 48 | Bovine Kappa Casein | MMKSFFLVVTILALTLPFLGAQEQNQEQPIRCEKDERFFSDK |
| | | IAKYIPIQYVLSRYPSYGLNYYQQKPVALINNQFLPYPYYAKP |
| | | AAVRSPAQILQWQVLSNTVPAKSCQAQPTTMARHPHPHLSFM |
| | | AIPPKKNQDKTEIPTINTIASGEPTSTPTTEAVESTVATLEDSPE |
| | | VIESPPEINTVQVTSTAV |
| 49 | Bovine Kappa Casein Mature | QEQNQEQPIRCEKDERFFSDKIAKYIPIQYVLSRYPSYGLNYYQ |
| | protein | QKPVALINNQFLPYPYYAKPAAVRSPAQILQWQVLSNTVPAK |
| | | SCQAQPTTMARHPHPHLSFMAIPPKKNQDKTEIPTINTIASGEP |
| | | TSTPTTEAVESTVATLEDSPEVIESPPEINTVQVTSTAV |
| 50 | Bovine Kappa Casein Mature | MQEQNQEQPIRCEKDERFFSDKIAKYIPIQYVLSRYPSYGLNYY |
| | protein with Methionine | QQKPVALINNQFLPYPYYAKPAAVRSPAQILQWQVLSNTVPA |
| | | KSCQAQPTTMARHPHPHLSFMAIPPKKNQDKTEIPTINTIASGE |
| | | PTSTPTTEAVESTVATLEDSPEVIESPPEINTVQVTSTAV |
| 51 | Bovine (B. taurus) kappa | QEQNQEQPIRCEKDERFFSDKIAKYIPIQYVLSRYPSYGLNYYQ |
| | casein(B) aa 1-153 (A154- | QKPVALINNQFLPYPYYAKPAAVRSPAQILQWQVLSNTVPAK |
| | 169) | SCQAQPTTMARHPHPHLSFMAIPPKKNQDKTEIPTINTIASGEP |
| | | TSTPTIEAVESTVATLEASPEVI |
| 52 | Bovine (B. taurus) kappa | MQEQNQEQPIRCEKDERFFSDKIAKYIPIQYVLSRYPSYGLNYY |
| | casein(B) aa 1-153 (A154- | QQKPVALINNQFLPYPYYAKPAAVRSPAQILQWQVLSNTVPA |
| | 169) with Methionine | KSCQAQPTTMARHPHPHLSFMAIPPKKNQDKTEIPTINTIASGE |
| | | PTSTPTIEAVESTVATLEASPEVI |
| 53 | Ovine Kappa casein | MMKSFFLVVTILALTLPFLGAQEQNQEQRICCEKDERFFDDK |
| | | IAKYIPIQYVLSRYPSYGLNYYQQRPVALINNQFLPYPYYAKPV |
| | | AVRSPAQTLQWQVLPNAVPAKSCQDQPTAMARHPHPHLSFM |
| | | AIPPKKDQDKTEIPAINTIASAEPTVHSTPTTEAVVNAVDNPEA |
| | | SSESIASAPETNTAQVTSTEV |
| 54 | Ovine Kappa casein Mature | QEQNQEQRICCEKDERFFDDKIAKYIPIQYVLSRYPSYGLNYY |
| | protein | QQRPVALINNQFLPYPYYAKPVAVRSPAQTLQWQVLPNAVPA |
| | | KSCQDQPTAMARHPHPHLSFMAIPPKKDQDKTEIPAINTIASAE |
| | | PTVHSTPTTEAVVNAVDNPEASSESIASAPETNTAQVTSTEV |
| 55 | Ovine Kappa casein Mature | MQEQNQEQRICCEKDERFFDDKIAKYIPIQYVLSRYPSYGLNY |
| | protein with Methionine | YQQRPVALINNOFLPYPYYAKPVAVRSPAQTLQWQVLPNAVP |
| | | AKSCQDQPTAMARHPHPHLSFMAIPPKKDQDKTEIPAINTIAS |
| | | AEPTVHSTPTTEAVVNAVDNPEASSESIASAPETNTAQVTSTEV |
| 56 | Caprine Kappa casein | MMKSFFLVVTILALTLPFLGAQEQNQEQPICCEKDERFFDDK |
| | | IAKYIPIQYVLSRYPSYGLNYYQQRPVALINNQFLPYPYYAKPV |
| | | AVRSPAQTLQWQVLPNTVPAKSCQDQPTTLARHPHPHLSFMA |
| | | IPPKKDQDKTEVPAINTIASAEPTVHSTPTTEAIVNTVDNPEASS |
| | | ESIASASETNTAQVTSTEV |
| 57 | Caprine Kappa casein Mature | QEQNQEQPICCEKDERFFDDKIAKYIPIQYVLSRYPSYGLNYYQ |
| | Protein | QRPVALINNQFLPYPYYAKPVAVRSPAQTLQWQVLPNTVPAK |
| | | SCQDQPTTLARHPHPHLSFMAIPPKKDQDKTEVPAINTIASAEP |
| | | TVHSTPTTEAIVNTVDNPEASSESIASASETNTAQVTSTEV |
| 58 | Caprine Kappa casein Mature | MQEQNQEQPICCEKDERFFDDKIAKYIPIQYVLSRYPSYGLNY |
| | Protein with Methionine | YQQRPVALINNQFLPYPYYAKPVAVRSPAQTLQWQVLPNTVP |
| | | AKSCQDQPTTLARHPHPHLSFMAIPPKKDQDKTEVPAINTIAS |
| | | AEPTVHSTPTTEAIVNTVDNPEASSESIASASETNTAQVTSTEV |
| 59 | Buffalo Kappa Casein | MMKSFFLVVTILALTLPFLGAQEQNQEQPIRCEKEERFFNDK |
| | | IAKYIPIQYVLSRYPSYGLNYYQQKPVALINNQFLPYPYYAKP |
| | | AAVRSPAQILQWQVLPNTVPAKSCQAQPTTMTRHPHPHLSFM |
| | | AIPPKKNQDKTEIPTINTIVSVEPTSTPTTEAIENTVATLEASSEV |
| | | IESVPETNTAQVTSTVV |
| 60 | Buffalo Kappa Casein | QEQNQEQPIRCEKEERFFNDKIAKYIPIQYVLSRYPSYGLNYYQ |
| | Mature Protein | QKPVALINNQFLPYPYYAKPAAVRSPAQILQWQVLPNTVPAK |
| | | SCQAQPTTMTRHPHPHLSFMAIPPKKNQDKTEIPTINTIVSVEP |
| | | TSTPTTEAIENTVATLEASSEVIESVPETNTAQVTSTVV |
| 61 | Buffalo Kappa Casein | MQEQNQEQPIRCEKEERFFNDKIAKYIPIQYVLSRYPSYGLNY |
| | Mature Protein with | YQQKPVALINNQFLPYPYYAKPAAVRSPAQILQWQVLPNTVP |
| | Methionine | AKSCQAQPTTMTRHPHPHLSFMAIPPKKNQDKTEIPTINTIVSV |
| | | EPTSTPTTEAIENTVATLEASSEVIESVPETNTAQVTSTVV |
| 62 | Equine Kappa Casein | MKSFFLVVNILALTLPFLGAEVQNQEQPTCHKNDERFFDLKT |
| | | VKYIPIYYVLNSSPRYEPIYYQHRLALLINNQHMPYQYYARPA |
| | | AVRPHVQIPQWQVLPNIYPSTVVRHPCPHPSFIAIPPKKLQEITV |
| | | IPKINTIATVEPTPIPTPEPTVNNAVIPDASSEFIIASTPETTTVPV |
| | | TSPVVQKL |
| 63 | Equine Kappa Casein Mature | EVQNQEQPTCHKNDERFFDLKTVKYIPIYYVLNSSPRYEPIYY |
| | Protein | QHRLALLINNQHMPYQYYARPAAVRPHVQIPQWQVLPNIYPS |
| | | TVVRHPCPHPSFIAIPPKKLQEITVIPKINTIATVEPTPIPTPEPTV |
| | | NNAVIPDASSEFIIASTPETTTVPVTSPVVQKL |
| 64 | Equine Kappa Casein Mature | MEVQNQEQPTCHKNDERFFDLKTVKYIPIYYVLNSSPRYEPIY |
| | Protein with Methionine | YQHRLALLINNQHMPYQYYARPAAVRPHVQIPQWQVLPNIYP |
| | | STVVRHPCPHPSFIAIPPKKLQEITVIPKINTIATVEPTPIPTPEPT |
| | | VNNAVIPDASSEFIIASTPETTTVPVTSPVVQKL |
| 65 | Camel Kappa casein | MKSFFLVVTILALTLPFLGAEVQNQEQPTCFEKVERLLNEKT |
| | | VKYFPIQFVQSRYPSYGINYYQHRLAVPINNQFIPYPNYAKPV |
| | | AIRLHAQIPQCQALPNIDPPTVERRPRPRPSFIAIPPKKTQDKTV |
| | | NPAINTVATVEPPVIPTAEPAVNTVVIAEASSEFITTSTPETTTV |
| | | QITSTEI |
| 66 | Camel Kappa casein Mature | EVQNQEQPTCFEKVERLLNEKTVKYFPIQFVQSRYPSYGINYY |
| | Protein | QHRLAVPINNQFIPYPNYAKPVAIRLHAQIPQCQALPNIDPPTV |
| | | ERRPRPRPSFIAIPPKKTQDKTVNPAINTVATVEPPVIPTAEPAV |
| | | NTVVIAEASSEFITTSTPETTTVQITSTEI |
| 67 | Camel Kappa casein Mature | MEVQNQEQPTCFEKVERLLNEKTVKYFPIQFVQSRYPSYGINY |
| | Protein with Methionine | YQHRLAVPINNQFIPYPNYAKPVAIRLHAQIPQCQALPNIDPPT |
| | | VERRPRPRPSFIAIPPKKTQDKTVNPAINTVATVEPPVIPTAEPA |
| | | VNTVVIAEASSEFITTSTPETTTVQITSTEI |
| 68 | Human Kappa Casein | MKSFLLVVNALALTLPFLAVEVQNQKQPACHENDERPFYQK |
| | | TAPYVPMYYVPNSYPYYGTNLYQRRPAIAINNPYVPRTYYAN |
| | | PAVVRPHAQIPQRQYLPNSHPPTVVRRPNLHPSFIAIPPKKIQD |
| | | KIIIPTINTIATVEPTPAPATEPTVDSVVTPEAFSESIITSTPETTTV |
| | | AVTPPTA |
| 69 | Human Kappa Casein Mature | EVQNQKQPACHENDERPFYQKTAPYVPMYYVPNSYPYYGTN |
| | Protein | LYQRRPAIAINNPYVPRTYYANPAVVRPHAQIPQRQYLPNSHP |
| | | PTVVRRPNLHPSFIAIPPKKIQDKIIIPTINTIATVEPTPAPATEPT |
| | | VDSVVTPEAFSESIITSTPETTTVAVTPPTA |
| 70 | Human Kappa Casein Mature | MEVQNQKQPACHENDERPFYQKTAPYVPMYYVPNSYPYYGT |
| | Protein with Methionine | NLYQRRPAIAINNPYVPRTYYANPAVVRPHAQIPQRQYLPNSH |
| | | PPTVVRRPNLHPSFIAIPPKKIQDKIIIPTINTIATVEPTPAPATEPT |
| | | VDSVVTPEAFSESIITSTPETTTVAVTPPTA |
| 71 | Bovine Beta Casein | MKVLILACLVALALARELEELNVPGEIVESLSSSEESITRINKK |
| | | IEKFQSEEQQQTEDELQDKIHPFAQTQSLVYPFPGPIPNSLPQNI |
| | | PPLTQTPVVVPPFLQPEVMGVSKVKEAMAPKHKEMPFPKYPV |
| | | EPFTESQSLTLTDVENLHLPLPLLQSWMHQPHQPLPPTVMFPP |
| | | QSVLSLSQSKVLPVPQKAVPYPQRDMPIQAFLLYQEPVLGPVR |
| | | GPFPIIV |
| 72 | Bovine Beta Casein Mature | RELEELNVPGEIVESLSSSEESITRINKKIEKFQSEEQQQTEDEL |
| | Protein | QDKIHPFAQTQSLVYPFPGPIPNSLPQNIPPLTQTPVVVPPFLQP |
| | | EVMGVSKVKEAMAPKHKEMPFPKYPVEPFTESQSLTLTDVEN |
| | | LHLPLPLLQSWMHQPHQPLPPTVMFPPQSVLSLSQSKVLPVPQ |
| | | KAVPYPQRDMPIQAFLLYQEPVLGPVRGPFPIIV |
| 73 | Bovine Beta Casein Mature | MRELEELNVPGEIVESLSSSEESITRINKKIEKFQSEEQQQTEDE |
| | Protein with Methionine | LQDKIHPFAQTQSLVYPFPGPIPNSLPQNIPPLTQTPVVVPPFLQ |
| | | PEVMGVSKVKEAMAPKHKEMPFPKYPVEPFTESQSLTLTDVE |
| | | NLHLPLPLLQSWMHQPHQPLPPTVMFPPQSVLSLSQSKVLPVP |
| | | QKAVPYPQRDMPIQAFLLYQEPVLGPVRGPFPIIV |
| 74 | Ovine (O. aries) beta-casein | REQEELNVVGETVESLSSSEESITHINKKIEKFQSEEQQQTEDEL |
| | Mature Protein | QDKIHPFAQAQSLVYPFTGPIPNSLPQNILPLTQTPVVVPPFLQP |
| | | EIMGVPKVKETMVPKHKEMPFPKYPVEPFTESQSLTLTDVEKL |
| | | HLPLPLVQSWMHQPPQPLPPTVMFPPQSVLSLSQPKVLPVPQK |
| | | AVPQRDMPIQAFLLYQEPVLGPVRGPFPILV |
| 75 | Ovine (O. aries) beta-casein | MREQEELNVVGETVESLSSSEESITHINKKIEKFQSEEQQQTED |
| | Mature Protein with | ELQDKIHPFAQAQSLVYPFTGPIPNSLPQNILPLTQTPVVVPPFL |
| | Methionine | QPEIMGVPKVKETMVPKHKEMPFPKYPVEPFTESQSLTLTDVE |
| | | KLHLPLPLVQSWMHQPPQPLPPTVMFPPQSVLSLSQPKVLPVP |
| | | QKAVPQRDMPIQAFLLYQEPVLGPVRGPFPILV |
| 76 | Caprine (C. hircus) beta- | REQEELNVVGETVESLSSSEESITHINKKIEKFQSEEQQQTEDEL |
| | casein Mature Protein | QDKIHPFAQAQSLVYPFTGPIPNSLPQNILPLTQTPVVVPPFLQP |
| | | EIMGVPKVKETMVPKHKEMPFPKYPVEPFTESQSLTLTDVEKL |
| | | HLPLPLVQSWMHQPPQPLSPTVMFPPQSVLSLSQPKVLPVPQK |
| | | AVPQRDMPIQAFLLYQEPVLGPVRGPFPILV |
| 77 | Caprine (C. hircus) beta- | MREQEELNVVGETVESLSSSEESITHINKKIEKFQSEEQQQTED |
| | casein Mature Protein with | ELQDKIHPFAQAQSLVYPFTGPIPNSLPQNILPLTQTPVVVPPFL |
| | Methionine | QPEIMGVPKVKETMVPKHKEMPFPKYPVEPFTESQSLTLTDVE |
| | | KLHLPLPLVQSWMHQPPQPLSPTVMFPPQSVLSLSQPKVLPVP |
| | | QKAVPQRDMPIQAFLLYQEPVLGPVRGPFPILV |
| 78 | Buffalo (B. bubalis) beta- | RELEELNVPGEIVESLSSSEESITHINKKIEKFQSEEQQQMEDEL |
| | casein Mature Protein | QDKIHPFAQTQSLVYPFPGPIPKSLPQNIPPLTQTPVVVPPFLQP |
| | | EIMGVSKVKEAMAPKHKEMPFPKYPVEPFTESQSLTLTDVENL |
| | | HLPLPLLQSWMHQPPQPLPPTVMFPPQSVLSLSQSKVLPVPQK |
| | | AVPYPQRDMPIQAFLLYQEPVLGPVRGPFPIIV |
| 79 | Buffalo (B. bubalis) beta- | MRELEELNVPGEIVESLSSSEESITHINKKIEKFQSEEQQQMEDE |
| | casein Mature Protein with | LQDKIHPFAQTQSLVYPFPGPIPKSLPQNIPPLTQTPVVVPPFLQ |
| | Methionine | PEIMGVSKVKEAMAPKHKEMPFPKYPVEPFTESQSLTLTDVEN |
| | | LHLPLPLLQSWMHQPPQPLPPTVMFPPQSVLSLSQSKVLPVPQ |
| | | KAVPYPQRDMPIQAFLLYQEPVLGPVRGPFPIIV |
| 80 | Equine (E. caballus) beta- | REKEELNVSSETVESLSSNEPDSSSEESITHINKEKLQKFKHEGQ |
| | casein Mature Protein | QQREVERQDKISRFVQPQPVVYPYAEPVPYAVVPQSILPLAQP |
| | | PILPFLQPEIMEVSQAKETILPKRKVMPFLKSPIVPFSERQILNPT |
| | | NGENLRLPVHLIQPFMHQVPQSLLQTLMLPSQPVLSPPQSKVA |
| | | PFPQPVVPYPQRDTPVQAFLLYQDPRLGPTGELDPATQPIVAV |
| | | HNPVIV |
| 81 | Equine (E. caballus) beta- | MREKEELNVSSETVESLSSNEPDSSSEESITHINKEKLQKFKHE |
| | casein Mature Protein with | GQQQREVERQDKISRFVQPQPVVYPYAEPVPYAVVPQSILPLA |
| | Methionine | QPPILPFLQPEIMEVSQAKETILPKRKVMPFLKSPIVPFSERQILN |
| | | PTNGENLRLPVHLIQPFMHQVPQSLLQTLMLPSQPVLSPPQSK |
| | | VAPFPQPVVPYPQRDTPVQAFLLYQDPRLGPTGELDPATQPIV |
| | | AVHNPVIV |
| 82 | Camel (C. dromedarius) | REKEEFKTAGEALESISSSEESITHINKQKIEKFKIEEQQQTEDE |
| | beta-casein Mature Protein | QQDKIYTFPQPQSLVYSHTEPIPYPILPQNFLPPLQPAVMVPFLQ |
| | | PKVMDVPKTKETIIPKRKEMPLLQSPVVPFTESQSLTLTDLENL |
| | | HLPLPLLQSLMYQIPQPVPQTPMIPPQSLLSLSQFKVLPVPQQM |
| | | VPYPQRAMPVQAVLPFQEPVPDPVRGLHPVPQPLVPVIA |
| 83 | Camel (C. dromedarius) | MREKEEFKTAGEALESISSSEESITHINKQKIEKFKIEEQQQTED |
| | beta-casein Mature Protein | EQQDKIYTFPQPQSLVYSHTEPIPYPILPQNFLPPLQPAVMVPFL |
| | with Methionine | QPKVMDVPKTKETIIPKRKEMPLLQSPVVPFTESQSLTLTDLEN |
| | | LHLPLPLLQSLMYQIPQPVPQTPMIPPQSLLSLSQFKVLPVPQQ |
| | | MVPYPQRAMPVQAVLPFQEPVPDPVRGLHPVPQPLVPVIA |
| 84 | Human (H. sapiens) beta- | RETIESLSSSEESITEYKQKVEKVKHEDQQQGEDEHQDKIYPSF |
| | casein Mature Protein | QPQPLIYPFVEPIPYGFLPQNILPLAQPAVVLPVPQPEIMEVPKA |
| | | KDTVYTKGRVMPVLKSPTIPFFDPQIPKLTDLENLHLPLPLLQP |
| | | LMQQVPQPIPQTLALPPQPLWSVPQPKVLPIPQQVVPYPQRAV |
| | | PVQALLLNQELLLNPTHQIYPVTQPLAPVHNPISV |
| 85 | Human (H. sapiens) beta- | MRETIESLSSSEESITEYKQKVEKVKHEDQQQGEDEHQDKIYP |
| | casein Mature Protein with | SFQPQPLIYPFVEPIPYGFLPQNILPLAQPAVVLPVPQPEIMEVP |
| | Methionine | KAKDTVYTKGRVMPVLKSPTIPFFDPQIPKLTDLENLHLPLPLL |
| | | QPLMQQVPQPIPQTLALPPQPLWSVPQPKVLPIPQQVVPYPQR |
| | | AVPVQALLLNQELLLNPTHQIYPVTQPLAPVHNPISV |
| 86 | Bovine Alpha-S1 Casein | MEAESISSSEEIVPNSVEQKHIQKEDVPSERYLGYLEQLLRLKK |
| | (B. Taurus) aa 60-199 | YKVPQLEIVPNSAEERLHSMKEGIHAQQKEPMIGVNQELAYFY |
| | | PELFRQFYQLDAYPSGAWYYVPLGTQYTDAPSFSDIPNPIGSE |
| | | NSEKTTMPLW |

### Embodiments

Embodiment 1: A hybrid micelle composition, comprising an alpha casein protein and a kappa casein protein, wherein at least one of the alpha casein protein and the kappa casein protein are recombinantly produced; wherein one or more casein proteins are from different mammalian species; and wherein the alpha casein protein and the kappa casein protein are associated in micellar form.

Embodiment 2: The hybrid micelle composition of embodiment 1, wherein the alpha casein protein and the kappa casein protein are from a different mammalian species.

Embodiment 3: The hybrid micelle of embodiment 1 or embodiment 2, wherein the alpha casein protein comprises two or more alpha casein proteins.

Embodiment 4: The hybrid micelle composition of embodiment 1 or embodiment 2, wherein the alpha casein protein comprises two or more alpha casein proteins from a different mammalian species.

Embodiment 5: The hybrid micelle composition of embodiment 1 or embodiment 2, wherein the alpha casein protein comprises two or more alpha casein proteins from the same mammalian species.

Embodiment 6: The hybrid micelle composition of embodiment 1 or embodiment 2, wherein the kappa casein protein comprises two or more kappa casein proteins.

Embodiment 7: The hybrid micelle composition of embodiment 1 or embodiment 2, wherein the kappa casein protein comprises two or more kappa casein proteins from a different mammalian species.

Embodiment 8: The hybrid micelle composition of embodiment 1 or embodiment 2, wherein the kappa casein protein comprises two or more kappa casein proteins from the same mammalian species.

Embodiment 9: The hybrid micelle composition according to any of embodiments 1-8, wherein the alpha casein protein is a bovine alpha casein protein.

Embodiment 10: The hybrid micelle composition according to any of embodiments 1-8, wherein the alpha casein protein comprises an amino acid sequence of SEQ ID NO. 1-11, 18-20, 30-32 or 39-41, or an amino acid sequence with at least 90% sequence identity to any one of SEQ ID NOs. 1-11, 18-20, 30-32 or 39-41.

Embodiment 11: The hybrid micelle composition of embodiment 9 or embodiment 10, wherein the kappa casein protein is a kappa casein protein selected from the group consisting of ovine, caprine, equine or camel.

Embodiment 12: The hybrid micelle composition of embodiment 9 or embodiment 10, wherein the kappa casein protein comprises an amino acid sequence selected from the group consisting of SEQ ID NO. 53-70, or an amino acid sequence with at least 90% sequence identity to SEQ ID NO. 53-70.

Embodiment 13: The hybrid micelle composition according to any of embodiments 1-8, wherein the alpha casein protein is an ovine alpha casein protein.

Embodiment 14: The hybrid micelle composition according to any of embodiments 1-8, wherein the alpha casein protein comprises an amino acid sequence of SEQ ID NO. 12-14, SEQ ID NO. 33-35 or an amino acid sequence with 90% sequence identity to SEQ ID NOs12-14 or 33-35.

Embodiment 15: The hybrid micelle composition of embodiment 13 or embodiment 14, wherein the kappa casein protein is a kappa casein protein selected from the group consisting of bovine, caprine equine or camel.

Embodiment 16: The hybrid micelle composition of embodiment 13 or embodiment 14, wherein the kappa casein protein comprises an amino acid sequence selected from the group consisting of SEQ ID NO. 48-52 or 56-70, or an amino acid sequence with at least 90% sequence identity to SEQ ID NO. 48-52 or 56-70.

Embodiment 17: The hybrid micelle composition according to any of embodiments 1-8, wherein the alpha casein protein is a caprine alpha casein protein.

Embodiment 18: The hybrid micelle composition according to any of embodiments 1-8, wherein the alpha casein protein comprises an amino acid sequence of SEQ ID NO. 15-17, 36-38, or an amino acid sequence with at least 90% sequence identity to SEQ ID NOs. 15-17, 36-38.

Embodiment 19: The hybrid micelle composition of embodiment 17 or embodiment 18, wherein the kappa casein protein is a kappa casein protein selected from the group consisting of ovine, bovine, equine or camel.

Embodiment 20: The hybrid micelle composition of embodiment 17 or embodiment 18, wherein the kappa casein protein comprises an amino acid sequence selected from the group consisting of SEQ ID NO. 48-55, 59-70 or an amino acid sequence with at least 90% sequence identity to SEQ ID NO. 48-55 or 59-70.

Embodiment 21: The hybrid micelle composition according to any of embodiments 1-20, wherein the kappa casein protein is a recombinant protein.

Embodiment 22: The hybrid micelle composition according to any of embodiments 1-21, wherein the alpha casein protein is a recombinant protein.

Embodiment 23: The hybrid micelle composition according to any of embodiments 1-22, wherein the micellar form does not include a beta casein protein or a derivative thereof.

Embodiment 24: The hybrid micelle composition according to any of embodiments 1-22, wherein the hybrid micelle comprises a beta casein protein.

Embodiment 25: The hybrid micelle composition of embodiment 24, wherein the beta casein protein is from the same species as the alpha casein protein of the hybrid micelle composition.

Embodiment 26: The hybrid micelle composition of embodiment 24, wherein the beta casein protein is from the same species as the kappa casein protein of the hybrid micelle composition.

Embodiment 27: The hybrid micelle composition of embodiment 24, wherein the beta casein protein is from a species that is different from the alpha casein protein and the kappa casein protein of the hybrid micelle composition.

Embodiment 28: The hybrid micelle composition of embodiment 24, wherein the beta casein protein is selected from a full-length beta casein protein, a gamma casein protein, or an alternate truncation of beta casein protein.

Embodiment 29: The hybrid micelle composition of embodiment 24, wherein the beta casein protein is a recombinant protein.

Embodiment 30: A hybrid micelle composition, comprising an alpha casein protein and a kappa casein protein, wherein the kappa casein protein comprises a deletion in the amino acid sequence as compared to a native kappa casein protein sequence; and wherein the alpha casein protein and the kappa casein protein are associated in micellar form.

Embodiment 31: The hybrid micelle composition of embodiment 30, wherein the kappa casein protein is a recombinant protein.

Embodiment 32: The hybrid micelle composition of embodiment 31, wherein the deletion comprises a C-terminal truncation of the kappa casein protein amino acid sequence.

Embodiment 33: The hybrid micelle composition of embodiment 32, wherein the kappa casein protein lacks between 1 and 49 C-terminal amino acids.

Embodiment 34: The hybrid micelle composition of embodiment 32, where the kappa casein protein comprises a bovine amino acid sequence truncated after amino acid 142, 146 or 153 of SEQ ID NO. 51.

Embodiment 35: The hybrid micelle composition according to any of embodiments 26-30, wherein the kappa casein protein is from a species selected from the group consisting of bovine, ovine or caprine.

Embodiment 36: The hybrid micelle composition according to any of embodiments 30-35, wherein the alpha casein protein is a recombinant protein.

Embodiment 37: The hybrid micelle composition according to any of embodiments 30-36, wherein the alpha casein protein is alpha-S1 or alpha-S2.

Embodiment 38: The hybrid micelle composition according to any of embodiments 30-37, further comprising a second kappa casein protein, wherein the second kappa casein protein does not comprise the deletion in the amino acid sequence as compared to the native kappa casein protein sequence.

Embodiment 39: A hybrid micelle composition, comprising an alpha casein protein and a kappa casein protein, wherein the alpha casein protein comprises a deletion in the amino acid sequence as compared to the native alpha casein protein sequence; and wherein the alpha casein protein and the kappa casein protein are associated in micellar form.

Embodiment 40: The hybrid micelle composition of embodiment 39, wherein the alpha casein protein is a recombinant protein.

Embodiment 41: The hybrid micelle composition of embodiment 39 or embodiment 40, wherein the alpha casein protein is an alpha-S1 casein protein.

Embodiment 42: The hybrid micelle composition of embodiment 41, wherein the deletion comprises a N-terminal truncation of the alpha-S1 casein protein amino acid sequence.

Embodiment 43: The hybrid micelle composition of embodiment 42, wherein the alpha-S1-casein protein lacks between 1 and 59 N-terminal amino acids.

Embodiment 44: The hybrid micelle composition of embodiment 42 where the alpha-S1-casein protein comprises a bovine amino acid sequence starting at amino acid 23, 24, 25 or 26 of SEQ ID NO. 3.

Embodiment 45: The hybrid micelle composition according to any of embodiments 39-44, wherein the kappa casein protein comprises a C-terminal truncation of the kappa casein protein amino acid sequence.

Embodiment 46: The hybrid micelle composition of embodiment 45, wherein the kappa casein protein lacks between 1 and 27 C-terminal amino acids.

Embodiment 47: The hybrid micelle composition of embodiment 45, where the kappa casein protein comprises a bovine amino acid sequence truncated after amino acid 142, 146 or 153 of SEQ ID NO. 51.

Embodiment 48: The hybrid micelle composition according to any of embodiments 40-47, wherein the alpha casein protein and/or the kappa casein protein is from a species selected from the group consisting of bovine, ovine or caprine.

Embodiment 49: The hybrid micelle composition according to any of embodiments 40-48, wherein the kappa casein protein is a recombinant protein.

Embodiment 50: The hybrid micelle composition according to any of embodiments 40-49, further comprising a second alpha casein protein, wherein the second alpha casein protein does not comprise the deletion in the amino acid sequence as compared to the native alpha casein protein sequence.

Embodiment 51: The hybrid micelle composition according to any of embodiments 30-50 further comprising a beta casein protein.

Embodiment 52: The hybrid micelle composition of embodiment 51, wherein the beta casein protein is a selected from a full-length beta casein protein, a gamma casein protein, or an alternate truncation of beta casein protein.

Embodiment 53: The hybrid micelle composition according to any of embodiments 30-50, wherein the micellar form does not include a beta casein protein.

Embodiment 54: The hybrid micelle composition according to any of embodiments 1-53 wherein the alpha casein protein is not phosphorylated or is substantially reduced in phosphorylation as compared to native alpha casein protein.

Embodiment 55: The hybrid micelle composition according to any of embodiments 1-53, wherein the alpha casein protein comprises a phosphorylation pattern that differs from native alpha casein protein.

Embodiment 56: The hybrid micelle composition according to any of embodiments 1-55, wherein the kappa casein protein is not glycosylated or is substantially reduced in glycosylation as compared to native kappa casein protein.

Embodiment 57: The hybrid micelle composition according to any of embodiments 1-55, wherein the kappa casein protein comprises a glycosylation pattern that differs from native kappa casein protein.

Embodiment 58: The hybrid micelle composition according to any of embodiments 1-57, wherein the ratio of alpha casein protein to kappa casein protein is between about 1:1 and about 10:1 or between about 1:1 and 5:1.

Embodiment 59: A colloid comprising the hybrid micelle composition according to any of embodiments 1-58.

Embodiment 60: A dairy-like product comprising the hybrid micelle composition according to any of embodiments 1-58.

Embodiment 61: The dairy-like product of embodiment 60, wherein the dairy-like product is selected from the group consisting of milk, cream, ice cream and yogurt.

Embodiment 62: The dairy-like product of embodiment 60, wherein the dairy-like product is a cheese analogue.

Embodiment 63: The dairy-like product of embodiment 60, wherein the dairy-like product is a coagulated colloid of the hybrid micelle composition.

Embodiment 64: The dairy-like product of embodiment 63, wherein the coagulated colloid is cheese curd.

Embodiment 65: The dairy-like product of embodiment 63, wherein the coagulated colloid is cheese.

Embodiment 66: The dairy-like product of embodiment 64, wherein the cheese is a soft cheese, a hard cheese or an aged cheese.

Embodiment 67: The cheese composition of embodiment 64, wherein the cheese is selected from the group consisting of pasta-filata like cheese, paneer, cream cheese, and cottage cheese.

Embodiment 68: The cheese composition of embodiment 64, wherein the cheese is selected from the group consisting of mozzarella, cheddar, swiss, brie, camembert, feta, halloumi, gouda, edam, cheddar, manchego, swiss, Colby, muenster, blue cheese and parmesan.

Embodiment 69: The dairy-like product of embodiment 63, wherein the coagulated colloid is yogurt.

Embodiment 70: The dairy-like product according to any of embodiments 60-69, wherein the dairy-like product does not include any animal-sourced dairy protein.

Embodiment 71: The dairy-like product according to any of embodiments 60-69, wherein the dairy-like product does not include any dairy-related protein other than caseins.

Embodiment 72: The dairy-like product according to any of embodiments 60-69, wherein the dairy-like product comprises at least one additional protein other than caseins.

Embodiment 73: The dairy-like product of embodiment 72, wherein the at least one additional protein is a dairy-related protein other than caseins.

Embodiment 74: The dairy-like product of embodiment 73, wherein the dairy-related protein is a whey protein.

Embodiment 75: The dairy-like product of embodiment 72, wherein the at least one additional protein is a plant protein.

Embodiment 76: A method for producing a hybrid micelle composition, comprising: providing an alpha casein protein and a kappa casein protein, wherein at least one of the alpha casein protein and the kappa casein protein is a recombinant protein, and wherein (a) the alpha casein protein and the kappa casein protein are from a different mammalian species, and/or (b) the kappa casein protein comprises a deletion in the amino acid sequence as compared to a native kappa casein protein sequence; and/or (c) the alpha casein protein comprises a deletion in the amino acid sequence as compared to a native alpha casein protein sequence; and combining the alpha casein protein and a kappa casein protein and at least one salt under conditions wherein alpha casein protein and a kappa casein protein form a micellar form in a liquid colloid.

Embodiment 77: The method of embodiment 76, wherein the salt is selected from the group consisting of a calcium salt, a citrate salt, a phosphate salt and any combination thereof.

Embodiment 78: The method of embodiment 76 or embodiment 77, wherein the micellar form further comprises a beta casein protein.

Embodiment 79: The method of embodiment 76 or embodiment 77, wherein the micellar form lacks a beta casein protein.

Embodiment 80: The method according to any of embodiments 76-78, wherein the beta casein protein comprises a full-length beta casein protein, a gamma casein protein or an alternate truncation of beta casein protein.

Embodiment 81: The method according to any of embodiments 76-80, further comprising subjecting the liquid colloid to a first condition to form coagulates.

Embodiment 82: The method of embodiment 81, wherein the first condition is the addition of acid or acidification of the liquid colloid with a microorganism.

Embodiment 83: The method of embodiment 81 or embodiment 82, wherein the method further comprises subjecting the coagulates to a hot water treatment and optionally stretching, to form a filata-type cheese.

Embodiment 84: The method of embodiment 81, wherein the method further comprises subjecting the coagulates to a renneting agent to form a rennetted curd.

Embodiment 85: The method of embodiment 84, wherein the renneting agent is a microbially-derived chymosin enzyme.

Embodiment 86: The method of embodiment 84 or embodiment 85, wherein the method further comprises aging and maturing the rennetted curd to form a cheese-like food product.

Embodiment 87: The method of embodiment 84 or embodiment 85, wherein the method further comprises subjecting the rennetted curd to a hot water treatment and optionally stretching, to form a filata-type cheese food product.

Embodiment 88: The method according to any of embodiments 76-87, wherein the recombinantly produced alpha casein protein and/or kappa casein protein are produced from a microbial host cell.

Embodiment 89: The method of embodiment 88, wherein the microbial host cell is selected from the group consisting of a bacteria, a yeast, or a fungus.

Embodiment 90: The method of embodiment 88, wherein the microbial host cell is a bacteria selected from the group consisting of Lactococci sp., Lactococcus lactis, Bacillus subtilis, Bacillus amyloliquefaciens, Bacillus licheniformis and Bacillus megaterium, Brevibacillus choshinensis, Mycobacterium smegmatis, Rhodococcus erythropolis and Corynebacterium glutamicum, Lactobacilli sp., Lactobacillus fermentum, Lactobacillus casei, Lactobacillus acidophilus, Lactobacillus plantarum, Synechocystis sp. 6803 and E. coli.

Embodiment 91: The method according to any of embodiments 76-80, further comprising drying the liquid colloid to produce a micellar casein containing protein powder.

Embodiment 92: The method of embodiment 87, wherein the drying comprises spray-drying or freeze-drying.

Embodiment 93: The method according to any of embodiments 76-80, further comprising subjecting the liquid colloid to salt or acid precipitation to produce a caseinate-like protein powder.

Embodiment 94: A protein powder comprising the hybrid micelle composition according to any of embodiments 1-58.

Embodiment 95: A protein powder produced by the method according to any of embodiments 76-80 or 88-93.

Embodiment 96: The protein powder of embodiment 94 or 95 where the powder is a spray-dried or freeze-dried powder.

Embodiment 97: A dairy-like product comprising the protein powder according to any of embodiments 94-96.

Embodiment 98: The dairy-like product of embodiment 97, wherein the dairy-like product is selected from the group consisting of milk, cream, ice-cream, yogurt, mozzarella cheese analogue, curd and cheese.

Embodiment 99: A micelle-like composition, comprising kappa casein protein, wherein the kappa casein is associated in a micellar-like form; and wherein the micelle-like composition lacks alpha casein and beta casein protein.

Embodiment 100: The micelle-like composition of embodiment 99, wherein the kappa casein protein comprises two or more kappa casein proteins from different mammalian species.

Embodiment 101: The micelle-like composition of embodiment 99, wherein the kappa casein protein comprises two or more kappa casein proteins and at least one of the kappa casein proteins comprises a deletion in the amino acid sequence as compared to a native kappa casein protein sequence.

Embodiment 102: The micelle-like composition of embodiment 100 or embodiment 101, at least one of the kappa casein proteins is recombinantly produced.

Embodiment 103: The micelle-like composition of embodiment 99, wherein the kappa casein protein is a kappa casein protein selected from the group consisting of ovine, caprine, equine or camel.

Embodiment 104: The micelle-like composition according to any of embodiments 100-102, wherein at least one of the kappa casein proteins is a kappa casein protein selected from the group consisting of ovine, caprine, equine or camel.

Embodiment 105: The micelle-like composition according to any of embodiments 100-102, wherein at least one of the kappa casein proteins comprises an amino acid sequence selected from the group consisting of SEQ ID NO. 48-70, or an amino acid sequence with at least 90% sequence identity to SEQ ID NO. 48-70.

Embodiment 106: The micelle-like composition according to any of embodiments 102-105, wherein the recombinantly produced kappa casein protein is produced from a microbial host cell.

Embodiment 107: The micelle-like composition of embodiment 106, wherein the microbial host cell is selected from the group consisting of a bacteria, a yeast, or a fungus.

Embodiment 108: The micelle-like composition of embodiment 106, wherein the microbial host cell is a bacteria selected from the group consisting of Lactococci sp., Lactococcus lactis, Bacillus subtilis, Bacillus amyloliquefaciens, Bacillus licheniformis and Bacillus megaterium, Brevibacillus choshinensis, Mycobacterium smegmatis, Rhodococcus erythropolis and Corynebacterium glutamicum, Lactobacilli sp., Lactobacillus fermentum, Lactobacillus casei, Lactobacillus acidophilus, Lactobacillus plantarum, Synechocystis sp. 6803 and E. coli.

Embodiment 109: A protein powder comprising the micelle-like composition according to any of embodiments 99-108.

Embodiment 110: A colloid comprising the micelle-like composition according to any of embodiments 99-108.

Embodiment 111: A dairy-like product comprising the micelle-like composition according to any of embodiments 99-108.

Embodiment 112: The dairy-like product of embodiment 111, wherein the dairy-like product is selected from the group consisting of milk, cream, ice cream and yogurt.

Embodiment 113: The dairy-like product of embodiment 111, wherein the dairy-like product is a cheese analogue.

### EXAMPLES

The following illustrative examples are representative of embodiments of the compositions and methods described herein and are not meant to be limiting in any way.

### Example 1: Expression of Casein Proteins in Lactococcus lactis Via Nisin-Inducible System (NICE)

### Constructs Design, Cloning and Transformation

Bovine kappa casein (variant B) and bovine alpha-S1-casein (variant C) protein coding sequences (without the native signal peptide) were codon-optimized for expression in Lactococcus lactis and a synthetic operon was constructed for co-expression and secretion of the two proteins under a nisin-inducible promoter. Signal peptide sequence from natively secreting lactococcal protein Usp45 was used to drive protein secretion. A synthetic operon was then cloned into an E. coli custom vector via restriction digest compatible sites and confirmed via Sanger sequencing, from which it was subcloned into nisin-inducible pNZ8149 vector via restriction digestion and ligation. The vector was transformed into compatible L. lactis strain NZ3900 via electroporation and completely defined media (CDM) supplemented with lactose was used for selection. Positive clones were confirmed via colony PCR and 3 positive clones were taken forward for the protein expression induction and analysis.

### Protein Expression and Analysis

Individual colonies were grown at 30° C. in liquid culture and protein production was induced with nisin for 2.5 hours (control samples left uninduced). Cells were then harvested by centrifugation and TCA-precipitated supernatants and lysed cell pellets were analysed by Coomassie gel staining (SDS-PAGE) and chemiluminescence (Western Blot against kappa casein and alpha-S1-casein, LSBio primary antibodies). Kappa casein expression in L. lactis was detected in the tested transformants by Coomassie stained protein gel and western blot.

### Example 2: Expression in L. lactis Via pH-Inducible System

Similar to the constructions above, casein protein constructions were created for alpha, beta and kappa casein replacing the nisin promoter with the P170 promoter, a pH/lactate inducible promoter for L. lactis. Each of these constructs contained a secretion signal peptide.

Both alpha-S1 and kappa casein were detected in L. lactis upon secretion on western blot. Protein product accumulated intracellularly for alpha-S1-casein. Alpha-S1-casein secreted poorly, whereas kappa casein showed near-complete secretion of protein produced.

### Example 3: Expression in B. subtilis

### Constructs Design, Cloning and Transformation

Bovine alpha-S1-casein (variant C) protein coding sequence (without the native signal peptide) His-tagged C-terminally was codon-optimized for expression in Bacillus subtilis. Constructs were created with and without the codon-optimized signal peptide of amyQ, alpha-amylase Bacillus amyloliquefaciens which has been reported for the efficient secretion of recombinant proteins. Constructs were cloned through E. coli via Gibson cloning into transformation and expression IPTG-inducible vector pHT01 and confirmed via Sanger sequencing. pHT01 is an E. coli/B. subtilis shuttle vector that provides ampicillin resistance to E. coli and chloramphenicol resistance to B. subtilis. Positive clones were further transformed into chemically competent B. subtilis WB800N. Positive clones were confirmed via colony PCR and 3 positive clones were taken forward for the protein expression induction and analysis.

### Protein Expression and Analysis

Individual colonies were grown at 37° C. in liquid culture and protein production was induced with IPTG for 1 hour, 2 hours and 6 hours (control samples were left uninduced). Cells were then harvested by centrifugation, and TCA-precipitated supernatants and lysed cell pellets were analysed by Coomassie gel staining (SDS-PAGE) and chemiluminescence (Western Blot against His tag and alpha-S1-casein).

Western blotting showed expression of the alpha-S1-casein in B. subtilis.

### Example 4: Expression in E. coli

### Constructs Design, Cloning and Transformation

Bovine alpha-S1-casein (variant C) protein coding sequence (without the native signal peptide) codon-optimized for Escherichia coli was cloned into IPTG-inducible commercially available pET vectors. Cloning was performed via Gibson reaction of DNA fragments and vector in such a way that only the protein coding sequence was left within the open reading frame. Gibson reactions were transformed into competent cells and confirmed by Sanger sequencing. Vectors were then transformed into chemically competent E. coli BL21(DE3) cells, or their derivatives (e.g. BL21-pLysS), and several single colonies were screened for expression.

### Protein Expression, Analysis and Purification

Individual colonies were grown at 37 C in liquid culture, and protein production was induced with IPTG for 4 hours. Cells were then harvested by centrifugation, and lysed cell pellets were analysed by Coomassie gel staining (SDS-PAGE) and chemiluminescence (Western Blot against alpha-S1-casein). For protein purification, the insoluble fraction was removed by centrifugation and the soluble fraction was then precipitated with ammonium sulfate at room temperature and pelleted by centrifugation. The pellet was resuspended in urea, followed by dialysis against disodium phosphate. The insoluble proteins were removed by centrifugation, and the remaining contaminants were removed by precipitation with ethanol and ammonium acetate followed by centrifugation. The resulting alpha-S1-casein solution was concentrated using a centrifugal filtration unit and then dialyzed against di sodium phosphate. Purified product was analysed on a Coomassie stained gel similarly to explained above.

Alpha-S1-casein was expressed intracellularly in E. coli, successfully detected on Coomassie stained protein gel and purified.

### Example 5: Expression of Recombinant α Casein, κ Casein, Truncated Recombinant a Casein and Truncated Recombinant κ Casein

### Constructs Design, Cloning and Transformation

Alpha-S1-casein, kappa casein, N-terminally truncated alpha-S1-casein and C-terminally truncated kappa casein protein coding sequence (without the native signal peptide, with or without an N-terminal His-tag or His-SUMO-tag) were each codon-optimized for Escherichia coli and were cloned individually into IPTG-inducible commercially available pET vectors. Cloning was performed via Gibson reaction of DNA fragments (IDT) and vector in such a way that only the protein coding sequence was left within the open reading frame. Gibson reactions were transformed into competent cells and confirmed by Sanger sequencing. Vectors were then transformed into chemically competent E. coli BL21(DE3) cells, or their derivatives (e.g. BL21(DE3) pLysS, Rosetta (DE3)), and several single colonies were screened for expression. This way, the following expression vectors for producing alpha-S1-casein variants were created: pET-alpha-S1-casein(bovine), pET-6×His-alpha-S1-casein(bovine), pET-6×His-SUMO-alpha-S1-casein(bovine), pET-F24-alpha-S1-casein(bovine) (N-terminal 23 amino acids truncation), pET-6×His-F24-alpha-S1-casein(bovine), pET-6×His-SUMO-F24-alpha-S1-casein(bovine), pET-alpha-S1-casein(ovine), pET-6×His-alpha-S1-casein(ovine), pET-6×His-SUMO-alpha-S1-casein(ovine), pET-alpha-S1-casein(caprine), pET-6×His-alpha-S1-casein(caprine), pET-6×His-SUMO-alpha-S1-casein(caprine).

The following expression vectors for producing kappa casein variants were created: pET-kappa casein(bovine), pET-6×His-kappa casein(bovine), pET-6×His-SUMO-kappa casein(bovine), pET-kappa casein-delta154(bovine) (C-terminal 16 amino acids truncation), pET-6×His-kappa casein-delta154 (bovine), pET-6×His-SUMO-kappa casein-delta154 (bovine), pET-kappa casein(ovine), pET-6×His-kappa casein(ovine), pET-6×His-SUMO-kappa casein(ovine), pET-kappa casein(caprine), pET-6×His-kappa casein(caprine), pET-6×His-SUMO-kappa casein(caprine).

### Protein Induction and Expression

An individual colony for each of the transformants was inoculated into TB medium containing 0.2% (v/v) glycerol and 100 μg/ml ampicillin or 50 μg/ml kanamycin. Cells were grown at 37 C overnight in a shaking incubator. This overnight culture was used to inoculate 1 L of fresh TB medium containing 0.2% (v/v) glycerol and 100 μg/ml ampicillin or 50 μg/ml kanamycin, and cells were grown until OD600 reached ˜0.5-0.6, at which point isopropyl-thiogalactopyranoside (IPTG) was added to 0.5 mM. After four hours of incubation at 37 C, the cells were harvested by centrifugation and frozen at −80 C. Protein purification and analysis Frozen cell pellets were thawed on ice and resuspended in lysis buffer (40 mM Tris, pH 8, 0.3 M NaCl) supplemented with 2 mM Pefabloc, 0.1% (v/v) Triton X-100. The suspensions were lysed using a sonicator. The resulting total crude lysate was centrifuged at 10,000×g for 10 min at 4 C to separate soluble and insoluble material. The soluble material was applied to equilibrated immobilized Ni-NTA Agarose resin, incubated for 1 hour on rotator at 4 C, and transferred to a gravity column to collect the beads. The resin beads were washed four times with 5-bed volume of wash buffer (40 mM Tris, pH 8, 0.3 M NaCl, 20 mM Imidazole) to remove non-specifically bound proteins. His-tagged proteins were eluted in 2-bed volume of elution buffer (40 mM Tris, pH 8, 0.3 M NaCl, 300 mM Imidazole). Following purification, protein samples were either dialyzed overnight in 10 mM K2HPO4 (if protein is not processed further) or in a buffer required for Ulp1 cleavage of SUMO tag. 6×His-SUMO-casein protein constructs were then used in proteolytic cleavage reaction with 6×His-Ulp1 at 4 C overnight to generate untagged casein variants. Proteolysed material was applied onto Ni-NTA Agarose resin in a ‘negative purification’, where the flow through and wash which contain the untagged casein variant were collected. Final untagged alpha-S1-casein variants, kappa casein variants and truncated kappa casein variants were dialyzed overnight in 10 mM K2HPO4. Cell lysates as well as the purified products were analysed on a Coomassie stained SDS-PAGE.

### Example 6: Hybrid Micelles from Caseins from Different Species and their Colloid Properties

Bovine alpha-casein purified from cow's milk (Sigma), hypophosphorylated (˜2-3 phosphates per molecule) version of bovine alpha-casein (Sigma) and unphosphorylated bovine alpha-S1-casein, recombinantly produced, were used in combination with a caprine kappa casein, recombinantly produced, to form hybrid species casein micelles. 10.4 mg/ml of alpha-casein was mixed with 3.6 mg/ml kappa casein, and micelles were induced using phosphate, citrate and calcium salts at the following final concentrations: phosphate 12.4 mM, citrate 6.15 mM, calcium 18.5 mM. The resulting colloids were evaluated using dynamic light scattering (DLS) for particle size measurement and absorbance (A450) for turbidity measurement.

For particle sizing measurement, samples were diluted to a concentration of 1.4 mg/mL of protein or less in filtered (220 nm) milliQ water. 50 μL samples were used for measurement and three replicates were measured at a 173° detection angle over the amount of time determined by the instrument using Zetasizer (Malvern). The data was analyzed using the Zetasizer's small peak analysis mode. For turbidity measurement, samples were diluted to a concentration of 0.7 mg/ml in filtered (220 nm) milliQ water and absorbance was measured at 450 nm in 1 ml cuvettes using Spectramax.

The resulting colloids were subjected to curd and cheesemaking, and the efficiency of cheesemaking was estimated (curd formation quality, stretch quality and yield). Samples were acidified by titrating 6.6% citric acid until the pH was between 5.0 and 5.2. Samples were then renneted at room temperature using 0.15% rennet solution at 1.36% of the final micellar colloid volume. Curds were spun @ 1,000×g for 1 minute, drained from liquid and submerged in a 65° C. water bath (duration dependent on curd size), stretched into mozzarella balls, placed on a wipe to remove excess moisture, and weighed.

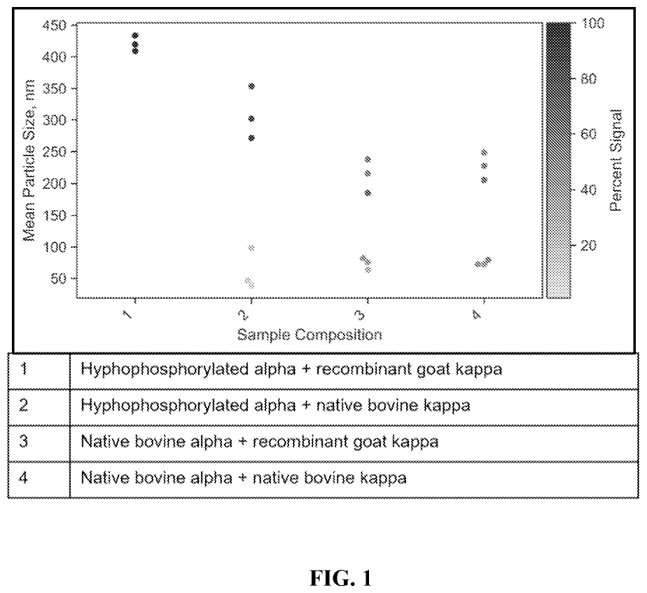

FIG. 1 shows that bovine kappa and goat kappa caseins are each able to form micelles in combination with bovine alpha-casein. The relative particle sizes and their distributions between the hybrid micelles and the all-bovine casein micelles was similar. FIG. 1 also shows that reduced phosphorylation on alpha-casein leads to larger micelle size, independently of whether the bovine or caprine kappa casein is used.

TABLE 2
A450 of colloids subjected to particle size analysis in FIG. 1

| | | Hyphophosphorylated |
|---|---|---|
| | Bovine Alpha | Bovine Alpha |
| Bovine Kappa | 3.2 | 4.4 |
| Recombinant Goat Kappa | 3.3 | 3.5 |

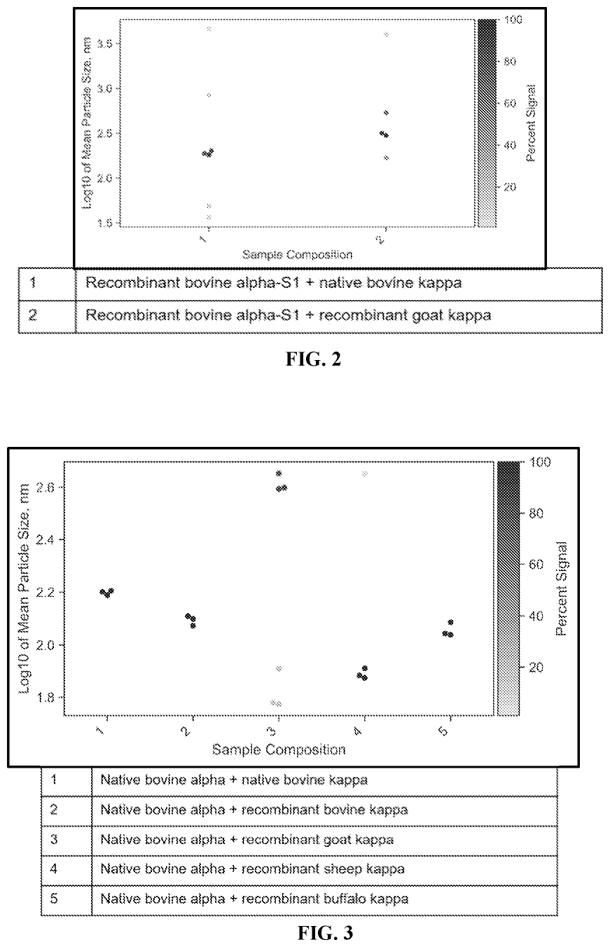

TABLE 3
A450 of colloids subjected to particle size analysis in FIG. 2

| | Dairy Kappa | Recombinant Goat Kappa |
|---|---|---|
| Unphosphorylated | 1.18 | 0.84 |
| Recombinant Bovine | | |
| Alpha-S1 | | |

All samples produced cohesive curds able to withstand the ‘tube inversion’ test, where curds stay complete when inverted under gravity, except for hypophosphorylated bovine alpha+recombinant goat kappa sample curd, which was weaker/partially shattered. All curds were able to melt and stretch without demonstrating brittleness and without snapping/breaking under stretch prematurely.

Table 4 shows that cheese formation occurs when hybrid micelles are formed using caprine (goat) kappa casein in combination with bovine alpha-casein, as well as micelles that contain both alpha and kappa casein from the same organism (bovine). Cheese yields are similar between the hybrid micelles and the all-bovine casein micelles. Table 4 shows that reduced phosphorylation on alpha-casein has only a slight effect on cheese yields (however not significantly, within 10% of the control value), independently of whether the bovine or caprine kappa casein is used.

TABLE 4
Mozzarella cheese yield (%, g of cheese per g of casein) for cheeses made
from colloids in Table 2

| | | Hyphophosphorylated |
|---|---|---|
| | Bovine Alpha | Bovine Alpha |
| Bovine Kappa | 187% | 175% |
| Recombinant Goat Kappa | 184% | 173% |

TABLE 5
Mozzarella cheese yield (%, g of cheese per g of casein) for
cheeses made from colloids in Table 3

| | Dairy Kappa | Recombinant Goat Kappa |
|---|---|---|
| Recombinant Bovine Alpha | 156% | 135% |

### Example 7: Hybrid Micelles from Bovine Alpha Casein Variants and Kappa Casein from Different Species, their Colloid Properties, and Cheesemaking

Native bovine alpha-casein purified from cow's milk (Sigma Aldrich) and recombinantly-produced bovine (cow) alpha-S1-casein (lacking PTMs), were used in combination with caprine (goat) kappa casein, ovine (sheep) kappa casein and buffalo kappa casein, recombinantly produced (lacking PTMs), to form hybrid species casein micelles. As a control for native system casein micelles, bovine alpha-casein purified from cow's milk (Sigma Aldrich) and recombinantly-produced bovine (cow) alpha-S1-casein (lacking PTMs) were used in combination with bovine kappa casein purified from cow's milk (Sigma Aldrich) and recombinantly-produced bovine (cow) kappa casein (lacking PTMs), 10.4 mg/ml of alpha-casein was mixed with 3.6 mg/ml kappa casein, and micelles were induced using 12.4 mM phosphate, 6.15 mM citrate, and 18.5 mM calcium. Micelles containing recombinantly produced proteins were induced using 20 mM phosphate, 10 mM citrate and 27 mM calcium. The resulting colloids were evaluated using dynamic light scattering (DLS) for particle size measurement and absorbance (A450) for turbidity measurement.

For particle sizing measurement, samples were diluted to a 1.4 mg/mL concentration of protein or less in filtered (220 nm) milliQ water. 50 ul samples were used for measurement, and three replicates were measured at a 173° detection angle over the amount of Zetasizer's small peak analysis mode. For turbidity measurements, samples were diluted to a concentration of 0.7 mg/ml in filtered (220 nm) milliQ water, and absorbance was measured at 450 nm in 1 ml cuvettes using Spectramax.

The resulting colloids were subjected to curd and cheesemaking, and the efficiency of cheesemaking was estimated (curd formation quality, stretch and melt quality, and yield). Samples were acidified by titrating 6.6% citric acid until the pH was between 5 and Samples were then renneted at room temperature using 0.15% rennet solution at 1.36% of the final micellar colloid volume. Curds were spun at 1,000×g for 1 minute, drained from liquid and submerged in a 70° C. water bath (duration dependent on curd size), stretched into mozzarella balls, placed on a wipe to remove excess moisture, and weighed.

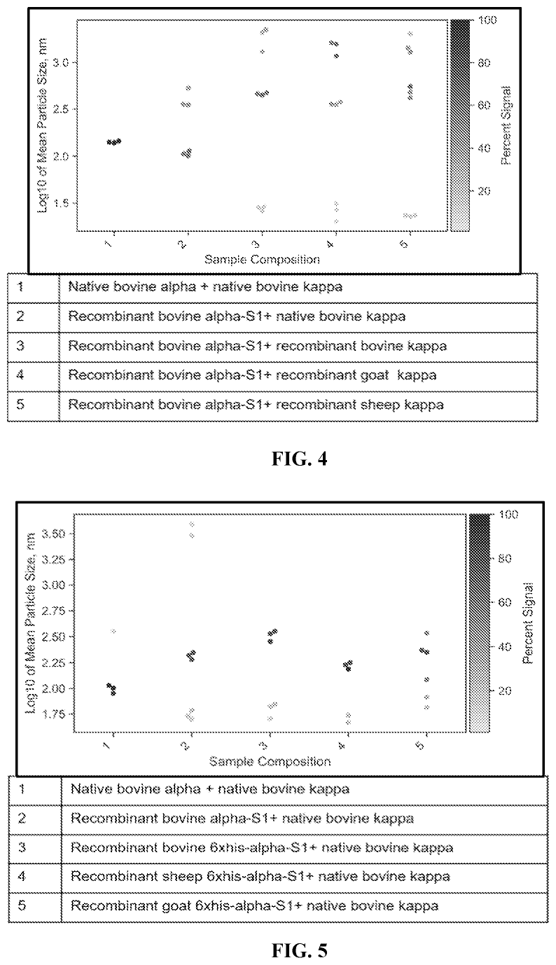

FIG. 3 shows that bovine kappa casein, both recombinant and native, recombinant caprine kappa, ovine kappa, and buffalo kappa caseins, lacking PTMs, were each able to form micelles with native bovine alpha-casein. Hybrid species micelles formed using recombinant buffalo kappa casein were the most similar in size to the native bovine micelles. Surprisingly, recombinant caprine and ovine kappa caseins, which differ only in 7 amino acids between one another, and both come from ruminant species, formed different size hybrid micelles with native bovine alpha-casein. As can be seen in FIG. 3, caprine kappa casein formed larger sized hybrid micelles (370-470 nm) and ovine kappa casein formed smaller sized hybrid micelles (˜80 nm). FIG. 4 shows that micelles formed using both alpha-S1 and kappa casein recombinantly produced, where alpha-S1-casein is lacking phosphorylation, had larger particle sizes (˜400 nm main micellar population) independently of whether the bovine, caprine, or ovine kappa casein was used. These micelles from recombinant caseins also formed a distinct smaller sub-micellar population in size range of 20-40 nm.

TABLE 6
A450 of colloids subjected to particle size analysis in FIG. 3 and FIG. 4

| | Bovine Alpha | Recombinant |
|---|---|---|
| A450 | (Native) | Bovine Alpha-s1 |
| Bovine Kappa (Native) | 6.38 | 0.66 |
| Recombinant Bovine Kappa | 3.76 | 1.52 |
| Recombinant Goat Kappa | 19.70 | 1.88 |
| Recombinant Sheep Kappa | 1 | 1.94 |
| Recombinant Buffalo Kappa | 2.78 | NA |

TABLE 7
Mozzarella cheese yield (%, g of cheese per g of casein), stretch and
melt for cheeses made from colloids in Table 3 or FIG. 3 and FIG. 4

| | | | | Recombinant Bovine | | |
|---|---|---|---|---|---|---|
| | Native Bovine Alpha | | | Alpha-s1 | | |
| | Stretch | Melt | Yield | Stretch | Melt | Yield |
| Native Bovine Kappa | + | +++ | 140% | + | +++ | 190% |
| Recombinant Bovine Kappa | +++ | +++ | 170% | +++ | +++ | 130% |
| Recombinant Goat Kappa | +++ | +++ | 170% | − | ++ | 140% |
| Recombinant Sheep Kappa | +++ | +++ | 160% | +++ | +++ | 145% |
| Recombinant Buffalo Kappa | +++ | +++ | 140% | na | na | na |

All micellar colloid samples produced cohesive curds able to withstand the tube inversion test, except for recombinantly-made native-like bovine kappa casein in combination with bovine alpha-S1-casein sample that produced weaker/partially shattered curd. Table 7 shows cheese formation from hybrid micelles samples having bovine alpha-casein in combination with kappa casein from different species (caprine, ovine, buffalo) and all-bovine micelles having alpha and kappa casein from the same species. All cheeses melted and stretched well in the all-bovine and hybrid micelles samples containing native bovine alpha-casein in combination with native or recombinant kappa caseins from bovine, caprine, ovine, or buffalo. Table 7 shows cheese made from the hybrid micelles having recombinant alpha-S1-casein, lacking phosphorylation, in combination with caprine (goat) kappa casein showed no stretch and significantly reduced melt. Surprisingly, the cheese made from hybrid micelles having recombinant bovine alpha-S1-casein, lacking phosphorylation, in combination with ovine (sheep) kappa casein showed the same extent of stretch and melt compared to cheese made from all-bovine (native-like) micelles having native phosphorylated bovine alpha-casein and native phosphorylated and glycosylated bovine kappa casein. Table 7 also shows that the yield of cheeses made from hybrid micelles was in the same range as those made from all-bovine micelles.

### Example 8: Hybrid Micelles Having Alpha-Casein from Different Species and Bovine Kappa-Casein, their Colloid Properties, and Cheesemaking

Native bovine alpha-casein purified from cow's milk (Sigma Aldrich), recombinantly-produced N-terminally 6×histidine-tagged alpha-s1-casein (lacking PTMs) from bovine (cow), ovine (sheep) and caprine (goat), and recombinantly-produced bovine (cow) alpha-s1-casein (lacking PTMs and 6×histidine tag), were used in combination with bovine (cow) kappa casein purified from cow's milk (Sigma Aldrich), to form hybrid species casein micelles. 10.4 mg/ml of alpha-casein was mixed with 3.6 mg/ml kappa casein, and micelles were induced using 12.4 mM phosphate, 6.15 mM citrate, and 18.5 mM calcium. Micelles containing recombinantly produced proteins were induced using 20 mM phosphate, mM citrate and 27 mM calcium. The resulting colloids were evaluated using dynamic light scattering (DLS) for particle size measurement and absorbance (A450) for turbidity measurement.

For particle sizing measurement, samples were diluted to 1.4 mg/mL concentration of protein or less in filtered (220 nm) milliQ water. 50 ul samples were used for measurement, and three replicates were measured at a 173° detection angle over the amount of Zetasizer's small peak analysis mode. For turbidity measurements, samples were diluted to a concentration of 0.7 mg/ml in filtered (220 nm) milliQ water, and absorbance was measured at 450 nm in 1 ml cuvettes using Spectramax.

The resulting colloids were subjected to curd and cheesemaking, and the efficiency of cheesemaking was estimated (curd formation quality, stretch and melt quality, and yield). Samples were acidified by titrating 6.6% citric acid until the pH was between 5 and 6.4. Samples were then renneted at room temperature using 0.15% rennet solution at 1.36% of the final micellar colloid volume. Curds were spun at 1,000×g for 1 minute, drained from liquid and submerged in a 70° C. water bath (duration dependent on curd size), stretched into mozzarella balls, placed on a wipe to remove excess moisture, and weighed.

FIG. 5 shows bovine alpha-casein, both recombinant alpha-S1 and native, caprine alpha-S1-casein and ovine alpha-S1-casein were each able to form micelles with bovine kappa casein (native/dairy). The hybrid micelles having ovine 6×his-alpha-S1-casein and bovine kappa casein formed somewhat larger particles (280-420 nm main micellar population) compared to other hybrid and all-bovine recombinant alpha-S1-casein micelles combinations (150-260 nm main micellar population). Surprisingly, the all-bovine micelles having recombinant bovine 6×his-alpha-S1-casein and bovine kappa casein had relatively smaller particle sizes (150-180 nm) compared to all-bovine micelles having recombinant bovine alpha-S1-casein (without the 6×his tag) and bovine kappa casein (190-260 nm) suggesting the N-terminal tag on alpha-s1-casein likely affects its interaction with caseins in micelle formation, but not in a destructive/inhibitory way. Interestingly, table 8 turbidity data suggests that for N-terminally 6his-tagged alpha-S1-caseins micelle formation efficiency was better for hybrid micelles (caprine and ovine alpha-S1-casein with bovine kappa casein) than for native system micelles (bovine alpha-S1 and kappa casein).

TABLE 8
Average A450 of colloids subjected to particle size analysis in FIG. 5

| A450 | Bovine Kappa (Native/Dairy) |
|---|---|
| Bovine Alpha (Native/Dairy) | 0.5 |
| Recombinant Bovine Alpha-s1 | 0.27 |
| Recombinant Bovine 6xhis-alpha-s1 | 0.13 |
| Recombinant Caprine | 0.95 |
| 6xhis-alpha-s1 | |
| Recombinant Ovine | 3.6 |
| 6xhis-alpha-s1 | |

TABLE 9
Average Mozzarella cheese yield (%, g of cheese
per g of casein), stretch and melt for
cheeses made from colloids in Table 5 and FIG. 5

| | Bovine Kappa (Native/Dairy) | | |
|---|---|---|---|
| | Stretch | Melt | Yield |
| Bovine Alpha (Native/Dairy) | +++ | +++ | 185% |
| Recombinant Bovine Alpha-s1 | +++ | +++ | 155% |
| Recombinant Bovine 6xhis-alpha-s1 | + | + | 145% |
| Recombinant Caprine | + | + | 165% |
| 6xhis-alpha-s1 | | | |
| Recombinant Ovine | + | + | 150% |
| 6xhis-alpha-s1 | | | |

The micelles having recombinantly-produced 6×histidine-tagged alpha-S1-casein from all species combined with bovine kappa casein required higher pH (>6.2) during acidification to prevent protein precipitation. When this pH of renneting was kept above the micelle precipitation pH, all samples produced cohesive curds able to withstand the tube inversion test.

All cheeses melted and stretched to some extent. Table 9 shows cheese from hybrid micelles containing ovine or caprine 6×his-alpha-S1-casein and bovine kappa casein stretched and melted to the same extent as the cheese from all-bovine micelles containing bovine 6×his alpha-S1-casein and bovine kappa casein. However, cheeses from all 6×his-tagged alpha-S1-casein samples (hybrid or native micelles) did not stretch and melt to the same extent as the cheese from alpha-casein, native or recombinant, lacking the N-terminal 6×his tag. The relative difference in the acidification, curd properties, and cheese stretch and melt likely arises from the presence of N-terminal 6×his tag on alpha-s1-casein.

### Example 9: Effect of Reducing Kappa Casein to Alpha-Casein Ratio on Hybrid Micelles Having Bovine Alpha-Casein and Kappa Casein from Different Species, their Colloid Properties, and Cheesemaking

Recombinantly-produced bovine alpha-s1-casein (lacking PTMs), was used in combination with recombinantly-produced ovine kappa casein and caprine kappa casein (lacking PTMs) to form hybrid species casein micelles at 2.8% total casein concentration. 21 mg/ml, 22.4 mg/ml, and 23.4 mg/L of alpha-casein was mixed with 7 mg/ml, 5.6 mg/ml, and 4.6 mg/ml of kappa casein respectively to bring the alpha-casein to kappa casein ratios to 3:1, 4:1 and 5:1. Micelles were induced using 20 mM phosphate, 12 mM citrate, and 26 mM calcium. The resulting colloids were evaluated using dynamic light scattering (DLS) for particle size measurement and absorbance (A450) for turbidity measurement.

For particle sizing measurement, samples were diluted to 1.4 mg/mL concentration of protein or less in filtered (220 nm) milliQ water. 50 ul samples were used for measurement, and three replicates were measured at a 173° detection angle over the amount of Zetasizer's small peak analysis mode. For turbidity measurements, samples were diluted to a concentration of 0.7 mg/ml in filtered (220 nm) milliQ water, and absorbance was measured at 450 nm in 1 ml cuvettes using Spectramax.

The resulting colloids were subjected to curd and cheesemaking, and the efficiency of cheesemaking was estimated (curd formation quality, stretch and melt quality, and yield). Samples were acidified by titrating 6.6% citric acid until the pH was between 5 and Samples were then renneted at room temperature using 0.15% rennet solution at 1.36% of the final micellar colloid volume. Curds were spun at 1,000×g for 1 minute, drained from liquid and submerged in a 70° C. water bath (duration dependent on curd size), stretched into mozzarella balls, placed on a wipe to remove excess moisture, and weighed.

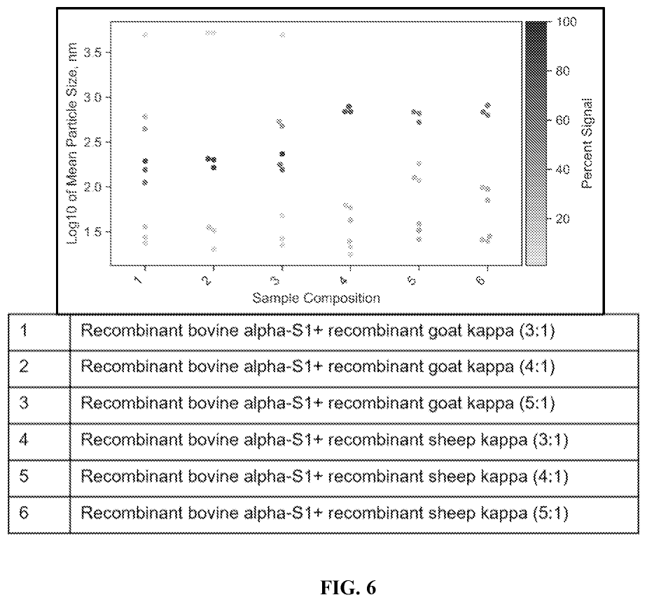

FIG. 6 shows recombinant bovine alpha-S1-casein, lacking PTMs, was able to form hybrid micelles with caprine and ovine kappa casein in all the tested ratios of alpha-S1-casein to kappa casein. Hybrid micelles and colloids made from recombinant bovine alpha-S1-casein and recombinant ovine kappa casein formed particles whose main population was in 500-900 nm range, and sub-micelle population in 20-100 nm range, with no larger aggregates observed. Hybrid micelles and colloids made from recombinant bovine alpha-S1-casein and recombinant caprine kappa casein formed particles whose main population was in 100-300 nm range, and sub-micelle population in 20-100 nm range, while also some larger micelle populations (600-800 nm) and aggregates (>1000 nm) were present. Table 10 shows the turbidity of hybrid micelle colloids.

TABLE 10
Average A450 of colloids subjected to particle size analysis in FIG. 6

| | Turbidity of recombinant bovine alpha- | | | | | Turbidity of recombinant bovine alpha-S1- | | | | |
|---|---|---|---|---|---|---|---|---|---|---|
| | S1-casein and recombinant caprine | | | | | casein and recombinant ovine kappa casein | | | | |
| | kappa casein hybrid micelles | | | | | hybrid micelles | | | | |
| | Rep 1 | Rep 2 | Rep 3 | Avg. | Stdev | Rep 1 | Rep 2 | Rep 3 | Avg. | Stdev |
| Alpha- | 2.24 | 2.48 | 2.3 | 2.34 | 0.12 | 3.06 | 3.02 | 2.42 | 1.68 | 0.36 |
| S1: | | | | | | | | | | |
| kappa, | | | | | | | | | | |
| 3:1 | | | | | | | | | | |
| Alpha- | 1.44 | 1.62 | 1.38 | 1.48 | 0.12 | 3.12 | 2.46 | 2.7 | 2.76 | 0.33 |
| S1: | | | | | | | | | | |
| kappa, | | | | | | | | | | |
| 4:1 | | | | | | | | | | |
| Alpha- | 1.4 | 1.34 | 1.38 | 1.37 | 0.03 | 2.9 | 2.42 | 2.36 | 2.56 | 0.30 |
| S1: | | | | | | | | | | |
| kappa, | | | | | | | | | | |
| 5:1 | | | | | | | | | | |

Hybrid micelles formed from bovine alpha-S1-casein and ovine kappa casein and their colloid gave cohesive curds upon renneting able to withstand the tube inversion test at all tested alpha-S1-casein to kappa casein ratios. Similar findings were observed for curds from bovine alpha-S1-casein and caprine kappa casein hybrid micelles, except in the 5:1 alpha-S1-casein to kappa casein ratios where the curds were softer and slid down the tube during the tube inversion test.

TABLE 11
Mozzarella cheese yield (%, g of cheese per g of casein), for cheeses made from
colloids in Table 10 and FIG. 6

| | Cheese yield from micelles having | | | | | Cheese yield from micelles having | | | | |
|---|---|---|---|---|---|---|---|---|---|---|
| | recombinant bovine alpha-S1-casein + | | | | | recombinant bovine alpha-S1-casein + | | | | |
| | recombinant caprine kappa casein | | | | | recombinant ovine kappa casein | | | | |
| | Rep 1 | Rep 2 | Rep 3 | Avg. | Stdev | Rep 1 | Rep 2 | Rep 3 | Avg. | Stdev |
| Alpha: kappa, 3:1 | 188% | 205% | 190% | 194% | 9% | 207% | 202% | 219% | 209% | 9% |
| Alpha: kappa, 4:1 | 195% | 192% | 192% | 193% | 2% | 214% | 227% | 225% | 222% | 7% |
| Alpha: kappa, 5:1 | 214% | 190% | 208% | 204% | 12% | 235% | 228% | 230% | 231% | 4% |

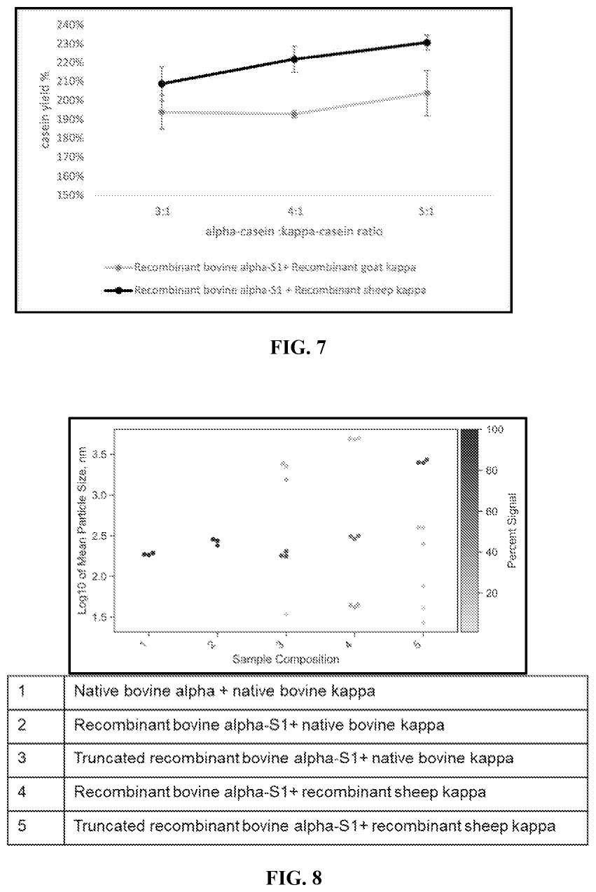

The cheeses made from bovine alpha-S1-casein and ovine kappa casein hybrid micelles stretched and melted very well irrespective of the alpha-S1-casein to kappa casein ratios within the range tested. On the contrary, the cheeses made from bovine alpha-S1-casein and caprine kappa casein hybrid micelles stretched poorly. Table 8 and FIG. 7 shows that cheese yields were increased and driven by the amount (proportion) of alpha-S1-casein used, while the total casein concentration was fixed in the experiment across all casein ratios. This discovery allows us to control and improve the cheese yield by controlling for the amount (proportion) of alpha-S1-casein used in recombinant hybrid micelles.

### Example 10: Hybrid Micelles from Truncated Bovine a Casein and κ Casein Variants, their Colloid Properties, and Cheesemaking

Recombinantly-produced bovine (cow) alpha-S1-casein (lacking PTMs), and recombinantly-produced bovine (cow) alpha-S1-casein (lacking PTMs) with 23 amino acid residues truncated from the N-terminal (F24 alpha-S1-casein) were used in combination with recombinantly-produced ovine (sheep) kappa casein (lacking PTMs), to form hybrid species casein micelles. As a control for native system casein micelles, bovine alpha-casein purified from cow's milk (Sigma Aldrich), and recombinantly-produced full length were used in combination with bovine kappa casein purified from cow's milk (Sigma Aldrich). 10.4 mg/ml of alpha-casein was mixed with 3.6 mg/ml kappa casein, and micelles were induced using 12.4 mM phosphate, 6.15 mM citrate, and 18.5 mM calcium. Micelles containing recombinantly produced proteins were induced using 20 mM phosphate, 10 mM citrate and 27 mM calcium. The resulting colloids were evaluated using dynamic light scattering (DLS) for particle size measurement and absorbance (A450) for turbidity measurement.

For particle sizing measurement, samples were diluted to a 1.4 mg/mL concentration of protein or less in filtered (220 nm) milliQ water. 50 ul samples were used for measurement, and three replicates were measured at a 173° detection angle over the amount of Zetasizer's small peak analysis mode. For turbidity measurements, samples were diluted to a concentration of 0.7 mg/ml in filtered (220 nm) milliQ water, and absorbance was measured at 450 nm in 1 ml cuvettes using Spectramax.

The resulting colloids were subjected to curd and cheesemaking, and the efficiency of cheesemaking was estimated (curd formation quality, stretch and melt quality, and yield). Samples were acidified by titrating 6.6% citric acid until the pH was between 5 and Samples were then renneted at room temperature using 0.15% rennet solution at 1.36% of the final micellar colloid volume. Curds were spun at 1,000×g for 1 minute, drained from liquid and submerged in a 70° C. water bath (duration dependent on curd size), stretched into mozzarella balls, placed on a wipe to remove excess moisture, and weighed.

FIG. 8 shows that native bovine alpha-casein, recombinant bovine alpha-S1-casein and recombinant truncated alpha-S1-casein were each able to form micelles with native bovine kappa casein. Most particle sizes of the main micellar population were in the range of 180-430 nm. The particle size of micelles containing recombinant truncated alpha-S1-casein and native kappa casein were slightly smaller (180-290 nm) compared to micelles having full-length recombinant alpha-S1-casein and kappa casein (270-430 nm). FIG. 7 also shows that recombinant bovine alpha-S1-casein and recombinant sheep kappa casein, both lacking PTMs, formed hybrid micelles in the size range of 190-320 nm. Recombinant truncated bovine alpha-S1-casein and sheep kappa casein colloid mostly gave larger particles (aggregates)>1000 nm, hybrid micelles in the size range 200-400 nm and smaller proportion of sub-micelles in the range of 70-90 nm. Table 12 shows the turbidity data.

TABLE 12
Average A450 of colloids subjected to particle size
analysis in FIG. 8 sheep

| | Native Bovine | Recombinant sheep |
|---|---|---|
| A450 | kappa casein | kappa casein |
| Native Bovine Alpha-S1-cassein | 4.28 | N/A |
| Recombinant Bovine | 1.98 | 2.75 |
| Alpha-S1-casein | | |
| Recombinant Truncated | 1.57 | 3.08 |
| Bovine Alpha-S1-casein | | |

The micellar colloid sample containing truncated bovine alpha-S1-casein with either native bovine kappa casein or recombinant sheep kappa casein produced very loose and weak curd, which was moist and had yogurt-like consistency. All other micellar colloid samples formed stable curds. All curds containing bovine alpha-casein with native kappa casein or hybrid sheep kappa casein made cheeses that stretched and melted well with similar yields. However, moist curd made from recombinant truncated alpha-S1-casein with native bovine kappa casein or with sheep kappa casein was not prone to melt when attempted to be shaped into a pasta-filata-like cheese. This non-native micellar colloid is not suitable for pasta-filata cheese making, but it is instead suitable for making soft and spreadable cheeses and in particular yogurts.

### Example 11: Hybrid Micelles from Bovine a Casein Variants and Truncated κ Casein, their Colloid Properties, and Cheesemaking

Native bovine alpha-casein purified from cow's milk (Sigma Aldrich) and recombinantly-produced bovine (cow) alpha-S1-casein (lacking PTMs) were used in combination with recombinantly-produced bovine kappa casein (lacking PTMs) lacking 16 amino acids from the C-terminus (rec_boyK-154I), to form hybrid casein micelles. As a control for native system casein micelles, bovine alpha-casein purified from cow's milk (Sigma Aldrich), and recombinantly-produced (cow) alpha-S1-casein (lacking PTMs) were used in combination with full-length bovine kappa casein purified from cow's milk (Sigma Aldrich) and recombinantly made bovine kappa casein (lacking PTMs). 10.4 mg/ml of alpha-casein was mixed with 3.6 mg/ml kappa casein, and micelles were induced using 12.4 mM phosphate, 6.15 mM citrate, and 18.5 mM calcium. Micelles containing recombinantly-produced proteins were induced using 20 mM phosphate, 10 mM citrate and 27 mM calcium. The resulting colloids were evaluated using dynamic light scattering (DLS) for particle size measurement and absorbance (A450) for turbidity measurement.

For particle sizing measurement, samples were diluted to a 1.4 mg/mL concentration of protein or less in filtered (220 nm) milliQ water. 50 ul samples were used for measurement, and three replicates were measured at a 173° detection angle over the amount of Zetasizer's small peak analysis mode. For turbidity measurements, samples were diluted to a concentration of 0.7 mg/ml in filtered (220 nm) milliQ water, and absorbance was measured at 450 nm in 1 ml cuvettes using Spectramax.

The resulting colloids were subjected to curd and cheesemaking, and the efficiency of cheesemaking was estimated (curd formation quality, stretch and melt quality, and yield). Samples were acidified by titrating 6.6% citric acid until the pH was between 5 and 5.8. Samples were then renneted at room temperature using 0.15% rennet solution at 1.36% of the final micellar colloid volume. Curds were spun at 1,000×g for 1 minute, drained from liquid and submerged in a 70° C. water bath (duration dependent on curd size), stretched into mozzarella balls, placed on a wipe to remove excess moisture, and weighed.

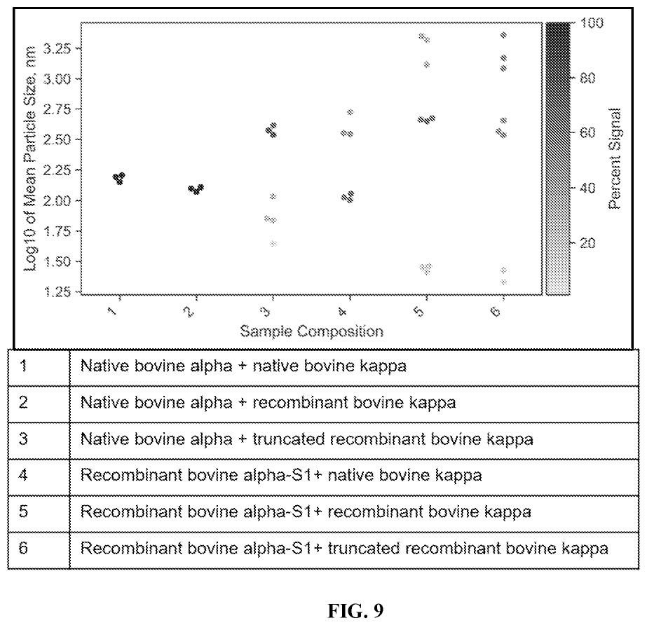

FIG. 9 shows that truncated bovine kappa casein is able to form non-native casein micelles with bovine alpha-casein, native or recombinant. Native bovine alpha-casein and recombinant truncated bovine kappa casein formed micellar colloid whose main particles population was in 300-530 nm range. Recombinant bovine alpha-S1-casein, lacking PTMs, and recombinant truncated bovine kappa casein formed micellar colloid whose main particles populations were aggregates >1000 nm and micelles in size range 340-610 nm (similar range as for native alpha-casein), alongside some sub-micelle population in 10-40 nm range.

TABLE 13
Average A450 of colloids subjected to particle size analysis in FIG. 9
kappa

| | Native kappa | Recombinant | Truncated kappa |
|---|---|---|---|
| | casein | kappa casein | casein |
| Native alpha | 4.21 | N/A | 13 |
| casein | | | |
| Recombinant | 0.67 | 1.52 | 2.69 |
| alpha casein | | | |

The non-native casein micelles containing truncated kappa casein combined with alpha-casein have a higher isoelectric point compared to micelles containing full-length kappa casein. Therefore, they had to be set at higher pH (˜5.8) during the acidification step to prevent protein precipitation. Non-native micelles and colloid from truncated kappa casein combined with native alpha-casein formed very loose curd and produced cheese that melted very well but did not stretch, and upon cooling, turned crumbly. Non-native micelles and colloid from truncated kappa casein combined with recombinant alpha-S1-casein made medium dense curd and produced again melty cheese, which did not stretch well and turned crumbly upon cooling. The truncated kappa casein non-native micelles are suitable for making crumbly cheeses, such as feta, and other dairy-like products that benefit from similar properties.

What is claimed is:

1. A hybrid micelle composition, comprising at least two casein proteins, wherein the two casein proteins are from different mammalian species; wherein at least one of the casein proteins is a recombinant protein; and wherein the casein proteins are associated in micellar form.

2. The hybrid micelle composition of claim 1, wherein the at least two casein proteins comprise a first alpha casein protein and a first kappa casein protein, wherein the first alpha casein protein and the first kappa casein protein are from different mammalian species.

3. The hybrid micelle composition of claim 2, wherein both of the first alpha casein protein and the first kappa casein protein are recombinant proteins.

4. A hybrid micelle composition, comprising two or more recombinant casein proteins, wherein at least two recombinant casein proteins are from different mammalian species; wherein the composition comprises a first alpha casein protein and a first kappa casein protein, and wherein the first alpha casein protein and the first kappa casein protein are associated in micellar form.

5. The hybrid micelle composition of claim 4, wherein the first alpha casein protein and the first kappa casein protein are from different mammalian species.

6. The hybrid micelle of any one of claims 2-5, comprising a second alpha casein protein.

7. The hybrid micelle composition of claim 6, wherein the first alpha casein protein and the second alpha casein protein are from different mammalian species.

8. The hybrid micelle composition of claim 6, wherein the first alpha casein protein and the second alpha casein protein are from the same mammalian species.

9. The hybrid micelle composition of any one of claims 6-8, wherein the second alpha casein protein is an alpha-S1-casein protein.

10. The hybrid micelle composition of any one of claims 6-8, wherein the second alpha casein protein is an alpha-S2-casein protein.

11. The hybrid micelle composition of any one of claims 6-10, wherein the second alpha casein is a truncated form of an alpha casein.

12. The hybrid micelle composition of claim 11, wherein the second alpha casein comprises a N-terminal truncation of alpha-S1-casein protein.

13. The hybrid micelle composition of claim 12, wherein the second alpha casein comprises lacks 1 to 59 N-terminal amino acids of alpha-S1-casein protein.

14. The hybrid micelle composition of claim 13, where the second alpha-S1-casein protein comprises a bovine amino acid sequence starting at amino acid 23, 24, 25, 26 or 60 of SEQ ID NO. 2.

15. The hybrid micelle composition of any one of claims 11-14, further comprising a third alpha casein, wherein the third alpha casein comprises a N-terminal truncation of alpha-S1-casein protein.

16. The hybrid micelle composition of claim 4, wherein the first alpha casein protein and the first kappa casein protein are from different mammalian species.

17. The hybrid micelle composition of any one of claims 2-16, comprising a second kappa casein protein.

18. The hybrid micelle composition of claim 17, wherein the first kappa casein protein and the second kappa casein protein are from different mammalian species.

19. The hybrid micelle composition of claim 17, wherein the first kappa casein protein and the second kappa casein protein are from the same mammalian species.

20. The hybrid micelle composition of any one of claims 2-19, wherein the first alpha casein protein is an alpha-S1-casein protein.

21. The hybrid micelle composition of any one of claims 2-19, wherein the first alpha casein protein is an alpha-S2-casein protein.

22. The hybrid micelle composition according to any of claims 2-21, wherein the first alpha casein protein is a bovine alpha casein protein.

23. The hybrid micelle composition according to any of claims 2-19, wherein the first alpha casein protein comprises an amino acid sequence of SEQ ID NO. 1-11, 18-20, 30-32 or 39-41, or an amino acid sequence with at least 90% sequence identity to any one of SEQ ID NO. 1-11, 18-20, 30-32 or 39-41.

24. The hybrid micelle composition of claim 22 or claim 23, wherein the first kappa casein protein is a kappa casein protein from any one of: ovine, caprine, equine, and camel kappa casein proteins.

25. The hybrid micelle composition of claim 21 or claim 22, wherein the first kappa casein protein comprises an amino acid sequence of any one of SEQ ID NOs. 53-70, or an amino acid sequence with at least 90% sequence identity to any one of SEQ ID NOs. 53-70.

26. The hybrid micelle composition according to any of claims 2-20, wherein the first alpha casein protein is an ovine alpha casein protein.

27. The hybrid micelle composition according to any of claims 2-18, wherein the first alpha casein protein comprises an amino acid sequence of any one of SEQ ID NOs. 12-14 or 33-35 or an amino acid sequence with 90% sequence identity to any one of SEQ ID NOs. 12-14 or 33-35.

28. The hybrid micelle composition of claim 25 or claim 26, wherein the first kappa casein protein is a kappa casein protein selected from the group consisting of bovine, caprine, equine, and camel.

29. The hybrid micelle composition of claim 25 or claim 26, wherein the first kappa casein protein comprises an amino acid sequence according to any one of SEQ ID NOs. 48-52 or 56-70, or an amino acid sequence with at least 90% sequence identity to any one of SEQ ID NOs. 48-52 or 56-70.

30. The hybrid micelle composition according to any of claims 2-18, wherein the first alpha casein protein is a caprine alpha casein protein.

31. The hybrid micelle composition according to any of claims 2-18, wherein the first alpha casein protein comprises an amino acid sequence of any one of SEQ ID NOs. 15-17 or 36-38, or an amino acid sequence with at least 90% sequence identity to any one of SEQ ID NOs. 15-17 or 36-38.

32. The hybrid micelle composition of claim 29 or claim 30, wherein the first kappa casein protein is a kappa casein protein selected from the group consisting of ovine, bovine, equine and camel.

33. The hybrid micelle composition of claim 29 or claim 30, wherein the first kappa casein protein comprises an amino acid sequence of any one of SEQ ID NOs. 48-55 or 59-70 or an amino acid sequence with at least 90% sequence identity to any one of SEQ ID NOs. 48-55 or 59-70.

34. The hybrid micelle composition of any one of claims 2-21, wherein the first alpha casein is a truncated form of an alpha casein.

35. The hybrid micelle composition of claim 33, wherein the first alpha casein comprises a N-terminal truncation of alpha-S1-casein protein.

36. The hybrid micelle composition of claim 34, wherein the first alpha casein lacks 1 to 59 N-terminal amino acids of alpha-S1-casein protein.

37. The hybrid micelle composition of claim 35, where the first alpha-S1-casein protein comprises a bovine amino acid sequence starting at amino acid 23, 24, 25 or 26 of SEQ ID NO. 2.

38. The hybrid micelle composition according to any of claims 1-36, wherein the micellar form does not include a beta casein protein or a derivative thereof.

39. The hybrid micelle composition according to any of claims 1-36, wherein the hybrid micelle comprises a beta casein protein.

40. The hybrid micelle composition of claim 38, wherein the beta casein protein is from the same species as the first alpha casein protein of the hybrid micelle composition.

41. The hybrid micelle composition of claim 38, wherein the beta casein protein is from the same species as the first kappa casein protein of the hybrid micelle composition.

42. The hybrid micelle composition of claim 38, wherein the beta casein protein is from a species that is different from the first alpha casein protein and the first kappa casein protein of the hybrid micelle composition.

43. The hybrid micelle composition of claim 38, wherein the beta casein protein is selected from the group consisting of: a full-length beta casein protein, a gamma casein protein, and an alternate truncation of beta casein protein.

44. The hybrid micelle composition of any one of claims 38-42, wherein the beta casein protein is a recombinant protein.

45. The hybrid micelle composition according to any of claims 2-43 wherein the first alpha casein protein is not phosphorylated or is substantially reduced in phosphorylation as compared to native alpha casein protein.

46. The hybrid micelle composition according to any of claims 2-43, wherein the first alpha casein protein comprises a phosphorylation pattern that differs from native alpha casein protein.

47. The hybrid micelle composition according to any of claims 2-45, wherein the first kappa casein protein is not glycosylated or is substantially reduced in glycosylation as compared to native kappa casein protein.

48. The hybrid micelle composition according to any of claims 2-45, wherein the first kappa casein protein comprises a glycosylation pattern that differs from native kappa casein protein.

49. The hybrid micelle composition according to any of claims 2-47, wherein the ratio of total alpha casein protein to total kappa casein protein in the composition is about 1:1 to about or about 3:1 to about 5:1.

50. The hybrid micelle composition of any one of claims 2-49, wherein the first alpha casein protein, the first kappa casein protein or both the first alpha casein protein and the first kappa casein protein are produced from a plant or a mammalian host cell.

51. The hybrid micelle composition of any one of claims 2-49, wherein the first alpha casein protein, the first kappa casein protein or both the first alpha casein protein and the first kappa casein protein are produced from a microbial host cell.

52. The hybrid micelle composition of claim 51, wherein the microbial host cell is selected from the group consisting of a bacteria, a yeast, and a fungus.

53. The hybrid micelle composition of claim 52, wherein the microbial host cell is a bacteria selected from the group consisting of Lactococci sp., Lactococcus lactis, Bacillus subtilis, Bacillus amyloliquefaciens, Bacillus licheniformis, Bacillus megaterium, Brevibacillus choshinensis, Mycobacterium smegmatis, Rhodococcus erythropolis and Corynebacterium glutamicum, Lactobacilli sp., Lactobacillus fermentum, Lactobacillus casei, Lactobacillus acidophilus, Lactobacillus plantarum, Synechocystis sp. 6803, and E. coli.

54. A protein powder comprising the hybrid micelle composition of any one of claims 1-53.

55. A colloid comprising the hybrid micelle composition according to any of claims 1-53.

56. A dairy-like product derived from the hybrid micelle composition according to any of claims 1-53.

57. The dairy-like product of claim 56, wherein the hybrid micelle composition provides one or more dairy-like features selected from the group consisting of texture, melt, stretch, turbidity, and appearance.

58. The dairy-like product of claim 56, wherein the dairy-like product is selected from the group consisting of milk, cream, ice cream and yogurt.

59. The dairy-like product of claim 56, wherein the dairy-like product is a cheese analogue.

60. The dairy-like product of claim 56, wherein the dairy-like product is a coagulated colloid of the hybrid micelle composition.

61. The dairy-like product of claim 60, wherein the coagulated colloid is cheese curd.

62. The dairy-like product of claim 60, wherein the coagulated colloid is cheese.

63. The dairy-like product of claim 62, wherein the cheese is a soft cheese, a hard cheese or an aged cheese.

64. The cheese composition of claim 62, wherein the cheese is selected from the group consisting of pasta-filata like cheese, paneer, cream cheese, and cottage cheese.

65. The cheese composition of claim 62, wherein the cheese is selected from the group consisting of mozzarella, cheddar, swiss, brie, camembert, feta, halloumi, gouda, edam, cheddar, manchego, swiss, Colby, muenster, blue cheese and parmesan.

66. The dairy-like product of claim 60, wherein the coagulated colloid is yogurt.

67. The dairy-like product according to any of claims 56-66, wherein the dairy-like product does not include any animal-sourced dairy protein.

68. The dairy-like product according to any of claims 56-66, wherein the dairy-like product does not include any dairy-related protein other than caseins.

69. The dairy-like product according to any of claims 56-66, further comprising at least one additional protein other than caseins.

70. The dairy-like product of claim 69, wherein the at least one additional protein is a dairy related protein other than caseins.

71. The dairy-like product of claim 70, wherein the dairy-related protein is a whey protein.

72. A method for producing a hybrid micelle composition, comprising: providing an alpha casein protein and a kappa casein protein, wherein at least one of the alpha casein protein and the kappa casein protein is a recombinant protein, and wherein (a) the alpha casein protein and the kappa casein protein are from a different mammalian species, and/or (b) the alpha casein protein comprises a deletion in the amino acid sequence as compared to a native alpha casein protein sequence; and combining the alpha casein protein and a kappa casein protein and at least one salt under conditions wherein alpha casein protein and a kappa casein protein form a micellar form in a liquid colloid.

73. The method of claim 72, wherein the salt is selected from the group consisting of a calcium salt, a citrate salt, a phosphate salt, and any combination thereof.

74. The method of claim 72 or claim 73, wherein the micellar form further comprises a beta casein protein.

75. The method of claim 72 or claim 73, wherein the micellar form lacks a beta casein protein.

76. The method according to any of claims 72-74, wherein the beta casein protein comprises a full-length beta casein protein, a gamma casein protein or an alternate truncation of beta casein protein.

77. The method according to any of claims 72-76, further comprising subjecting the liquid colloid to a first condition to form coagulates.

78. The method of claim 77, wherein the first condition is the addition of acid or acidification of the liquid colloid with a microorganism.

79. The method of claim 77 or claim 78, wherein the method further comprises subjecting the coagulates to a hot water treatment and optionally stretching, to form a filata-type cheese.

80. The method of claim 76, wherein the method further comprises subjecting the coagulates to a renneting agent to form a rennetted curd.

81. The method of claim 80, wherein the renneting agent is a microbially-derived chymosin enzyme.

82. The method of claim 80 or claim 81, wherein the method further comprises aging and maturing the rennetted curd to form a cheese-like food product.

83. The method of claim 80 or claim 81, wherein the method further comprises subjecting the rennetted curd to a hot water treatment and optionally stretching, to form a filata-type cheese food product.

84. The method according to any one of claims 72-83, wherein the recombinantly produced alpha casein protein and/or kappa casein protein are produced from a plant or a mammalian host cell.

85. The method according to any of claims 72-83, wherein the recombinantly produced alpha casein protein and/or kappa casein protein are produced from a microbial host cell.

86. The method of claim 85, wherein the microbial host cell is selected from the group consisting of a bacteria, a yeast, and a fungus.

87. The method of claim 85, wherein the microbial host cell is a bacteria selected from the group consisting of Lactococci sp., Lactococcus lactis, Bacillus subtilis, Bacillus amyloliquefaciens, Bacillus licheniformis, Bacillus megaterium, Brevibacillus choshinensis, Mycobacterium smegmatis, Rhodococcus erythropolis and Corynebacterium glutamicum, Lactobacilli sp., Lactobacillus fermentum, Lactobacillus casei, Lactobacillus acidophilus, Lactobacillus plantarum, Synechocystis sp. 6803, and E. coli.

88. The method according to any of claims 72-87, further comprising drying the liquid colloid to produce a micellar casein containing protein powder.

89. The method of claim 88, wherein the drying comprises spray-drying, freeze-drying or drum drying.

90. The method according to any of claims 72-89, further comprising subjecting the liquid colloid to salt or acid precipitation to produce a caseinate-like protein powder.

91. A protein powder produced by the method according to any of claim 72-77 or 83-90.

92. The protein powder of claim 91, wherein the powder is a spray-dried, freeze-dried or drum dried powder.

93. A dairy-like product comprising the protein powder of claim 91 or claim 92.

94. The dairy-like product of claim 93, wherein the dairy-like product is selected from the group consisting of milk, cream, ice-cream, yogurt, mozzarella cheese analogue, curd and cheese.

95. A hybrid micelle composition, comprising an alpha casein protein and a recombinant truncated kappa casein protein, wherein the alpha casein protein and the recombinant truncated kappa casein protein are associated in micellar form.

96. The hybrid micelle of claim 95, wherein the recombinant truncated kappa casein protein comprises a C-terminal truncation of a kappa casein protein amino acid sequence.

97. The hybrid micelle composition of claim 95 and claim 96, wherein the kappa casein protein is from a species selected from the group consisting of bovine, ovine and caprine.

98. The hybrid micelle of claim 97, wherein the kappa casein protein comprises a bovine amino acid sequence truncated after amino acid 153 of SEQ ID NO. 49.

99. The hybrid micelle composition of any one of claims 95-98, wherein the alpha casein protein is a recombinant protein.

100. The hybrid micelle composition of any one of claims 95-99, wherein the alpha casein protein lacks one or more native post-translational modifications.

101. The hybrid micelle composition of any one of claims 95-98, wherein the alpha casein protein is a native protein.

102. The hybrid micelle composition of any one of claims 95-101, further comprising aggregates of the alpha casein protein and the recombinant truncated kappa casein protein.

103. The hybrid micelle composition of claim 102, wherein the aggregates are about or greater than 1000 nm in size.

104. The hybrid micelle composition of any one of claims 95-103, wherein the micelles are in a size range of 300-610 nm, 300-530 nm, or 340-610 nm.

105. A dairy-like product comprising the hybrid micelle composition of any one of claims 95-104.

106. The dairy-like product of claim 105, wherein the dairy product is a curd.

107. The dairy-like product of claim 105, wherein the dairy product is a cheese.

108. The dairy-like product of claim 107, wherein the cheese has a texture, mouthfeel, crumble, melt or any combination thereof desirable for a crumbly cheese, such as feta, asiago, blue, cheddar, cheshire, cotija, paneer.

109. A pasta-filata cheese product comprising: between 70-85% of an alpha casein; between of an ovine kappa casein; wherein at least one of the alpha casein and the kappa casein is a recombinant protein and wherein the alpha casein and the kappa casein are associated in micellar form; a fat in range 0-50%; and one or more salts from group of: calcium, phosphate and citrate; wherein the cheese product is extremely melty and stretchy and displays melt and stretch superior to cheese derived using bovine kappa casein, and wherein the cheese product has pasta-filata like structure and/or firmness.

110. A pasta-filata cheese product comprising: between 70-85% of an alpha casein; between 15-30% of a bovine or caprine kappa casein; wherein at least one of the alpha casein and the kappa casein is a recombinant protein and wherein the alpha casein and the kappa casein are associated in micellar form; a fat in range 0-50%; and one or more salts from group of: calcium, phosphate and citrate; wherein the cheese product has a pasta-filata-like melt, stretch, structure and/or firmness.

111. The pasta-filata cheese product of any one of claims 109-110, wherein the pasta-filata cheese product does not comprise any dairy-obtained components.

112. The pasta-filata cheese product of any one of claims 109-110, wherein the at least one of the alpha casein and kappa casein is an altered form of the protein.

113. A hard-matured cheese product comprising: between 70-85% of an alpha casein; between 15-30% of an ovine kappa casein; wherein the alpha casein and the kappa casein are associated in a combination of micellar and aggregate forms; a fat in range 0-50%; and one or more salts from group of: calcium, phosphate and citrate; wherein the cheese product has a swiss-like or cheddar-like consistency and is capable of melting.

114. The hard-matured cheese product of claim 113, wherein the hard-matured cheese product does not comprise any dairy-obtained components.

115. The hard-matured cheese product of claim 113, wherein the at least one of the alpha casein and kappa casein is an altered form of the protein.

116. A very hard dry cheese product comprising: between 70-85% of an alpha casein; between 15-30% of an ovine kappa casein; wherein the alpha casein and the kappa casein are associated in a combination of micellar and aggregate forms; a fat in range 0-50%; and one or more salts from group of: calcium, phosphate and citrate; wherein the cheese product has a parmesan-like crumbly consistency and is capable of melting.

117. The very hard dry cheese product of claim 116, wherein the very hard dry cheese product does not comprise any dairy-obtained components.

118. The very hard dry cheese product of claim 113, wherein the at least one of the alpha casein and kappa casein is an altered form of the protein.

119. A goat-like soft and spreadable cheese product comprising: between 70-85% of an alpha casein lacking PTMs; between 15-30% of a caprine kappa casein; wherein at least one of the alpha casein and the kappa casein is a recombinant protein and wherein the alpha casein and the kappa casein are associated in micellar form; a fat in range 0-50%; and one or more salts from group of: calcium, phosphate and citrate; wherein the cheese product has a soft and spreadable consistency.

120. The goat-like soft and spreadable cheese product of claim 119, wherein the goat-like soft and spreadable cheese product does not comprise any dairy-obtained components.

121. The goat-like soft and spreadable cheese product of claim 119, wherein the at least one of the alpha casein and kappa casein is an altered form of the protein.

122. A soft cheese product comprising: between 70-75% of a truncated alpha casein; between 25-30% of a kappa casein; wherein at least one of the alpha casein and the kappa casein is a recombinant protein and wherein the alpha casein and the kappa casein are associated in micellar form; a fat in range 0-50%; and one or more salts from group of: calcium, phosphate and citrate; wherein the cheese product has a cottage-cheese like consistency and moisture.

123. The soft cheese product of claim 122, wherein the soft cheese product does not comprise any dairy-obtained components.

124. The soft cheese product of claim 122, wherein the at least one of the alpha casein and kappa casein is an altered form of the protein.

125. A yogurt-like product comprising: between 70-75% of a truncated alpha casein and between 25-30% of a kappa casein; wherein at least one of the alpha casein and the kappa casein is a recombinant protein and wherein the alpha casein and the kappa casein are associated in micellar form; a fat and a sugar, and one or more salts from group of: calcium, phosphate, citrate; wherein the yogurt-like product has a moist and loose consistency.

126. The yogurt-like product of claim 125, wherein the yogurt-like product does not comprise any dairy-obtained components.

127. The yogurt-like product of claim 125, wherein the at least one of the alpha casein and kappa casein is an altered form of the protein.

128. A crumbly cheese product comprising: between 70-75% of an alpha casein; between a 25-30% of a truncated bovine kappa casein; wherein the alpha casein and the kappa casein are associated in a combination of micellar and aggregate forms; a fat in range 0-50%; and one or more salts from group of: calcium, phosphate and citrate; wherein the cheese product has a crumbly consistency and is capable of melting.

129. The crumbly cheese product of claim 128, wherein the crumbly cheese product does not comprise any dairy-obtained components.

130. The crumbly cheese product of claim 128, wherein the at least one of the alpha casein and kappa casein is an altered form of the protein.

131. A method for regulating the yield of curd comprising:
providing an alpha casein protein and a kappa casein protein;
combining the alpha casein protein and a kappa casein protein and at least one salt under conditions wherein alpha casein protein and a kappa casein protein form a micellar form in a liquid colloid; and
coagulating the liquid colloid to form a curd,
wherein the ratio of alpha casein protein to a kappa casein protein regulates the yield of curd.

132. The method of claim 131, wherein the alpha casein protein and the kappa casein protein are from different species.
